(19) United States
(12) Patent Application Publication
(10) Pub. No.: US 2026/0184807 A1
(43) Pub. Date: Jul. 2, 2026

(54) Methods for Treating Rheumatoid Arthritis Using a Syndecan-1 Inhibitor or Syntenin-1 Inhibitor

(71) Applicants: The United States Government as represented by the Department of Veterans Affairs, Washington, DC (US); The Board of Trustees of the University of Ilinois, Urbana, IL (US)

(72) Inventors: Shiva Shahrara, Chicago, IL (US); Anja Meyer, Chicago, IL (US)

(21) Appl. No.: 19/130,189

(22) PCT Filed: Nov. 17, 2023
(86) PCT No.: PCT/US2023/080337

§ 371 Date: May 15, 2025

Related U.S. Application Data
(60) Provisional application No. 63/426,631, filed on Nov. 18, 2022.
(60) Provisional application No. 63/481,277, filed on Jan. 24, 2023.
(60) Provisional application No. 63/505,922, filed on Jun. 2, 2023.

(51) Int. Cl. C07K 16/28; A61K 38/08; A61K 38/17; A61K 40/11; A61K 40/15; A61K 40/31; A61K 40/41; A61K 47/68; A61P 19/02

(52) U.S. Cl. CPC C07K 16/2896; A61K 38/08; A61K 38/177; A61K 40/11; A61K 40/15; A61K 40/31; A61K 40/41; A61K 47/68033; A61K 47/6849; A61P 19/02; C07K 16/2851

(57) ABSTRACT

Disclosed herein are methods of treating or preventing rheumatoid arthritis by administering a syndecan-1 inhibitor or a synthenin-1 inhibitor capable of preventing syndecan-1 from binding to the PDZ-2 domain of syntenin-1. Also disclosed herein are methods of reducing or ameliorating one or more symptoms of rheumatoid arthritis, reducing syntenin-1 or syndecan-1 in synovial fluid or blood, reducing synovial inflammation, reducing cartilage degradation, reducing one or more inflammatory interferon transcription factors, reducing one or more monokines, reducing expression of one or more glycolytic factors, or increasing expression of one or more oxidative intermediates or enzymes in a subject.

### CROSS REFERENCE TO RELATED APPLICATIONS

This application claims the benefit of the filing date of U.S. Provisional Application No. 63/426,631, filed on Nov. 18, 2022, U.S. Provisional Application 63/481,277, filed on Jan. 24, 2023, and U.S. Provisional Application 63/505,922, filed on Jun. 2, 2023. The content of these earlier filed applications is hereby incorporated by reference in its entirety.

### STATEMENT REGARDING FEDERALLY FUNDED RESEARCH

This invention was made with government support under grant number and BX002286 awarded by United States Department of Veterans Affairs, and grant numbers AI167155 and AI147697 awarded by the National Institutes of Health. The government has certain rights in the invention.

### REFERENCE TO A SEQUENCE LISTING

The present application contains a Sequence Listing that is submitted concurrent with the filing of this application in XML format, containing the file name “37759_0502P1_SL.xml,” created on Nov. 2, 2023, and having a size of 4,096 bytes. The Sequence Listing is hereby incorporated by reference pursuant into the present application in its entirety.

### BACKGROUND

Rheumatoid arthritis is a chronic disorder with no cure. Current treatment options for rheumatoid arthritis include anti-inflammatory agents such as oral non-steroidal anti-inflammatory drugs (NSAIDs), corticosteroids, and disease modifying anti-rheumatic drugs (DMARDs). NSAIDS and corticosteroids are short-acting, while DMARDs can take months to provide a clinical effect. These options are limited to ameliorating pain and improving function temporarily without impacting disease progression. Moreover, existing anti-inflammatory agents such as the oral NSAIDs increase risk of gastric ulceration and cardiovascular events, and can cause renal and hepatic toxicity. Steroid medications are associated with accelerated osteoporosis and in some cases lead to steroid toxicity. These side effects and toxicities limit their use in many patients with rheumatoid arthritis. Thus, a need exists for the treatment, prevention, and management of rheumatoid arthritis.

### SUMMARY OF THE INVENTION

Disclosed herein are methods of treating or preventing rheumatoid arthritis in a subject, the methods comprising administering to the subject a therapeutically effective amount of a syndecan-1 inhibitor.

Disclosed herein are methods of reducing syntenin-1 in synovial fluid or blood in a subject, the methods comprising administering to the subject a therapeutically effective amount of a syndecan-1 inhibitor.

Disclosed herein are methods of reducing chemokine (C—C motif) ligand 2 (CCL2) levels in a subject, the methods comprising administering to the subject a therapeutically effective amount of a syndecan-1 inhibitor.

Disclosed herein are methods of reducing cartilage degradation in a subject, the methods comprising administering to the subject a therapeutically effective amount of a syndecan-1 inhibitor.

Disclosed herein are methods of reducing synovial inflammation in a subject, the methods comprising administering to the subject a therapeutically effective amount of a syndecan-1 inhibitor.

Disclosed herein are methods of reducing or ameliorating one or more symptoms of rheumatoid arthritis in a subject, the methods comprising administering to the subject a therapeutically effective amount of a syndecan-1 inhibitor.

Disclosed herein are methods of reducing one or more inflammatory interferon transcription factors in a subject, the methods comprising administering to the subject a therapeutically effective amount of a syndecan-1 inhibitor.

Disclosed herein are methods of reducing one or more monokines in subject, the methods comprising administering to the subject a therapeutically effective amount of a syndecan-1 inhibitor.

Disclosed herein are methods of reducing expression of one or more of glycolytic factors in subject, the methods comprising administering to the subject a therapeutically effective amount of a syndecan-1 inhibitor.

Disclosed herein are methods of increasing expression of one or more of oxidative intermediates or enzymes in subject, the methods comprising administering to the subject a therapeutically effective amount of a syndecan-1 inhibitor.

Disclosed herein are methods of treating or preventing rheumatoid arthritis in a subject, the methods comprising administering to the subject a therapeutically effective amount of a syntenin-1 inhibitor.

Disclosed herein are methods of reducing syntenin-1 in synovial fluid or blood in a subject, the methods comprising administering to the subject a therapeutically effective amount of a syntenin-1 inhibitor.

Disclosed herein are methods of reducing chemokine (C—C motif) ligand 2 (CCL2) levels in a subject, the methods comprising administering to the subject a therapeutically effective amount of a syntenin-1 inhibitor.

Disclosed herein are methods of reducing cartilage degradation in a subject, the methods comprising administering to the subject a therapeutically effective amount of a syntenin-1 inhibitor.

Disclosed herein are methods of reducing synovial inflammation in a subject, the methods comprising administering to the subject, the method comprising administering to the subject a therapeutically effective amount of a syntenin-1 inhibitor.

Disclosed herein are methods of reducing or ameliorating one or more symptoms of rheumatoid arthritis in a subject, the methods comprising administering to the subject a therapeutically effective amount of a syntenin-1 inhibitor.

Disclosed herein are methods of reducing one or more inflammatory interferon transcription factors in subject, the methods comprising administering to the subject a therapeutically effective amount of a syntenin-1 inhibitor.

Disclosed herein are methods of reducing one or more monokines in subject, the methods comprising administering to the subject a therapeutically effective amount of a syntenin-1 inhibitor.

Disclosed herein are methods of reducing expression of one or more of glycolytic factors in subject, the method comprising administering to the subject a therapeutically effective amount of a syntenin-1 inhibitor.

Disclosed herein are methods of increasing expression of one or more of oxidative intermediates or enzymes in subject, the methods comprising administering to the subject a therapeutically effective amount of a syntenin-1 inhibitor.

Disclosed herein are method of treating or preventing juvenile idiopathic arthritis in a subject, the methods comprising administering to the subject a therapeutically effective amount of a syndecan-1 inhibitor.

Disclosed herein are methods of treating or preventing psoriatic arthritis in a subject, the methods comprising administering to the subject a therapeutically effective amount of a syndecan-1 inhibitor.

Disclosed herein are methods of treating or preventing ankylosing spondylitis in a subject, the methods comprising administering to the subject a therapeutically effective amount of a syndecan-1 inhibitor.

Disclosed herein are methods of treating or preventing Crohn's disease in a subject, the methods comprising administering to the subject a therapeutically effective amount of a syndecan-1 inhibitor.

Disclosed herein are methods treating or preventing ulcerative colitis in a subject, the methods comprising administering to the subject a therapeutically effective amount of a syndecan-1 inhibitor.

Disclosed herein are methods treating or preventing plaque psoriasis in a subject, the methods comprising administering to the subject a therapeutically effective amount of a syndecan-1 inhibitor.

Disclosed herein are methods treating or preventing hidradenitis suppurativa in a subject, the methods comprising administering to the subject a therapeutically effective amount of a syndecan-1 inhibitor.

Disclosed herein are methods treating or preventing uveitis in a subject, the methods comprising administering to the subject a therapeutically effective amount of a syndecan-1 inhibitor.

Disclosed herein are methods of reducing or ameliorating one or more symptoms of idiopathic juvenile arthritis in a subject, the methods comprising administering to the subject a therapeutically effective amount of a syndecan-1 inhibitor.

Disclosed herein are methods of reducing or ameliorating one or more symptoms of psoriatic arthritis in a subject, the method comprising administering to the subject a therapeutically effective amount of a syndecan-1 inhibitor.

Disclosed herein are methods of reducing or ameliorating one or more symptoms of ankylosing spondylitis in subject, the methods comprising administering to the subject a therapeutically effective amount of a syndecan-1 inhibitor.

Disclosed herein are methods of treating or preventing juvenile idiopathic arthritis in a subject, the methods comprising administering to the subject a therapeutically effective amount of a syntenin-1 inhibitor.

Disclosed herein are methods of treating or preventing psoriatic arthritis in a subject, the methods comprising administering to the subject a therapeutically effective amount of a syntenin-1 inhibitor.

Disclosed herein are methods of treating or preventing ankylosing spondylitis in a subject, the methods comprising administering to the subject a therapeutically effective amount of a syntenin-1 inhibitor.

Disclosed herein are methods of treating or preventing Crohn's disease in a subject, the methods comprising administering to the subject a therapeutically effective amount of a syntenin-1 inhibitor.

Disclosed herein are methods treating or preventing ulcerative colitis in a subject, the method comprising administering to the subject a therapeutically effective amount of a syntenin-1 inhibitor.

Disclosed herein are methods treating or preventing plaque psoriasis in a subject, the methods comprising administering to the subject a therapeutically effective amount of a syntenin-1 inhibitor.

Disclosed herein are methods treating or preventing hidradenitis suppurativa in a subject, the methods comprising administering to the subject a therapeutically effective amount of a syntenin-1 inhibitor.

Disclosed herein are methods treating or preventing uveitis in a subject, the methods comprising administering to the subject a therapeutically effective amount of a syntenin-1 inhibitor.

Disclosed herein are methods of reducing or ameliorating one or more symptoms of idiopathic juvenile arthritis in a subject, the methods comprising administering to the subject a therapeutically effective amount of a syntenin-1 inhibitor.

Disclosed herein are methods of reducing or ameliorating one or more symptoms of psoriatic arthritis in a subject, the methods comprising administering to the subject a therapeutically effective amount of a syntenin-1 inhibitor.

Disclosed herein are methods of reducing or ameliorating one or more symptoms of ankylosing spondylitis in subject, the methods comprising administering to the subject a therapeutically effective amount of a syntenin-1 inhibitor.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIGS. 1A-M show Syntenin-1 and syndecan-1 (SDC-1) expression is linked to rheumatoid arthritis (RA) clinical manifestation and is mutually enhanced by LPS/IFNγ stimulation in macrophages (MΦs). FIGS. 1A and 1B show that Syntenin-1 transcription (FIG. TA) and protein levels (FIG. 1B) were quantified in osteoarthritis (OA) (n=9) and RA (n=9-10) synovial fluids by qRT-PCR or ELISA. FIGS. 1C and 1D show that synovial tissues from normal (NL0 (n=6), osteoarthritis (OA) (n=7), or RA (n=7) individuals were used to determine Syntenin-1 presentation (FIG. 1C) and its relative expression was scored in the lining, sublining and blood vessels (BV) (on a 0-5 scale) (FIG. 1D). FIG. TE shows that RA synovial tissue (STs) were fluorescently stained to authenticate the colocalization of SDC-1 with Syntenin-1 and their expression on CD14+ cells in presence of DAPI. FIG. 1F and FIG. 1G show relative expression of Syntenin-1 (FIG. 1F) or SDC-1 (FIG. 1G) was determined by RNAseq (n=87) in RA synovial tissue biopsies and linked to the number of CD68+MΦs quantified by histology scoring (score 0-4). FIGS. 1H to IK show that CD14+CD16−myeloid cells FIG. 1H or SDC-1 transcript levels (FIG. 1I) were evaluated by RNAseq (n=90) and correlated with RA ultrasound-guided synovial tissue thickness (score 0-3). Blood Syntenin-1 (FIG. 1J, n=67) or synovial tissue SDC-1 (FIG. 1K, n=87) transcript level was quantified by RNAseg18 and linked to CCP or ESR. FIG. 1L shows that human myeloid cells were stimulated with IFNγ and LPS, TNFα. IL-1β and IL-6 (100 ng/mL), and expression of Syntenin-1 and SDC-1 was analyzed by Western blot. β-actin served as a loading control. FIG. 1M shows that RA MΦs were untreated or treated with LPS/IFNγ (100 ng/mL) with or without TNFαi, IL6R Ab, Jaki (Tofacitinib; 10 μg/mL) or SDC-1 antibody (SDCab, 1:100), and transcription levels of CCL5 were determined by qRT-PCR (n=4). Data are presented as mean±SEM; significant differences were determined by the Mann-Whitney test, 2way-ANOVA, or one-way ANOVA. For RNAseq data, spearman rank correlation was used. *p<0.05, **p<0.01, ***p<0.001, ****p<0.0001.

FIGS. 2A-K show ligation of Syntenin-1 to SDC-1 expands RA MΦ inflammatory profile independent of IL 5R or PDZ1 function. FIG. 2A shows that human myeloid cells were treated with Syntenin-1 (SYN1:1000 ng/mL) for 0-60 min and phosphorylation of Src, AKT, STAT1, STAT3, p38, ERK and JNK, and degradation of IxB was determined by western blot analysis and β-actin served as a loading control. FIGS. 2B-2E show that RA MΦs were treated with PBS (ctrl) or Syntenin-1 (1000 ng/mL) for 6 hour or 24 hours. Transcription of IRFs (FIG. 2B) and the inflammatory monokines (FIG. 2C) was assessed by qRT-PCR (n=3-10), and protein levels of IL-6 (FIG. 2D) or TNFα (FIG. 2E) were determined in the conditioned media by ELISA (n=10). FIGS. 2F-2K show that RA MΦs were treated with PBS or Syntenin-1 (1000 ng/mL) in the presence or absence of SDC-1 Ab (SDCab; 1:100), IL-5R Ab (IL5Ra; 2 μg/mL), or PDZli (PDZ1; 10 μM) for 6 hours or 24 hours before quantifying TNFα (FIG. 2F), CCL2 (FIG. 2G), TLRs (FIG. 2I), TGFβ (FIG. 2J), or IL-1β (FIG. 2K) mRNA levels by qRT-PCR or CCL2 protein levels (FIG. 2H) by ELISA (n=4-8). Data are presented as mean±SEM: significant differences were determined by the Mann-Whitney test, two-way-ANOVA, or one-way ANOVA. *p<0.05, **p<0.01, ***p<0.001, ****p<0.0001. ANOVA, analysis of variance.

FIGS. 3A-M show that RA MΦ metabolic reprogramming is potentiated by activation of the Syntenin-1/SDC-1 pathway. FIG. 3A show that RA MΦs were untreated (ctrl) or treated with Syntenin-1 (SYN1; 1000 ng/mL) for 6-48 hours before determining the expression of GLUT1, HK2, PFK2 and LDHA by Western blot analysis. β-actin served as a loading control. (B, C) RA MΦs were treated with PBS (ctrl) or Syntenin-1 (1000 ng/mL) for 6 hours and transcription levels of HIF1α (n=10) (FIG. 3B). GLUT1, RAPTOR, HK2, PFK2, PDK1, PKM2 (FIG. 3C) were quantified by qRT-PCR (n=6). FIG. 3D shows that RA monocyte-differentiated MΦs were treated with PBS or Syntenin-1 (1000 ng/mL) for 24 hours before measuring lactate protein levels colorimetrically (n=16). FIGS. 3E-3G show that RA monocyte-differentiated MΦs (2×10⁵ cells/well) were treated with PBS or Syntenin-1 (1000 ng/mL) and % glycolysis increase (FIGS. 3E, 3F) and % oxidative phosphorylation decrease (FIGS. 3E, 3G) were calculated by Seahorse XF Real-Time ATP Rate Assay Kit (n=7). FIGS. 3H-3M show that RA monocyte-differentiated MΦs were treated with PBS or syntenin-1 (1000 ng/mL) in the presence or absence of SDC-1 Ab (SDCab; 1:100) and/or IL-5R Ab (IL5Ra; 2 μg/mL), or PDZli (PDZ1; 10 μM) (FIGS. 3H and 3K-3L), and 2-DG (5 mM), mTORi (1 μM), or HIF1αi (2 μM) (FIG. 3I, 3J). FIGS. 3H, 3I) after 24 hours impact of treatment was determined on CD14+CD86+ GLUT1+ frequency by flow cytometry (n=4-5). FIGS. 3J-3M show that mRNA expression of CCL2 (FIG. 3J), PPARγ (FIG. 3K), and AMPK (FIG. 3L), or oxidative enzymes (FIG. 3M) was determined after 6 hours by qRT-PCR (n=4-6). Data are presented as mean±SEM; significant differences were determined by Mann-Whitney test, Student's t-test, two-way-ANOVA, or one-way ANOVA. *p<0.05, **p<0.01, ***p<0.001, ****p<0.0001. ANOVA, analysis of variance.

FIGS. 4A-L show that Syntenin-1 enhances Th1 and Th17 cell differentiation via IL-12 and/or IL-18 induction. FIGS. 4A-4F shows that RA PBMCs were treated with PBS (ctrl) or Syntenin-1 (SYN1; 1000 ng/mL) for 6 hours (qRT-PCR) or 24 hours (protein). Transcriptional regulation of Tbx21 (FIG. 4A), IFNγ (FIG. 4B), IL-18 (FIG. 4C), and IL-12 (FIG. 4D) was assessed by qRT-PCR (n=4-8). FIGS. 4E and 4F show that protein secretion of IL-12 (FIG. 4E) and IL-18 (FIG. 4F) was determined by ELISA (n=4-6). FIGS. 4G-4J show that RA PBMCs (FIGS. 4G to 4I) or negatively selected T cells (FIG. 4J) were supplemented with anti-CD3 and anti-CD28 (both 0.25 μg/mL) and were untreated (ctrl) or stimulated with LPS (100 ng/mL, +control), IL-12 (10 ng/mL, Th1 cells), IL-10, IL-6 and TGF-β (20 ng/mL and 4 ng/mL, respectively, Th17 cells) or syntenin-1 (1000 ng/mL) in the presence of absence of SDC-1 and IL-12 antibody (SDCab, IL-12ab) for 72 hours prior to determining the number of CD4+ IFNγ+ T cells (FIGS. 4G, 4I and 4J) or CD4+IL-17+ T cells by flow cytometry (FIG. 4H) (n=3-4). FIGS. 4K and 4L show that RA PBMCs were cultured with anti-CD3 and anti-CD28 (both 0.25 μg/mL) and were untreated (ctrl) or stimulated with LPS (100 ng/mL, +control), IL-12 (10 ng/mL, Th1 cells) or syntenin-1 (1000 ng/mL) alone or in combination with 2-DG (5 mM), mTORi (1 μM) and HIF1αi (2 μM) for 72 hours before determining the number of CD4+ IFNγ+ T cells (FIG. 4K) or CD4+IL-17+ T cells (FIG. 4L) by flow cytometry (n=5). Data are presented as mean±SEM; significant differences were determined by the Mann-Whitney test or one-way ANOVA. *p<0.05, **p<0.01, ***p<0.001, ****p<0.0001. ANOVA, analysis of variance.

FIGS. 5A-H show that local expression of Syntenin-1 advances arthritis in WT but not in SDC-1^{-/-} mice. FIGS. 5A to 5H show that wildtype (WT) and SDC-1^{-/-} C57BL/6 (SDC^{-/-}) mice were injected intra-articularly with adctrl (ctrl) or adSYN1 (3×10¹⁰ viral particles/ankle) on days 0, 7 and 14 and joint circumference (FIG. 5A) was monitored over 15 days (n=10 mice/group). On day 15, mice were sacrificed, and ankles were either used for histological analysis or qRT-PCR. FIGS. 5B and 5C show that sections from non-arthritic WT ctrl and WT or SDC^{-/-} mice injected with adSYN1 were stained for H&E (FIG. 5B) and scored on a 0-5 scale for synovial lining thickness, inflammation and bone erosion (FIG. 5C) (n=4). FIGS. 5D and 5E show that ankle sections from non-arthritic WT ctrl and WT or SDC^{-/-} mice injected with adSYN1 were stained for the macrophage markers F4/80, iNOS and arginase 1 (FIG. 5D) and subsequently scored on a 0-5 scale (FIG. 5E) (n=8-12). FIGS. 5F to 5H) show that ankles from non-arthritic WT ctrl and WT or SDC^{-/-} mice injected with 30 adSYN1 were homogenized and transcription levels of IRFs and iNOS (FIG. 5F), inflammatory cytokines (FIG. 5G), or prorepair factors (FIG. 5H) were quantified by qRT-PCR (n=6-7). Data are presented as mean±SEM; significant differences were determined by one-way ANOVA. *p<0.05, **p<0.01, ***p<0.001, ****p<0.0001. ANOVA, analysis of variance.

FIGS. 6A-I show that Syntenin-1 arthritic mice display hypermetabolic activity in wild-type mice which was mitigated in SDC-1^{-/-} animals. FIGS. 6A to 6I show that WT and SDC^{-/-} mice were injected intra-articularly with adctrl (ctrl) or adSYN1 (3×10¹⁰ viral particles/ankle) on days 0, 7 and 14. FIG. 6A shows that ankles from non-arthritic WT ctrl (day 0) and WT adSYN1 mice (day 15) were homogenized and expression of glycolytic proteins, GLUT1, HK2, mTOR/p70 and LDHA was determined by Western blot analysis and β-actin served as a loading control. FIGS. 6B to 6E show that ankles from non-arthritic WT ctrl and WT or SDC^{-/-} mice injected with adSYN1 were homogenized and transcriptional regulation of the glycolytic factors GLUT1 (FIG. 6B), HIF1α (FIG. 6C), cMYC (FIG. 6D), and LDHA (FIG. 6E) was determined by qRT-PCR (n=5-9). FIGS. 6F and 6G show that ankles from non-arthritic WT ctrl and WT or SDC^{-/-} mice injected with adSYN1 were stained for GLUT1, HIF1α, cMYC and mTOR/p70 (FIG. 6F) and their staining was scored on a 0-5 scale (FIG. 6G) (n=8). FIGS. 6H and 6I show that mRNA levels of PPARγ (FIG. 6H) and AMPK (FIG. 6I) were quantified in joints from non-arthritic WT ctrl and WT adSYN1 or SDC^{-/-} adSYN1 mice by qRT-PCR (n=8-12). Data are presented as mean±SEM; significant differences were determined by one-way ANOVA. *p<0.05, **p<0.01, ***p<0.001, ****p<0.0001. ANOVA, analysis of variance.

FIGS. 7A-O show that RA preosteoclasts and arthritic joint cells are transformed into mature osteoclasts by syntenin-1. FIGS. 7A to 7B show that WT and SDC^{-/-} mice were injected intra-articularly with adctrl (ctrl) or adSYN1 (3×10¹⁰ viral particles/ankle) on days 0, 7 and 14. Ankles from non-arthritic WT ctrl and WT or SDC^{-/-} mice injected with adSYN1 were stained for T cell marker CD3 (FIG. 7A) and scored on a 0-5 scale (FIG. 7B) (n=4). FIGS. 7C to 7E show that ankles from non-arthritic WT ctrl and WT or SDC^{-/-} mice injected with adSYN1 were homogenized and transcription levels of IL-17 and IFNγ (FIG. 7C), IL-18 (FIG. 7D), and IL-12 (FIG. 7E) were quantified by qRT-PCR (n=4-10). FIGS. 7F and 7G) show that the transcript levels of blood syntenin-1 (FIG. 7F, n=67) or synovial tissue SDC-1 (FIG. 7G, n=87) determined by RNAseq18 were correlated against bone erosion as determined by radiographic images of hands and feet by Sharp/van der Heijde score. FIG. 7H show that, in the presence of M-CSF and RANKL (10 ng/mL each, suboptimal condition). RA monocytes were differentiated into preosteoclasts for 7 days, stimulated with PBS (ctrl) or syntenin-1 (1000 ng/mL) for 6 hours and osteoclastic factors were assessed by qRT-PCR (FIG. 7H) (n=7). FIGS. 7I and 7J show that ankles from non-arthritic WT ctrl and WT or SDC^{-/-} mice injected with adSYN1 were stained for TRAP (FIG. 7I) and TRAP+ cells (FIG. 7J) and were quantified at ×100 magnification (n=4). FIGS. 7K-7O show that ankles from non-arthritic WT ctrl and WT or SDC^{-/-} mice injected with adSYN1 were homogenized and analyzed for transcriptional regulation of TRAP (FIG. 7K), RANK (FIG. 7L), RANKL (FIG. 7M), CTSK (FIG. 7N), and NFATc1 (FIG. 7O) by qRT-PCR (n=4-12). Data are presented as mean±SEM; significant differences were determined by one-way ANOVA. *p<0.05, **p<0.01, ***p<0.001, ****p<0.0001. ANOVA, analysis of variance.

FIG. 8 shows syntenin-1 advances glycolytic reprogramming in RA CD14+CD86+ GLUT1+MΦs and murine F4/80+ iNOS+MΦs. Syntenin-1 reconfigures naive cells into metabolic RA CD14+CD86+ GLUT1+MΦs that display a broad array of glycolytic factors together with impaired oxidative intermediates through SDC-1 ligation, glucose uptake, and/or mTOR signaling. In Syntenin-1-induced arthritis, F4/80+ iNOS+MΦs recapitulate glycolytic RA myeloid cell mechanism of function, by expanding the inflammatory and glycolytic imprints which are dysregulated in SDC-1^{-/-} animals. Both in RA cells and/or experimental models, mTOR-driven MΦ glycolytic reprogramming and their crosstalk with Th1 cells via IL-12 escalation are responsible for Syntenin-1-induced arthritogenicity. RA, rheumatoid arthritis.

FIGS. 9A-M show that in RA MΦs, Syntenin-1 shifts oxidative phosphorylation to glycolytic activity. FIG. 9A shows that to titrate the optimal dose of Syntenin-1, RA monocyte-differentiated MΦs were treated with Syntenin-1 (0, 100, 300, 500, 1000, and 1500 ng/ml) for 24 h and levels of TNFα were determined by ELISA. FIG. 9B shows that sera from NL and RA were analyzed for the Syntenin-1 protein expression by ELISA (n=5). FIG. 9C shows that RA MΦs were treated with PBS (ctrl) or Syntenin-1 (SYN1; 1000 ng/ml) for 6 h before quantifying IFNα and IFNβmRNA. FIG. 9D show that synovial tissue from RA patients was fluorescently stained for the expression of SDC-1, and Syntenin-1 in CD68+ cells in the presence of DAPI. FIGS. 9E to 9J show that RA MΦs were treated with PBS or Syntenin-1 (1000 ng/ml) and transcription of GLUT1 (FIG. 9E), RAPTOR (FIG. 9F), HK2 (FIG. 9G), PFK2 (FIG. 9H), PDK1 (FIG. 9I), and PKM2 (FIG. 9J) was assessed by qRT-PCR (n=6). FIG. 9K show that RA monocyte-differentiated MΦs were untreated or stimulated with Syntenin-1 (1000 ng/ml) for 72 h before determining the number of CD14+CD206+ GLUT1+MΦs by flow cytometry (n=4). FIG. 9L show that exemplary gating strategy of FIGS. 3H and 3I. FIG. 9M shows that RA MΦs were treated with PBS or Syntenin-1 (1000 ng/ml) in the presence or absence of SDC Ab (SDCab; 1:100) and transcription of oxidative enzymes ACO2, OGDH, SDH2, FH, and MDH2 was assessed by qRT-PCR (n=6). Data are presented as mean±SEM; significant differences were determined by the Mann-Whitney test or one-way ANOVA. *p<0.05, **p<0.01, ***p<0.001, ****p<0.0001.

FIGS. 10A-B show that authenticating local expression of Syntenin-1 in Syntenin-1-induced arthritis model and gating strategy in RA Th1 and Th17 differentiation. FIG. 10A shows that the Syntenin-1 transcription level was detected by real-time RT-PCR in wild-type mice intra-articularly injected with adenovirus control (ctrl) or Ad-Syntenin-1 (adSYN1, 3×10¹⁰ viral particles/ankle) on day 15 post-onset. FIG. 10B show that RA PBMCs were supplemented with anti-CD3 and anti-CD28 (both 0.25 μg/ml) and were untreated (ctrl) or stimulated with LPS (100 ng/ml, +control), IL-12 (10 ng/ml, Th1 cells), IL-1, IL-6, and TGF-β (20 ng/ml and 4 ng/ml, respectively, Th17 cells) or Syntenin-1 (1000 ng/ml) for 72 h prior to determining the number of CD4+ IFNγ+ T cells or CD4+IL-17+ T cells by flow cytometry (n=4). Visualization of an exemplary gating strategy of FIGS. 4G and 4H.

FIGS. 11A-J show Syntenin-1 reprogrammed endothelial cells display a robust inflammatory phenotype. Relative expression of Syntenin-1 (FIG. 11A) or SDC-1 (FIG. 11B) was determined by RNAseq (Lewis M J, et al. Cell reports. 2019; 28:2455-2470 e2455) in synovial tissue biopsies from RA non-responsive (Non-R)(ΔDAS28≤0.6, n=16-23) and moderate (ΔDAS28 1.2 & >0.6, n=21) and good responsive (ΔDAS28>1.2, n=35-50) patients. FIG. TIC shows RA STs were fluorescently stained to authenticate the colocalization of SDC-1 and Syntenin-1 on VWF⁺ endothelial cells in the presence or absence of DAPL (n=3, original magnification×20). FIG. 11D shows HUVECs were treated with Syntenin-1 (1000 ng/ml) for 0-60 mins and phosphorylation of ERK, p-38, JNK, AKT, STAT1, STAT3, and degradation of IκBα was determined by Western blot analysis and actin served as a loading control, n=3. HUVECs were treated with PBS (ctrl) or Syntenin-1 (1000 ng/ml) for 6 h and transcription of IRFs (FIG. 11E), inflammatory mediators (FIG. 11F), TLRs (FIG. 11I), and pro-repair factors (FIG. 11J) were assessed by qRT-PCR (n=7-12). HUVECs were treated with PBS or Syntenin-1 (1000 ng/ml) in the presence or absence of SDC-1 Ab (SDCab; 1:100), IL-5R Ab (IL5Ra; 2 μg/ml), or PDZli (PDZ1; 10 μM) for 6 h before quantifying transcription levels of TNFα (FIG. 11G) and IL-1β (FIG. 11H), (5-7). Data are presented as mean±SEM; significant differences were determined by the Mann-Whitney test, 2way ANOVA, or one-way ANOVA. *p<0.05, **p<0.01, ***p<0.001, ****p<0.0001.

FIGS. 12A-F show that Syntenin-1 ligation to SDC-1 promotes endothelial cell migration and induction of proangiogenic factors from these cells. FIG. 12A shows that a scratch was created in the middle of the wells that contained confluent HUVECs. Thereafter, cells were either untreated (PBS) or stimulated with Syntenin-1 (1000 ng/ml) or 10% FBS as a positive control for 24 h. In parallel, cells were treated with SDC1-Ab (1:100), IL-5R Ab (2 μg/ml) or PDZ1i (10 μM), mTORi (1 μM), and HIF1αi (2 μM) for 24 h, (n=3). FIG. 12B shows the number of cells in the scratch area was counted and compared to the untreated control, (n=3). HUVECs were treated with PBS (ctrl) or Syntenin-1 (1000 ng/ml) for 6 h and transcription of bFGF, VEGF, IL-18, FGFR2, VEGFR1, VEGFR2, IL-18R (FIG. 12C), and CXCL1, CXCL5, CXCR2 (FIG. 12D) or DLL1, DLL4. JAG1, JAG2, Notch1 (FIG. 12E) was determined by qRT-PCR, (n=6-10). HUVECs were treated with PBS or Syntenin-1 (1000 ng/ml) in the presence or absence of SDC-1 Ab (SDCab; 1:100), IL-5R Ab (IL5Ra; 2 μg/ml), or PDZli (PDZ1; 10 μM) for 6 h before quantifying transcription levels of DLL4 (FIG. 12F), (n=6). Data are presented as mean±SEM; significant differences were determined by the Mann-Whitney test, 2way ANOVA, or one-way ANOVA. *p<0.05, **p<0.01, ***p<0.001, ****p<0.0001.

FIGS. 13A-L show that Syntenin-1 reprogrammed endothelial cells display accelerated glycolytic activity with no effect on oxidative phosphorylation. HUVECs were treated with PBS (ctrl) or Syntenin-1 (1000 ng/ml) for 6 h and transcription of glycolytic factors including GLUT1, HK2, PFK2, PKM2, HIF1α, cMYC, RAPTOR (FIG. 13A), Lactate receptor (GPR81) and sensors (MCT1/4)(FIG. 13D) as well as oxidative metabolites (AMPK, PGC-1a) (FIG. 13I), (n=7-10) were determined by qRT-PCR. HUVECs were treated with 20 Syntenin-1 (1000 ng/ml) for 0-60 mins to detect expression of GLUT1. HK2. PFK2, cMYC. HIF1α, and LDHA (FIG. 13B) or 0-48 h to detect HK2, PFK2, mTOR/RAPTOR and LDHA (FIG. 13C) and actin was visualized as equal loading, (n=3). HUVECs were treated with PBS or Syntenin-1 (1000 ng/ml) in the presence or absence of 2-DG (5 mM), cMYCi (50 μM) and/or mTORi (1 μM) or HIF1αi (2 μM) to quantify transcription of HIF1α (FIG. 13E, n=10), RAPTOR (FIG. 13F, n=7). TNF (FIG. 13H, n=5). FIG. 13G shows that using a Seahorse XF Glycolysis Stress Test Kit from Agilent (cat #103020-100), ECAR was evaluated in HUVECs treated with PBS and Syntenin-1 for 0-112 min and data are shown as Glycolysis and Glycolytic Capacity, (n=6). HUVECs were treated with PBS or Syntenin-1 (1000 ng/ml) in the presence or absence of SDC-1 Ab (SDCab: 1:100). IL-5R Ab (IL5Ra; 2 μg/ml), PDZli (PDZ1; 10 μM), HIF1αi (2 μM) or mTORi (1 μM) for 24 h before measuring pyruvate (FIG. 13J) and citrate (FIG. 13K) levels by colorimetric assay, (n=5). FIG. 13L shows that employing a Seahorse XF Glycolysis Stress Test Kit from Agilent (cat #103020-100), OCR was evaluated in HUVECs treated with PBS and Syntenin-1 for 0-112 min and data are shown as ATP production, (n=5). Data are presented as mean±SEM; significant differences were determined by the Mann-Whitney test, 2way ANOVA, or one-way ANOVA. *p<0.05. **p<0.01, ***p<0.001, ****p<0.0001.

FIGS. 14A-L show the inflammatory profile surpasses the pro-repair phenotype in Syntenin-1 reprogrammed RA FLS. FIG. 14A shows RA STs were fluorescently stained to authenticate the colocalization of SDC-1 and Syntenin-1 expression on Vimentin⁺ FLS in the presence or absence of DAPI, (n=3, original magnification×20). FIG. 14B show RA FLS were treated with Syntenin-1 (1000 ng/ml) for 0-60 mins and phosphorylation of AKT, STAT1, STAT3, Src, p38, or degradation of IκBα was determined by Western blot analysis and actin served as a loading control, (n=3). RA FLS were treated with PBS (ctrl) or Syntenin-1 (1000 ng/ml) for 6 h and transcription of IRFs (FIG. 14C, n=4), inflammatory mediators (FIGS. 14D-E, n=4-8), TLRs (FIG. 14J, n=4) and pro-repair factors (FIG. 14K, n=6-8) were evaluated by qRT-PCR. RA FLS were treated with PBS or Syntenin-1 (1000 ng/ml) in the presence or absence of SDC-1 Ab (SDCab; 1:100), IL-5R Ab (IL5Ra; 2 μg/ml), or PDZ1i (PDZ1; 10 μM) for 24 h or 6 h before quantifying production levels of TNFα, (FIG. 14F, n=5), IL-8 (FIG. 14G, n=5), IL-12 (FIG. 141L n=4) or transcription of inflammatory (FIG. 14H, n=5-9) or pro-repair mediators (FIG. 14L, n=7-8) were evaluated by ELISA or real-time RT-PCR. Data are presented as mean f SEM; significant differences were determined by the Mann-Whitney test, 2way ANOVA, or one-way ANOVA. *p<0.05, **p<0.01, ***p<0.001, ****p<0.0001.

FIGS. 15A-P show RA FLS reprogrammed by Syntenin-1 displays dysregulated mitochondrial oxidative stress. RA FLS were treated with PBS (ctrl) or Syntenin-1 (1000 ng/ml) for 6 h and transcription of glycolytic mediators GLUT1, HK2, PFK2, cMYC, RAPTOR (FIG. 15A, n=8) and LDHA, LDHB (FIG. 15D, n=4) as well as oxidative metabolites SIRT1, SIRT3, SIRTS (FIG. 15L, n=4) or AMPK (FIG. 15M, n=8) and HIF1a (FIG. 15N, n=8) were determined by qRT-PCR. RA FLS were treated with Syntenin-1 (1000 ng/ml) for 0-48 h to detect HK2, LDHA, RAPTOR (FIG. 15B, n=3) or 0-60 min to detect AMPK, HIF1α or RAPTOR protein expression (FIG. 15O, n=3) and actin was considered as equal loading by Western blotting. RA FLS were treated with PBS or Syntenin-1 (1000 ng/ml) in the presence or absence of SDC-1 Ab (SDCab; 1:100), IL-5R Ab (IL5Ra; 2 μg/ml), or PDZli (PDZ1; 10 μM) for 6 h before quantifying transcription levels of RAPTOR (FIG. 15C) or AMPK (FIG. 15P), (n=4). RA FLS were treated with PBS or Syntenin-1 (1000 ng/ml) and lactate levels were quantified after 24 h or RA FLS were treated with PBS or Syntenin-1 (1000 ng/ml) in the presence or absence of SDC-1 Ab (SDCab; 1:100), IL-5R Ab (IL5Ra; 2 μg/ml), PDZli (PDZ1; 10 μM), mTORi (1 μM), HIF1αi (2 μM) and cMYCi (50 μM) and pyruvate, citrate, and succinate protein levels were measured after 24 h by colormetric assay, (FIGS. 15F-H, n=3-7). RA FLS were treated with PBS (basal) or Syntenin-1 (1000 ng/ml, induced), and total ATP, glycoATP, and mitoATP (FIGS. 15I-K) were determined by Seahorse XF Real-Time ATP Rate Assay Kit (n=13). Data are presented as mean z SEM; significant differences were determined by the Mann-Whitney test, 2way ANOVA, or one-way ANOVA. *p<0.05, **p<0.01, ***p<0.001, ****p<0.0001.

FIGS. 16A-M show Syntenin-1 rewired RA FLS exhibits mitochondrial fusion and fission, in addition, the inflammatory phenotype was differentially regulated compared to RA FLS migration in response to Syntenin-1. RA STs were fluorescently stained to authenticate the colocalization of Mitofusin (MFN2) (FIGS. 16A-B) and DRP1 (FIGS. 16C-D) expression on Vimentin⁺ FLS in the presence or absence of DAPI, (n=3, original magnification×60 or 500). RA FLS were treated with Syntenin-1 (1000 ng/ml) for 0-60 min to detect Mitofusin-2 and DRP1 expression (FIG. 16E, n=3) and actin served as equal loading, (n=3). RA FLS were treated with Syntenin-1 (1000 ng/ml) in the presence or absence of HIF1αi (2 μM) for 6 h to determine transcription of IL-1β (FIG. 16F, n=6), IL-6 (FIG. 16G, n=5), IL-8 (FIG. 16H, n=5) and CCL2 (I, n=6) by real-time RT-PCR. A scratch was created in the middle of the wells that contained confluent RA FLS. Thereafter, cells were either untreated (PBS) or stimulated with Syntenin-1 (1000 ng/ml) or bFGF (100 ng/ml) as a positive control for 24 h. In parallel, cells were treated with SDC1-Ab (1:100), IL-5R Ab (2 g/ml) or PDZli (10 μM), HIF1αi (2 μM), and mTOR1i (1 μM) for 24 h, (J, n=4). The number of cells in the scratch area was counted and compared to the untreated control, (K, n=4). RA FLS were treated with PBS or Syntenin-1 (1000 ng/ml) in the presence or absence of SDC-1 Ab (SDCab: 1:100), IL-5R Ab (IL5Ra; 2 μg/ml), or PDZli (PDZ1; 10 μM), and/or HIF1αi (2 μM) or mTORi (1 PM) for 6 h before quantifying transcription levels of VEGF (FIG. 16L, n=5) and Notch1, FGF2, CXCL1, CXCL5 (FIG. 16K or 16M, n=8 or 7). Data are presented as mean±SEM; significant differences were determined by the Mann-Whitney test, 2way ANOVA, or one-way ANOVA. *p<0.05, **p<0.01, ***p<0.001, ****p<0.0001.

FIGS. 17A-I show Syntenin-1 arthritic mice recapitulate RA pathology by exhibiting Vimentin ‘fibroblast and VWF’ endothelial cell recruitment in WT mice which was mitigated in SDC-1^{-/-} animals. FIG. 17A shows WT and SDC^{-/-} mice were injected intra-articularly with ad-ctrl (ctrl) or adSYN1 (3×10¹⁰ viral particles/ankle) on days 0, 7, and 14 and joint circumference was monitored over 15 days (n=10 mice/group). Ankles from non-arthritic WT ctrl and WT or SDC^{-/-} mice injected with adSYN1 were stained for Vimentin and VWF (FIGS. 17B, 17C-D) or VEGFR2 and Notch1 (FIGS. 17E, 17F-G) and their staining was scored on a 0-5 scale (n=4). Ankles from non-arthritic WT ctrl and WT or SDC^{-/-} mice injected with adSYN1 were homogenized and transcriptional regulation of VEGFR1 and Notch1 (FIG. 17H) or RAPTOR, and HIF1α (FIG. 17I) was determined by qRT-PCR (n=4-6). Data are presented as mean±SEM; significant differences were determined by the Mann-Whitney test, 2way ANOVA, or one-way ANOVA. *p<0.05, **p<0.01, ***p<0.001, ****p<0.0001.

FIGS. 18A-H show that VEGFR2. Notch1, RAPTOR, and HIF1α are represented in RA ST FLS and endothelial cells. RA STs were fluorescently stained to authenticate the colocalization of VEGFR2 (FIGS. 18A, 18C), Notch1 (FIGS. 18B, 18D), mTOR1 (FIGS. 18E, 18G), and HIF1α (FIGS. 18F, 18H) on Vimentin*FLS and VWF⁺ endothelial cells in the presence or absence of DAPI, (n=3, original magnification×20).

FIGS. 19A-R show Syntenin1-induced metabolic activity fine-tunes transcription of angiogenic and inflammatory factors in RA ST explants. Expression levels of Syntenin-1 (FIG. 19A), SDC-1 (FIG. 19B), HIf1α (FIG. 19C) and Raptor (FIG. 19D) are displayed on the lining and sublining RA FLS as well as endothelial cells based on single-cell RNA sequencing data from Wei et al. (Wei K, et al. Nature. 2020; 582:259-264). FIG. 19E shows a representative RA ST utilized in FIGS. 19F-R. Thirty milligrams of RA ST were cut into small pieces to allow proper access to stimuli and were starved o/n in 0% FBS RPMI with or without SDC-1-Ab (1:100), mTOR1i (1 μM), and HIF1αi (2 μM). RA STs were stimulated with 1000 ng/ml Syntenin-1 for 6-8 h. Synovial tissues were harvested for transcriptome analysis by qRT-PCR and supernatants were used for protein quantification by ELISA. The transcription level of JAG1 (FIG. 19F), Notch1 (FIG. 19G), VEGF (FIG. 19H), VEGFR1 (FIG. 19I), and RAPTOR (FIG. 19J) was quantified by real-time RT-PCR, n=6-8. RA explants were untreated or stimulated with 5000 ng/ml Syntenin-1 in the presence or absence of SDC1-Ab (1:100), HIF1αi (2 μM), or mTOR1i (IμM) for 6-8 h. The transcription levels of RAPTOR (FIG. 19K, n=3), VEGF (FIG. 19L, n=5), IL-1β (FIG. 19M, n=8), CCL5 (FIG. 19N, n=6), IL-6 (FIG. 19O, n=6), IL-8 (FIG. 19P, n=6), CCL2 (FIG. 19Q, n=8) were evaluated by qRT-PCR or ELISA. FIG. 19R shows the production of TNFα evaluated by ELISA, (n=10). Data are presented as mean±SEM: significant differences were determined by the Mann-Whitney test, 2way ANOVA, or one-way ANOVA. *p<0.05, **p<0.01, ***p<0.001, ****p<0.0001.

FIGS. 20A-F show blood Syntenin-1 and SDC-1 relative levels are unaffected by RA therapy and glycolytic metabolites can modulate inflammatory factors and oxidative metabolites in Syntenin-1 reprogrammed endothelial cells. Relative expression of Syntenin-1 (FIG. 20A) or SDC-1 (FIG. 20B) was determined by RNAseq (Lewis M J, et al. Cell reports. 2019; 28:2455-2470 e2455) in blood from RA non-responsive (Non-R)(ΔDAS28≤0.6, n=16) or those with moderate (ΔDAS28≤1.2 & >0.6, n=22) or good response (ΔDAS28>1.2, n=21). HUVECs were treated with PBS (ctrl) or Syntenin-1 (1000 ng/ml) for 6 h and transcription of LDHA and LDHB (FIG. 20C, n=8) were assessed by qRT-PCR. HUVECs were treated with PBS or Syntenin-1 (1000 ng/ml) in the presence and/or absence of or cMYCi (5 μM) and/or 2-DG (5 mM), mTORi (1 μM), HIF1αi (2 μM) to quantify transcription of GLUT1 (FIG. 20D, n=4-9) after 6 h using qRT-PCR and to assess secretion of citrate (FIG. 20E, n=3) or pyruvate (FIG. 20F, n=4) following 24 h stimulation with colorimetric assay. Data are presented as mean±SEM; significant differences were determined by the Mann-Whitney test, 2way ANOVA, or one-way ANOVA. *p<0.05. **p<0.01, ***p<0.001, ****p<0.0001.

FIGS. 21A-I show RA FLS reprogrammed by Syntenin-1 displays an expanded glycolytic landscape. RA FLS were treated with PBS (ctrl) or Syntenin-1 (1000 ng/ml) for 6 h and transcription of glycolytic mediators, GLUT1 (FIG. 2IA, n=8). HK2 (FIG. 2IB, n=9), PFK2 (FIG. 2IC, n=8), cMYC (FIG. 2ID, n=8), RAPTOR (FIG. 2IE, n=9). PKM2 (FIG. 21F, n=4), Notch, FGF2, CXCL1, and CXCL5 (FIG. 2I1, n=8) were assessed by qRT-PCR. RA STs were fluorescently stained to authenticate the colocalization of Mitofusin (MFN2) (FIG. 2IG) and DRP1 (FIG. 2IH) expression on Vimentin⁺ FLS in the presence or absence of DAPL (n=3, original magnification×20). Data are presented as mean+SEM: significant differences were determined by the Mann-Whitney test, 2way ANOVA, or one-way ANOVA. *p<0.05, **p<0.01, ***p<0.001, ****p<0.0001.

FIG. 22 shows that the Syntenin-1/SDC-1 pathway influences RA FLS and endothelial cell pathology in RA explants and experimental models. The schematic figure demonstrates the mechanism by which Syntenin-1 reprograms endothelial cells and RA FLS and how inflammatory and angiogenic markers are impacted by SDC-1 and metabolic intermediates.

FIGS. 23A-C show that syntenin-1 reprogrammed endothelial cells display a robust inflammatory phenotype. FIG. 23A shows HUVECs were treated with PBS (ctrl) or Syntenin-1 (1000 ng/ml) in the presence or absence of SDC-1 Ab (SDCab; 1:100), IL-5R Ab (IL5Ra; 2 μg/ml), or PDZli (PDZ1; 10 μM) for 6 h or 24 h and transcription or translation levels of IL-1β (FIG. 23A), TNFα (FIG. 23B), and CCL5 (FIG. 23C) was assessed by qRT-PCR and/or ELISA (n=5-12). Data are presented as mean±SEM; significant differences were determined by the Mann-Whitney test, 2way ANOVA, or one-way ANOVA. *p<0.05, **p<0.01, ***p<0.001, ****p<0.0001.

FIGS. 24A-B show the inflammatory profile surpasses the pro-repair phenotype in Syntenin-1 reprogrammed RA FLS. RA FLS were treated with PBS (ctrl) or Syntenin-1 (1000 ng/ml) in the presence or absence of SDC-1 Ab (SDCab; 1:100), IL-5R Ab (IL5Ra; 2 μg/ml), or PDZ1i (PDZ1; 10 μM) for 6 h or 24 h before quantifying transcriptional or translational levels of inflammatory mediators (FIG. 24A, n=4-9), and pro-repair factors (FIG. 24B, n=4) by qRT-PCR or ELISA. Data are presented as mean±SEM; significant differences were determined by the Mann-Whitney test, 2way ANOVA, or one-way ANOVA. *p<0.05, **p<0.01, ***p<0.001, ****p<0.0001.

FIGS. 25A-B show syntenin-1 arthritic mice recapitulate RA pathology by exhibiting Vimentin⁺fibroblast and VWF⁺ endothelial cell recruitment in WT mice which was mitigated in SDC-1^{-/-} animals. WT and SDC-1^{-/-} mice were injected intra-articularly with ad-ctrl (ctrl) or adSYN1 (3×10¹⁰ viral particles/ankle) on days 0, 7, and 14 and joint circumference was monitored over 15 days (n=10 mice/group). Ankles from non-arthritic WT ctrl and WT or SDC-1^{-/-} mice injected with ad-SYN1 were stained for H&E, Vimentin, and VWF (FIG. 25A) and their staining was scored on a 0-5 scale (FIG. 25B, n=4-9). Data are presented as mean±SEM; significant differences were determined by the Mann-Whitney test, 2way ANOVA, or one-way ANOVA. *p<0.05, **p<0.01, ***p<0.001, ****p<0.0001.

FIGS. 26A-G show blood Syntenin-1 and SDC-1 relative levels are unaffected by RA therapy and glycolytic metabolites can modulate inflammatory factors and oxidative metabolites in Syntenin-1 reprogrammed endothelial cells. Relative expression of Syntenin-1 (FIGS. 26A and C) or SDC-1 (FIGS. B and D) was determined by RNAseq in RA STs from patients treated with anti-TNF therapy (Certolizumab, n=27) or anti-IL6R Ab (Tocilizumab, n=44) in non-responsive and/or those with moderate or good response. Endothelial cells were untreated (ctrl) or treated with Syntenin-1 for 24 h, before cells were stained with VEGFR, or DAPI (FIG. 26E) and quantified by MFI in 17-22 HPFs (FIG. 26F). Alternatively, endothelial cells were untreated or stimulated by Syntenin-1 from 6-24 h and lysates were assessed for Notch1 or β-actin (FIG. 26G, n=3). Data are presented as mean SEM; significant differences were determined by the Mann-Whitney test, 2way ANOVA, or one-way ANOVA. *p<0.05, **p<0.01, ***p<0.001, ****p<0.0001.

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present disclosure is not entitled to antedate such publication by virtue of prior disclosures. Further, the dates of publication provided herein can be different from the actual publication dates, which can require independent confirmation.

### Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise.

The use of the word “a” or “an” when used in conjunction with the term “comprising” in the claims and/or the specification may mean “one,” but it is also consistent with the meaning of “one or more,” “at least one,” and “one or more than one.”

“Optional” or “optionally” means that the subsequently described event, circumstance, or material may or may not occur or be present, and that the description includes instances where the event, circumstance, or material occurs or is present and instances where it does not occur or is not present.

The word “or” as used herein means any one member of a particular list and also includes any combination of members of that list. The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the standard deviation of error for the device or method being employed to determine the value.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. “Inhibit,” “inhibiting” and “inhibition” mean to diminish or decrease an activity, level, response, condition, disease, or other biological parameter. This can include, but is not limited to, the complete ablation of the activity, response, condition, or disease. This may also include, for example, a 10% inhibition or reduction in the activity, response, condition, or disease as compared to the native or control level. Thus, in some aspects, the inhibition or reduction can be a 10, 20, 30, 40, 50, 60, 70, 80, 90, 100%, or any amount of reduction in between as compared to native or control levels. In some aspects, the inhibition or reduction is 10-20, 20-30, 30-40, 40-50, 50-60, 60-70, 70-80, 80-90, or 90-100% as compared to native or control levels. In some aspects, the inhibition or reduction is 0-25, 25-50, 50-75, or 75-100% as compared to native or control levels.

“Treatment” and “treating” refer to administration or application of a therapeutic agent (e.g., syndecan-1 inhibitor or syntenin-1 inhibitor) to a subject or performance of a procedure or modality on a subject for the purpose of obtaining a therapeutic benefit of a disease or health-related condition. For example, a treatment may include administration of a pharmaceutically effective amount of a syndecan-1 inhibitor or a syntenin-1 inhibitor, or a combination thereof.

As used herein, the term “treating” refers to partially or completely alleviating, ameliorating, relieving, delaying onset of, inhibiting or slowing progression of, reducing severity of, and/or reducing incidence of one or more symptoms or features of a particular disease, disorder, and/or condition (e.g. rheumatoid arthritis or synovial inflammation). Treatment can be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition. For example, the disease, disorder, and/or condition can be rheumatoid arthritis or synovial inflammation.

As used herein, the term “subject” refers to the target of administration, e.g., a human. Thus, the subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In some aspects, a subject is a mammal. In another aspect, a subject is a human. In some aspects, a subject is a non-human primate. The term does not denote a particular age or sex. Thus, adult, child, adolescent and newbom subjects, as well as fetuses, whether male or female, are intended to be covered.

As used herein, the term “patient” refers to a subject afflicted with a condition, disease or disorder (e.g., rheumatoid arthritis or synovial inflammation). The term “patient” includes human and veterinary subjects. In some aspects of the disclosed methods, the “patient” has been diagnosed with rheumatoid arthritis or synovial inflammation. In some aspects of the disclosed methods, the “patient” has been diagnosed with a need for treatment (e.g. treatment for rheumatoid arthritis or preventing the development of rheumatoid arthritis), such as, for example, prior to the administering step.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Melanoma differentiation-associated gene-9 (MDA-9) or Syntenin-1 is an adaptor-like molecule that was identified as a binding partner of syndecan-1 (SDC-1) (Das S K, et al. Adv Cancer Res 2019; 144:137-191). Syntenin-1 was first cloned and described as a tumorigenic factor in melanoma cell lines in response to IFNβ and mezerein (Lin J J, et al. Gene 1998; 207:105-110). In cancer patients, SDC-1 and IL-5R regulate the function of Syntenin-1 through their interaction with the protein-interaction domain, PDZ2, however, they do not bind to the PDZ1 domain (Shimada T, et al. Int J Mol Sci 2019; 20). These findings highlight that PDZ2 is the primary active domain of Syntenin-1/SDC-1 signaling. Additionally, the PDZ2 domain of Syntenin-1 interacts with SRC and FAK, and pharmacological or genetic dysregulation of SRC nullifies Syntenin-1-mediated growth in the human melanoma metastasis model in vivo (Boukerche H, et al. Proc Natl Acad Sci USA 2008; 105:15914-15919; and Boukerche H, et al. Oncogene 2010; 29:3054-3066). Distinctly, the PDZ1 domain functions as a docking site for TGFβ (Menezes M E, et al. Oncotarget 2016; 7:80175-80189). EGFR (Dasgupta S, et al. Clin Cancer Res 2013; 19:4621-4633), and IGF1R (Das S K, et al. Cancer Res 2018: 78:2852-2863) in cancer cells exposed to Syntenin-1. Extending these observations, PDZ1 inhibitors can specifically target Syntenin-1 and EGFR interaction in preclinical models of glioblastoma multiforme (Das S K, et al. ACS Chem Neurosci 2019; 10:1121-1123).

Earlier studies have documented that Syntenin-1 expands tumor-cell migration, invasion, and metastasis: conversely, this function is impaired in knockout mice (Das S K, et al. Oncotarget 2016; 7:46848-46861). Corroborating these findings, patients with melanoma liver metastases exhibit Syntenin-1 overexpression compared to non-metastasizing counterparts (Boukerche H, et al. mda-9/Syntenin: a positive regulator of melanoma metastasis. Cancer Res 2005; 65:10901-10911; and Boukerche H, et al. Cancer Res 2007; 67:1812-1822). Expanding these findings, activation of SRC and MAPK by Syntenin-1 is responsible for NF-κB signaling that advances melanoma cell migration and invasion in part through MMP2/MMP9 modulation (Boukerche H, et al. Proc Natl Acad Sci USA 2008; 105:15914-15919; and Boukerche H, et al. Oncogene 2010; 29:3054-3066). On the contrary, others found that Syntenin-1 disrupted IL-1− and LPS-induced NF-cB activation and IL-8 transcription in 293 cells (Chen F, et al. Cell Signal 2008; 20:666-674). It was further demonstrated that in IL-1-stimulated cells, Syntenin-1 interacts with TRAF6 by displacing the IRAK1 association (Chen F, et al. Cell Signal 2008; 20:666-674).

Although both PDZ1 and PDZ2 are postulated to be involved in signaling directed by Syntenin-1, more recent evidence reveals that PDZ2 is indispensable for SRC and NF-cB activation in human melanoma cells (Boukerche H, et al. Oncogene 2010; 29:3054-3066). Nonetheless, Syntenin-1-activated signaling pathways or interacting partners are cell-type-specific. In keeping with this concept, Syntenin-1 amplifies PI3K/AKT or STAT3 signaling to exacerbate metastasis in small cell lung cancer (Kim W Y, et al. Exp Mol Med 2014; 46:e90) or prostate cancer (Das S K, et al. Cancer Res 2018; 78:2852-2863), respectively, rather than NF-κB activation.

In the lung metastatic model, the inflammatory imprint of Syntenin-1 is expanded by monokines that promote Th17 cell polarization (Das S K, et al. Oncotarget 2016; 7:46848-46861). While the significance of Syntenin-1 is well described in cancer its cellular origin, immunoregulation, and molecular mechanism are completely unknown in rheumatoid arthritis (RA). The results described here demonstrate that Syntenin-1 is highly enriched in RA compared to normal (NL) synovial tissue (ST), where it colocalizes with SDC-1 on the CD14¹ macrophages (MΦs). Consistently. RNAseq analysis exhibits that Syntenin-1 and SDC-1 transcriptome is closely linked to CD68⁺ MΦ frequency in RA STs. Syntenin-1 and SDC-1 levels were mutually potentiated by LPS/IFNγ stimulation in myeloid cells. However, because Syntenin-1 and SDC-1 expression were unaffected by the standard of care monotherapies, their mechanism of function was uncovered in RA patients and the preclinical model.

As disclosed herein, Syntenin-1 reprograms naïve cells into inflammatory RA MΦs that express a broad range of interferon transcription factors (e.g., IRF1/7/8/9) and monokines (e.g., IL-1β, TNF-α, IL-6, IL-8, and CCL2) that are exclusively impaired by an SDC-1 antibody but not by blockade of IL-5R or PDZ1 pathways. In parallel with its inflammatory phenotype, metabolic reprogramming of RA CD14⁺CD86⁺GLUT1⁺MΦs by Syntenin-1 is heavily dependent on glucose uptake and mTOR signaling. Concurrently, secretion of IL-12 from Syntenin-1-polarized RA CD14⁺CD86⁺GLUT1⁺MΦs, rewires Tbx21⁺Th1 cells via mTOR activation. Further, Syntenin-1-induced arthritogenicity in vivo was dependent on F4/80⁺iNOS*MΦs and their cross-regulation of joint Th1 cells through IL-12 and IL-18 induction. Substantiating these findings, SDC-1^{-/-} mice dysregulated Syntenin-1-mediated arthritis by rebalancing oxidative metabolites AMPK and PPARγ over glycolytic intermediates, GLUT1, HIF1α, and mTOR as well as counteracting a wide range of joint monokines and their inflammatory amplifier, IFNγ. In sum, the results disclosed herein show described and characterize the endogenous ligand, Syntenin-1, its cellular source and mechanism of function in RA. The findings disclosed herein provide evidence that the Syntenin-1/SDC-1 pathway plays an important role in the inflammatory and metabolic landscape of RA through MΦ and T effector cell crosstalk. Since the expression of Syntenin-1/SDC-1 is unaffected by biotherapies in RA circulating cells, this pathway provides a treatment target for patients with RA that are nonresponsive to currently available therapies (e.g., methotrexate (Rheumatrex®, Trexall®), hydroxychloroquine (Plaquenil®), sulfasalazine (Azulfidine®), leflunomide (Arava®), tumor necrosis factor inhibitors (e.g., etanercept (Enbrel®, adalimumab (Humira®)), infliximab (Remicade®), T-cell costimulatory blocking agents (e.g., abatacept (Orencia®), B cell depleting Agents (e.g., rituximab (Rituxan®), jak stat inhibitors, and IL-6 receptors inhibitors.

### Methods of Treatment

Disclosed herein are methods of treating or preventing rheumatoid arthritis in a subject. In some aspects, the methods can comprise administering to the subject a therapeutically effective amount of a syndecan-1 inhibitor. For example, disclosed herein are methods of treating or preventing rheumatoid arthritis in a subject, the method comprising administering to the subject a therapeutically effective amount of a syndecan-1 inhibitor. In some aspects, the syndecan-1 inhibitor can be administered to a subject in need thereof. In some aspects, the subject can be identified as being in need of treatment before the administration step. In some aspects, the subject has rheumatoid arthritis. In some aspects, the subject can be obese.

Disclosed herein are methods of reducing syntenin-1 in synovial fluid or blood in a subject. In some aspects, the methods can comprise administering to the subject a therapeutically effective amount of a syndecan-1 inhibitor. For example, disclosed herein are methods of reducing syntenin-1 in synovial fluid or blood in a subject, the method comprising administering to the subject a therapeutically effective amount of a syndecan-1 inhibitor. In some aspects, the syndecan-1 inhibitor can be administered to a subject in need thereof. In some aspects, the subject can be identified as being in need of treatment before the administration step. In some aspects, the subject has rheumatoid arthritis. In some aspects, the subject has juvenile idiopathic arthritis. In some aspects, the subject can be obese. In some aspects, the subject can be a juvenile. In some aspects, the subject can be 2 years of age or older.

Disclosed herein are methods of reducing chemokine (C—C motif) ligand 2 (CCL2) levels in a subject, the method comprising administering to the subject a therapeutically effective amount of a syndecan-1 inhibitor. In some aspects, the methods disclosed herein can result in the reprogramming or remodeling of the inflammatory or metabolic response of macrophages and their ability to activate Th1 cells. In some aspects, reprogramming or remodeling of the inflammatory or metabolic response of macrophages and their ability to activate Th1 cells can be determined by measuring or determining chemokine (C—C motif) ligand 2 (CCL2) levels. In some aspects, the methods can comprise administering to the subject a therapeutically effective amount of a syndecan-1 inhibitor. In some aspects, the syndecan-1 inhibitor can be administered to a subject in need thereof. In some aspects, the subject can be identified as being in need of treatment before the administration step. In some aspects, the subject has rheumatoid arthritis. In some aspects, the subject has juvenile idiopathic arthritis. In some aspects, the subject can be obese. In some aspects, the subject can be a juvenile. In some aspects, the subject can be 2 years of age or older.

Disclosed herein are methods of reducing cartilage degradation in a subject. In some aspects, the methods can comprise administering to the subject a therapeutically effective amount of a syndecan-1 inhibitor. For example, disclosed herein are methods of reducing cartilage degradation in a subject, the method comprising administering to the subject a therapeutically effective amount of a syndecan-1 inhibitor. In some aspects, the syndecan-1 inhibitor can be administered to a subject in need thereof. In some aspects, the subject can be identified as being in need of treatment before the administration step. In some aspects, the subject has rheumatoid arthritis. In some aspects, the subject has juvenile idiopathic arthritis. In some aspects, the subject can be obese. In some aspects, the subject can be a juvenile. In some aspects, the subject can be 2 years of age or older.

Disclosed herein are methods of reducing synovial inflammation in a subject. In some aspects, the methods can comprise administering to the subject a therapeutically effective amount of a syndecan-1 inhibitor. For example, disclosed herein are methods of reducing synovial inflammation in a subject, the method comprising administering to the subject a therapeutically effective amount of a syndecan-1 inhibitor. In some aspects, the syndecan-1 inhibitor can be administered to a subject in need thereof. In some aspects, the subject can be identified as being in need of treatment before the administration step. In some aspects, the subject has rheumatoid arthritis. In some aspects, the subject has juvenile idiopathic arthritis. In some aspects, the subject can be obese. In some aspects, the subject can be a juvenile. In some aspects, the subject can be 2 years of age or older.

Disclosed herein are methods of reducing or ameliorating one or more symptoms of rheumatoid arthritis in a subject. In some aspects, the methods can comprise administering to the subject a therapeutically effective amount of a syndecan-1 inhibitor. For example, disclosed herein are methods of reducing or ameliorating one or more symptoms of rheumatoid arthritis in a subject, the method comprising administering to the subject a therapeutically effective amount of a syndecan-1 inhibitor. In some aspects, the one or more symptoms of rheumatoid arthritis can be pain, joint tenderness, joint swelling, grip strength, morning stiffness or a combination thereof. In some aspects, the syndecan-1 inhibitor can be administered to a subject in need thereof. In some aspects, the subject can be identified as being in need of treatment before the administration step. In some aspects, the subject has rheumatoid arthritis. In some aspects, the subject can be obese.

Disclosed herein are methods of reducing one or more inflammatory interferon transcription factors in a subject. In some aspects, the methods can comprise administering to the subject a therapeutically effective amount of a syndecan-1 inhibitor. For example, disclosed herein are methods of reducing one or more inflammatory interferon transcription factors in a subject, the method comprising administering to the subject a therapeutically effective amount of a syndecan-1 inhibitor. In some aspects, the one or more inflammatory interferon transcription factors can be IRF1, IRF7, IRF8, IRF9, or a combination thereof. In some aspects, the syndecan-1 inhibitor can be administered to a subject in need thereof. In some aspects, the subject can be identified as being in need of treatment before the administration step. In some aspects, the subject has rheumatoid arthritis. In some aspects, the subject has juvenile idiopathic arthritis. In some aspects, the subject can be obese. In some aspects, the subject can be a juvenile. In some aspects, the subject can be 2 years of age or older.

Disclosed herein are methods of reducing one or more monokines in a subject. In some aspects, the methods can comprise administering to the subject a therapeutically effective amount of a syndecan-1 inhibitor. For example, disclosed herein are methods of reducing one or more monokines in a subject, the method comprising administering to the subject a therapeutically effective amount of a syndecan-1 inhibitor. In some aspects, the one or more monokines can be IL-1β, TNF-α, IL-6, IL-8, CCL2, or a combination thereof. In some aspects, the syndecan-1 inhibitor can be administered to a subject in need thereof. In some aspects, the subject can be identified as being in need of treatment before the administration step. In some aspects, the subject has rheumatoid arthritis. In some aspects, the subject has juvenile idiopathic arthritis. In some aspects, the subject can be obese. In some aspects, the subject can be a juvenile. In some aspects, the subject can be 2 years of age or older.

Disclosed herein are methods of reducing expression of one or more glycolytic factors in a subject. In some aspects, the methods can comprise administering to the subject a therapeutically effective amount of a syndecan-1 inhibitor. For example, disclosed herein are methods of reducing expression of one or more glycolytic factors in a subject, the method comprising administering to the subject a therapeutically effective amount of a syndecan-1 inhibitor. In some aspects, the one or more glycolytic factors can be GLUT1. HK2. mTOR, LDHA or a combination thereof. In some aspects, the syndecan-1 inhibitor can be administered to a subject in need thereof. In some aspects, the subject can be identified as being in need of treatment before the administration step. In some aspects, the subject has rheumatoid arthritis. In some aspects, the subject has juvenile idiopathic arthritis. In some aspects, the subject can be obese. In some aspects, the subject can be a juvenile. In some aspects, the subject can be 2 years of age or older.

Disclosed herein are methods of increasing expression of one or more oxidative intermediates or enzymes in a subject. In some aspects, the methods can comprise administering to the subject a therapeutically effective amount of a syndecan-1 inhibitor. For example, disclosed herein are methods of increasing expression of one or more oxidative intermediates or enzymes in a subject, the method comprising administering to the subject a therapeutically effective amount of a syndecan-1 inhibitor. In some aspects, the one or more oxidative intermediates can be AMPK. In some aspects, the enzyme can be aconitase (ACO2). oxoglutarate dehydrogenase (OGDH). succinate dehydrogenase (SDH2), fumarate hydratase (FH), malate dehydrogenase (MDH), or a combination thereof. In some aspects, the syndecan-1 inhibitor can be administered to a subject in need thereof. In some aspects, the subject can be identified as being in need of treatment before the administration step. In some aspects, the subject has rheumatoid arthritis. In some aspects, the subject has juvenile idiopathic arthritis. In some aspects, the subject can be obese. In some aspects, the subject can be a juvenile. In some aspects, the subject can be 2 years of age or older.

Disclosed herein are methods of treating or preventing juvenile idiopathic arthritis in a subject. In some aspects, the methods can comprise administering to the subject a therapeutically effective amount of a syndecan-1 inhibitor. For example, disclosed herein are methods of treating or preventing juvenile idiopathic arthritis in a subject, the method comprising administering to the subject a therapeutically effective amount of a syndecan-1 inhibitor. In some aspects, the syndecan-1 inhibitor can be administered to a subject in need thereof. In some aspects, the subject can be identified as being in need of treatment before the administration step. In some aspects, the subject has juvenile idiopathic arthritis. In some aspects, the subject can be a juvenile. In some aspects, the subject can be 2 years of age or older.

Disclosed herein are methods of treating or preventing psoriatic arthritis in a subject. In some aspects, the methods can comprise administering to the subject a therapeutically effective amount of a syndecan-1 inhibitor. For example, disclosed herein are methods of treating or preventing psoriatic arthritis in a subject, the method comprising administering to the subject a therapeutically effective amount of a syndecan-1 inhibitor. In some aspects, the syndecan-1 inhibitor can be administered to a subject in need thereof. In some aspects, the subject can be identified as being in need of treatment before the administration step. In some aspects, the subject has psoriatic arthritis. In some aspects, the subject can be obese.

Disclosed herein are methods of treating or preventing ankylosing spondylitis in a subject. In some aspects, the methods can comprise administering to the subject a therapeutically effective amount of a syndecan-1 inhibitor. For example, disclosed herein are methods of treating or preventing ankylosing spondylitis in a subject, the method comprising administering to the subject a therapeutically effective amount of a syndecan-1 inhibitor. In some aspects, the syndecan-1 inhibitor can be administered to a subject in need thereof. In some aspects, the subject can be identified as being in need of treatment before the administration step. In some aspects, the subject has ankylosing spondylitis. In some aspects, the subject can be obese.

Disclosed herein are methods of treating or preventing Crohn's disease in a subject. In some aspects, the methods can comprise administering to the subject a therapeutically effective amount of a syndecan-1 inhibitor. For example, disclosed herein are methods of treating or preventing Crohn's disease in a subject, the method comprising administering to the subject a therapeutically effective amount of a syndecan-1 inhibitor. In some aspects, the syndecan-1 inhibitor can be administered to a subject in need thereof. In some aspects, the subject can be identified as being in need of treatment before the administration step. In some aspects, the subject has Crohn's disease. In some aspects, the subject can be obese. In some aspects, the subject can be a pediatric patient. In some aspects, the subject can be an adult patient. In some aspects, the subject can be 6 years of age or older.

Disclosed herein are methods treating or preventing ulcerative colitis in a subject. In some aspects, the methods can comprise administering to the subject, a therapeutically effective amount of a syndecan-1 inhibitor. For example, disclosed herein are methods of treating or preventing ulcerative colitis in a subject, the method comprising administering to the subject a therapeutically effective amount of a syndecan-1 inhibitor. In some aspects, the syndecan-1 inhibitor can be administered to a subject in need thereof. In some aspects, the subject can be identified as being in need of treatment before the administration step. In some aspects, the subject has ulcerative colitis. In some aspects, the subject can be obese. In some aspects, the subject can be a pediatric patient. In some aspects, the subject can be an adult patient. In some aspects, the subject can be 5 years of age or older.

Disclosed herein are methods treating or preventing plaque psoriasis in a subject. In some aspects, the methods can comprise administering to the subject, a therapeutically effective amount of a syndecan-1 inhibitor. For example, disclosed herein are methods of treating or preventing plaque psoriasis in a subject, the methods comprising administering to the subject a therapeutically effective amount of a syndecan-1 inhibitor. In some aspects, the syndecan-1 inhibitor can be administered to a subject in need thereof. In some aspects, the subject can be identified as being in need of treatment before the administration step. In some aspects, the subject has plaque psoriasis. In some aspects, the subject can be obese.

Disclosed herein are methods treating or preventing hidradenitis suppurativa in a subject. In some aspects, the methods can comprise administering to the subject, a therapeutically effective amount of a syndecan-1 inhibitor. For example, disclosed herein are methods of treating or preventing hidradenitis suppurativa in a subject, the method comprising administering to the subject a therapeutically effective amount of a syndecan-1 inhibitor. In some aspects, the syndecan-1 inhibitor can be administered to a subject in need thereof. In some aspects, the subject can be identified as being in need of treatment before the administration step. In some aspects, the subject has hidradenitis suppurativa. In some aspects, the subject can be obese. In some aspects, the subject can be 12 years of age or older.

Disclosed herein are methods treating or preventing uveitis in a subject. In some aspects, the methods can comprise administering to the subject, a therapeutically effective amount of a syndecan-1 inhibitor. For example, disclosed herein are methods of treating or preventing uveitis in a subject, the method comprising administering to the subject a therapeutically effective amount of a syndecan-1 inhibitor. In some aspects, the syndecan-1 inhibitor can be administered to a subject in need thereof. In some aspects, the subject can be identified as being in need of treatment before the administration step. In some aspects, the subject has uveitis. In some aspects, the subject can be obese. In some aspects, the subject can be a pediatric patient. In some aspects, the subject can be an adult patient. In some aspects, the subject can be 2 years of age or older. In some aspects, the uveitis can be non-infectious, intermediate, posterior, or panuveitis.

Disclosed herein are methods of reducing or ameliorating one or more symptoms of idiopathic juvenile arthritis in a subject. In some aspects, the methods can comprise administering to the subject a therapeutically effective amount of a syndecan-1 inhibitor. For example, disclosed herein are methods of reducing or ameliorating one or more symptoms of idiopathic juvenile arthritis in a subject, the method comprising administering to the subject a therapeutically effective amount of a syndecan-1 inhibitor. In some aspects, the one or more symptoms of idiopathic juvenile arthritis can be pain, joint tenderness. joint swelling, grip strength, morning stiffness, eye inflammation, fatigue, decreased appetite, poor weight gain, slow growth, high fever, rash, swollen lymph nodes, or a combination thereof. In some aspects, the syndecan-1 inhibitor can be administered to a subject in need thereof. In some aspects, the subject can be identified as being in need of treatment before the administration step. In some aspects, the subject has idiopathic juvenile arthritis. In some aspects, the subject can be obese. In some aspects, the subject can be a juvenile. In some aspects, the subject can be 2 years of age or older. In some aspects, the juvenile idiopathic arthritis can be active polyarticular juvenile idiopathic arthritis.

Disclosed herein are methods of reducing or ameliorating one or more symptoms of psoriatic arthritis in a subject. In some aspects, the methods can comprise administering to the subject a therapeutically effective amount of a syndecan-1 inhibitor. For example, disclosed herein are methods of reducing or ameliorating one or more symptoms of psoriatic arthritis in a subject, the method comprising administering to the subject a therapeutically effective amount of a syndecan-1 inhibitor. In some aspects, the one or more symptoms of psoriatic arthritis can be pain, joint tenderness, joint swelling morning stiffness, itching, tendinopathy, skin or rash, or a combination thereof. In some aspects, the syndecan-1 inhibitor can be administered to a subject in need thereof. In some aspects, the subject can be identified as being in need of treatment before the administration step. In some aspects, the subject has psoriatic arthritis. In some aspects, the subject can be obese.

Disclosed herein are methods of reducing or ameliorating one or more symptoms of ankylosing spondylitis in subject. In some aspects, the methods can comprise administering to the subject a therapeutically effective amount of a syndecan-1 inhibitor. For example, disclosed herein are methods of reducing or ameliorating one or more symptoms of ankylosing spondylitis in a subject, the method comprising administering to the subject a therapeutically effective amount of a syndecan-1 inhibitor. In some aspects, the one or more symptoms of ankylosing spondylitis can be pain, joint tenderness, morning stiffness, stooped posture, appetite loss, weight loss, fatigue, fever, anemia, eye inflammation, blurred vision or sensitivity to light, back joint dysfunction, inflammatory bowel disease, or a combination thereof. In some aspects, the syndecan-1 inhibitor can be administered to a subject in need thereof. In some aspects, the subject can be identified as being in need of treatment before the administration step. In some aspects, the subject has ankylosing spondylitis. In some aspects, the subject can be obese.

In some aspects, the syndecan-1 inhibitor administered to a subject in the methods disclosed herein can be a mouse anti-SDC1 monoclonal antibody. In some aspects, the mouse anti-SDC1 monoclonal antibody BA38. In some aspects, the syndecan-1 inhibitor can be BT062-DM4 (indatuximab Ravtansine). In some aspects, the syndecan-1 inhibitor B-134. In some aspects, the syndecan-1 inhibitor can be VIS832. In some aspects, the syndecan-1 inhibitor can be 4B3. In some aspects, the syndecan-1 inhibitor can be OC-46F2. In some aspects, the syndecan-1 inhibitor can be ULBP2-BB4. In some aspects, the syndecan-1 inhibitor can be CART-138. In some aspects, the syndecan-1 inhibitor can be CD138.CAR. In some aspects, the syndecan-1 inhibitor can be CD138-specific CAR-NK. In some aspects, the syndecan-1 inhibitor can be GLVGLIFAV (SEQ ID NO: 1: PVX-410). In some aspects, the syndecan-1 inhibitor can by synstatin.

In some aspects, the syndecan-1 inhibitor prevents, inhibits, or reduces syndecan-1 from binding to the PDZ-2 domain of syntenin-1.

In some aspects of the methods disclosed herein, T effector cell differentiation into Th1 or Th17 can be modulated or prevented in the subject.

Disclosed herein are methods of treating or preventing rheumatoid arthritis in a subject. In some aspects, the methods can comprise administering to the subject a therapeutically effective amount of a syntenin-1 inhibitor. For example, disclosed herein are methods of treating or preventing rheumatoid arthritis in a subject, the method comprising administering to the subject a therapeutically effective amount of a syntenin-1 inhibitor. In some aspects, the syntenin-1 inhibitor can be administered to a subject in need thereof. In some aspects, the subject can be identified as being in need of treatment before the administration step. In some aspects, the subject has rheumatoid arthritis. In some aspects, the subject can be obese.

Disclosed herein are methods of reducing syntenin-1 in synovial fluid or blood in a subject. In some aspects, the methods can comprise administering to the subject a therapeutically effective amount of a syntenin-1 inhibitor. For example, disclosed herein are methods of reducing syntenin-1 in synovial fluid or blood in a subject, the method comprising administering to the subject a therapeutically effective amount of a syntenin-1 inhibitor. In some aspects, the syntenin-1 inhibitor can be administered to a subject in need thereof. In some aspects, the subject can be identified as being in need of treatment before the administration step. In some aspects, the subject has rheumatoid arthritis. In some aspects, the subject has juvenile idiopathic arthritis. In some aspects, the subject can be obese. In some aspects, the subject can be a juvenile. In some aspects, the subject can be 2 years of age or older.

Disclosed herein are methods of reducing chemokine (C—C motif) ligand 2 (CCL2) levels in a subject. In some aspects, the methods disclosed herein can result in modifying macrophage levels which can result in the reprogramming or remodeling of the inflammatory or metabolic response of macrophages and their ability to activate Th1 cells. In some aspects, reprogramming or remodeling of the inflammatory or metabolic response of macrophages and their ability to activate Th1 cells can be determined by measuring or determining chemokine (C—C motif) ligand 2 (CCL2) levels. In some aspects, the methods can comprise administering to the subject a therapeutically effective amount of a syntenin-1 inhibitor. For example. disclosed herein are methods of reducing syntenin-1-instigated RA macrophage reprogramming in a subject, the method comprising administering to the subject a therapeutically effective amount of a syntenin-1 inhibitor. In some aspects, the syntenin-1 inhibitor can be administered to a subject in need thereof. In some aspects, the subject can be identified as being in need of treatment before the administration step. In some aspects, the subject has rheumatoid arthritis. In some aspects, the subject has juvenile idiopathic arthritis. In some aspects, the subject can be obese. In some aspects, the subject can be a juvenile. In some aspects, the subject can be 2 years of age or older.

Disclosed herein are methods of reducing cartilage degradation in a subject. In some aspects, the methods can comprise administering to the subject a therapeutically effective amount of a syntenin-1 inhibitor. For example, disclosed herein are methods of reducing cartilage degradation in a subject, the method comprising administering to the subject a therapeutically effective amount of a syntenin-1 inhibitor. In some aspects, the syntenin-1 inhibitor can be administered to a subject in need thereof. In some aspects, the subject can be identified as being in need of treatment before the administration step. In some aspects, the subject has rheumatoid arthritis. In some aspects, the subject has juvenile idiopathic arthritis. In some aspects, the subject can be obese. In some aspects, the subject can be a juvenile. In some aspects, the subject can be 2 years of age or older.

Disclosed herein are methods of reducing synovial inflammation in a subject. In some aspects, the methods can comprise administering to the subject a therapeutically effective amount of a syntenin-1 inhibitor. For example, disclosed herein are methods of reducing synovial inflammation in a subject, the method comprising administering to the subject a therapeutically effective amount of a syntenin-1 inhibitor. In some aspects, the syntenin-1 inhibitor can be administered to a subject in need thereof. In some aspects, the subject can be identified as being in need of treatment before the administration step. In some aspects, the subject has rheumatoid arthritis. In some aspects, the subject has juvenile idiopathic arthritis. In some aspects, the subject can be obese. In some aspects, the subject can be a juvenile. In some aspects, the subject can be 2 years of age or older.

Disclosed herein are methods of reducing or ameliorating one or more symptoms of rheumatoid arthritis in a subject. In some aspects, the methods can comprise administering to the subject a therapeutically effective amount of a syntenin-1 inhibitor. For example, disclosed herein are methods of reducing or ameliorating one or more symptoms of rheumatoid arthritis in a subject, the method comprising administering to the subject a therapeutically effective amount of a syntenin-1 inhibitor. In some aspects, the one or more symptoms of rheumatoid arthritis can be pain, joint tenderness, joint swelling, grip strength, morning stiffness or a combination thereof. In some aspects, the syntenin-1 inhibitor can be administered to a subject in need thereof. In some aspects, the subject can be identified as being in need of treatment before the administration step. In some aspects, the subject has rheumatoid arthritis. In some aspects, the subject can be obese.

Disclosed herein are methods of reducing one or more inflammatory interferon transcription factors in a subject. In some aspects, the methods can comprise administering to the subject a therapeutically effective amount of a syntenin-1 inhibitor. For example, disclosed herein are methods of reducing one or more inflammatory interferon transcription factors in a subject, the method comprising administering to the subject a therapeutically effective amount of a syntenin-1 inhibitor. In some aspects, the one or more inflammatory interferon transcription factors can be IRF1. IRF7, IRF8, IRF9, or a combination thereof. In some aspects, the syntenin-1 inhibitor can be administered to a subject in need thereof. In some aspects, the subject can be identified as being in need of treatment before the administration step. In some aspects, the subject has rheumatoid arthritis. In some aspects, the subject can be obese.

Disclosed herein are methods of reducing one or more monokines in a subject. In some aspects, the methods can comprise administering to the subject a therapeutically effective amount of a syntenin-1 inhibitor. For example, disclosed herein are methods of reducing one or more monokines in a subject, the method comprising administering to the subject a therapeutically effective amount of a syntenin-1 inhibitor. In some aspects, the one or more monokines can be IL-1β, TNF-α, IL-6, IL-8, CCL2, or a combination thereof. In some aspects, the syntenin-1 inhibitor can be administered to a subject in need thereof. In some aspects, the subject can be identified as being in need of treatment before the administration step. In some aspects, the subject has rheumatoid arthritis. In some aspects, the subject can be obese.

Disclosed herein are methods of reducing expression of one or more glycolytic factors in a subject. In some aspects, the methods can comprise administering to the subject a therapeutically effective amount of a syntenin-1 inhibitor. For example, disclosed herein are methods of reducing expression of one or more glycolytic factors in a subject, the method comprising administering to the subject a therapeutically effective amount of a syntenin-1 inhibitor. In some aspects, the one or more glycolytic factors can be GLUT1, HK2, mTOR, LDHA or a combination thereof. In some aspects, the syntenin-1 inhibitor can be administered to a subject in need thereof. In some aspects, the subject can be identified as being in need of treatment before the administration step. In some aspects, the subject has rheumatoid arthritis. In some aspects, the subject can be obese.

Disclosed herein are methods of increasing expression of one or more oxidative intermediates or enzymes in a subject. In some aspects, the methods can comprise administering to the subject a therapeutically effective amount of a syntenin-1 inhibitor. For example, disclosed herein are methods of increasing expression of one or more oxidative intermediates or enzymes in a subject, the method comprising administering to the subject a therapeutically effective amount of a syntenin-1 inhibitor. In some aspects, the one or more oxidative intermediates can be AMPK. In some aspects, the enzyme can be aconitase (ACO2), oxoglutarate dehydrogenase (OGDH), succinate dehydrogenase (SDH2), fumarate hydratase (FH), malate dehydrogenase (MDH), or a combination thereof. In some aspects, the syntenin-1 inhibitor can be administered to a subject in need thereof. In some aspects, the subject can be identified as being in need of treatment before the administration step. In some aspects, the subject has rheumatoid arthritis. In some aspects, the subject can be obese.

Disclosed herein are methods of treating or preventing juvenile idiopathic arthritis in a subject. In some aspects, the methods can comprise administering to the subject a therapeutically effective amount of a syntenin-1 inhibitor. For example, disclosed herein are methods of treating or preventing juvenile idiopathic arthritis in a subject, the method comprising administering to the subject a therapeutically effective amount of a syntenin-1 inhibitor. In some aspects, the syntenin-1 inhibitor can be administered to a subject in need thereof. In some aspects, the subject can be identified as being in need of treatment before the administration step. In some aspects, the subject has juvenile idiopathic arthritis. In some aspects, the subject can be a juvenile. In some aspects, the subject can be 2 years of age or older.

Disclosed herein are methods of treating or preventing psoriatic arthritis in a subject. In some aspects, the methods can comprise administering to the subject a therapeutically effective amount of a syntenin-1 inhibitor. For example, disclosed herein are methods of treating or preventing psoriatic arthritis in a subject, the method comprising administering to the subject a therapeutically effective amount of a syntenin-1 inhibitor. In some aspects, the syntenin-1 inhibitor can be administered to a subject in need thereof. In some aspects, the subject can be identified as being in need of treatment before the administration step. In some aspects, the subject has psoriatic arthritis. In some aspects, the subject can be obese.

Disclosed herein are methods of treating or preventing ankylosing spondylitis in a subject. In some aspects, the methods can comprise administering to the subject a therapeutically effective amount of a syntenin-1 inhibitor. For example, disclosed herein are methods of treating or preventing ankylosing spondylitis in a subject, the method comprising administering to the subject a therapeutically effective amount of a syntenin-1 inhibitor. In some aspects, the syntenin-1 inhibitor can be administered to a subject in need thereof. In some aspects, the subject can be identified as being in need of treatment before the administration step. In some aspects, the subject has ankylosing spondylitis. In some aspects, the subject can be obese.

Disclosed herein are methods of treating or preventing Crohn's disease in a subject. In some aspects, the methods can comprise administering to the subject a therapeutically effective amount of a syntenin-1 inhibitor. For example, disclosed herein are methods of treating or preventing Crohn's disease in a subject, the method comprising administering to the subject a therapeutically effective amount of a syntenin-1 inhibitor. In some aspects, the syntenin-1 inhibitor can be administered to a subject in need thereof. In some aspects, the subject can be identified as being in need of treatment before the administration step. In some aspects, the subject has Crohn's disease. In some aspects, the subject can be obese. In some aspects, the subject can be a pediatric patient. In some aspects, the subject can be an adult patient. In some aspects, the subject can be 6 years of age or older.

Disclosed herein are methods treating or preventing ulcerative colitis in a subject. In some aspects, the methods can comprise administering to the subject, a therapeutically effective amount of a syntenin-1 inhibitor. For example, disclosed herein are methods of treating or preventing ulcerative colitis in a subject, the method comprising administering to the subject a therapeutically effective amount of a syntenin-1 inhibitor. In some aspects, the syntenin-1 inhibitor can be administered to a subject in need thereof. In some aspects, the subject can be identified as being in need of treatment before the administration step. In some aspects, the subject has ulcerative colitis. In some aspects, the subject can be obese. In some aspects, the subject can be a pediatric patient. In some aspects, the subject can be an adult patient. In some aspects, the subject can be 5 years of age or older.

Disclosed herein are methods treating or preventing plaque psoriasis in a subject. In some aspects, the methods can comprise administering to the subject, a therapeutically effective amount of a syntenin-1 inhibitor. For example, disclosed herein are methods of treating or preventing plaque psoriasis in a subject, the methods comprising administering to the subject a therapeutically effective amount of a syntenin-1 inhibitor. In some aspects, the syntenin-1 inhibitor can be administered to a subject in need thereof. In some aspects, the subject can be identified as being in need of treatment before the administration step. In some aspects, the subject has plaque psoriasis. In some aspects, the subject can be obese.

Disclosed herein are methods treating or preventing hidradenitis suppurativa in a subject. In some aspects, the methods can comprise administering to the subject, a therapeutically effective amount of a syntenin-1 inhibitor. For example, disclosed herein are methods of treating or preventing hidradenitis suppurativa in a subject, the method comprising administering to the subject a therapeutically effective amount of a syntenin-1 inhibitor. In some aspects, the syntenin-1 inhibitor can be administered to a subject in need thereof. In some aspects, the subject can be identified as being in need of treatment before the administration step. In some aspects, the subject has hidradenitis suppurativa. In some aspects, the subject can be obese. In some aspects, the subject can be 12 years of age or older.

Disclosed herein are methods treating or preventing uveitis in a subject. In some aspects, the methods can comprise administering to the subject. a therapeutically effective amount of a syntenin-1 inhibitor. For example, disclosed herein are methods of treating or preventing uveitis in a subject, the method comprising administering to the subject a therapeutically effective amount of a syntenin-1 inhibitor. In some aspects, the syntenin-1 inhibitor can be administered to a subject in need thereof. In some aspects, the subject can be identified as being in need of treatment before the administration step. In some aspects, the subject has uveitis. In some aspects, the subject can be obese. In some aspects, the subject can be a pediatric patient. In some aspects, the subject can be an adult patient. In some aspects, the subject can be 2 years of age or older. In some aspects, the uveitis can be non-infectious, intermediate, posterior, or panuveitis.

Disclosed herein are methods of reducing or ameliorating one or more symptoms of idiopathic juvenile arthritis in a subject. In some aspects, the methods can comprise administering to the subject a therapeutically effective amount of a syntenin-1 inhibitor. For example, disclosed herein are methods of reducing or ameliorating one or more symptoms of idiopathic juvenile arthritis in a subject, the method comprising administering to the subject a therapeutically effective amount of a syntenin-1 inhibitor. In some aspects, the one or more symptoms of idiopathic juvenile arthritis can be pain, joint tenderness, joint swelling, grip strength, morning stiffness, eye inflammation, fatigue, decreased appetite, poor weight gain, slow growth. high fever, rash, swollen lymph nodes, or a combination thereof. In some aspects, the syntenin-1 inhibitor can be administered to a subject in need thereof. In some aspects, the subject can be identified as being in need of treatment before the administration step. In some aspects, the subject has idiopathic juvenile arthritis. In some aspects, the subject can be obese. In some aspects, the subject can be a juvenile. In some aspects, the subject can be 2 years of age or older. In some aspects, the juvenile idiopathic arthritis can be active polyarticular juvenile idiopathic arthritis.

Disclosed herein are methods of reducing or ameliorating one or more symptoms of psoriatic arthritis in a subject. In some aspects, the methods can comprise administering to the subject a therapeutically effective amount of a syntenin-1 inhibitor. For example, disclosed herein are methods of reducing or ameliorating one or more symptoms of psoriatic arthritis in a subject, the method comprising administering to the subject a therapeutically effective amount of a syntenin-1 inhibitor. In some aspects, the one or more symptoms of psoriatic arthritis can be pain, joint tenderness, joint swelling morning stiffness, itching, tendinopathy, skin or rash, or a combination thereof. In some aspects, the syntenin-1 inhibitor can be administered to a subject in need thereof. In some aspects, the subject can be identified as being in need of treatment before the administration step. In some aspects, the subject has psoriatic arthritis. In some aspects, the subject can be obese.

Disclosed herein are methods of reducing or ameliorating one or more symptoms of ankylosing spondylitis in subject. In some aspects, the methods can comprise administering to the subject a therapeutically effective amount of a syntenin-1 inhibitor. For example, disclosed herein are methods of reducing or ameliorating one or more symptoms of ankylosing spondylitis in a subject, the method comprising administering to the subject a therapeutically effective amount of a syntenin-1 inhibitor. In some aspects, the one or more symptoms of ankylosing spondylitis can be pain, joint tenderness, morning stiffness, stooped posture, appetite loss, weight loss, fatigue. fever, anemia, eye inflammation, blurred vision or sensitivity to light, back joint dysfunction, inflammatory bowel disease, or a combination thereof. In some aspects, the syntenin-1 inhibitor can be administered to a subject in need thereof. In some aspects, the subject can be identified as being in need of treatment before the administration step. In some aspects, the subject has ankylosing spondylitis. In some aspects, the subject can be obese.

In some aspects, the syndecan-1 inhibitor can be a peptide that binds to syndecan-1 or a peptide that is capable of binding to syndecan-1 and blocks signaling or activity of syndecan-1. In some aspects, the syntenin-1 inhibitor can be a peptide that binds to syntenin-1 or a peptide that is capable of binding to syntenin-1 and blocks signaling or activity of syntenin-1. In some aspects, the disclosed inhibitors can be a peptide disclosed in Tao et al., Mol. Ther. 2008, 16(11): 1776-1782, which is incorporated by reference herein for its teaching of inhibitors.

In some aspects, the syntenin-1 inhibitor prevents, inhibits, or reduces syndecan-1 from binding to the PDZ-2 domain of syntenin-1.

In some aspects, the syntenin-1 inhibitor binds to the PDZ-2 domain of syntenin-1 thereby preventing syndecan-1 from binding to the PDZ-2 domain of syntenin-1.

The compositions described herein can be formulated to include a therapeutically effective amount of a syndecan-1 inhibitor or a syntenin-1 inhibitor described herein. Therapeutic administration encompasses prophylactic applications (e.g., or preventing rheumatoid arthritis). Based on genetic testing and other prognostic methods, a physician in consultation with their patient can choose a prophylactic administration where the patient has a clinically determined predisposition or increased susceptibility (in some cases, a greatly increased susceptibility) to rheumatoid arthritis.

The compositions described herein can be administered to the subject (e.g., a human patient) in an amount sufficient to delay, reduce, or preferably prevent the onset of clinical disease. Accordingly, in some aspects, the patient can be a human patient. In therapeutic applications, compositions can be administered to a subject (e.g., a human patient) already with or diagnosed with rheumatoid arthritis, increased levels or amounts of syntenin-1 or syndecan-1 in synovial fluid or blood of a subject. cartilage degradation, synovial inflammation, or one or more symptoms of rheumatoid arthritis in an amount sufficient to at least partially improve a sign or symptom or to inhibit the progression of (and preferably arrest) the symptoms of the condition, its complications, and consequences. An amount adequate to accomplish this is defined as a “therapeutically effective amount.” A therapeutically effective amount of a composition (e.g., a pharmaceutical composition) can be an amount that achieves a cure, but that outcome is only one among several that can be achieved. As noted, a therapeutically effective amount includes amounts that provide a treatment in which the onset or progression of the disease, disorder, condition or injury is delayed, hindered, or prevented, or the disease, disorder, condition or injury or a symptom of the disease, disorder, condition or injury is ameliorated or its frequency can be reduced. One or more of the symptoms can be less severe. Recovery can be accelerated in an individual who has been treated. For example, treatment of rheumatoid arthritis may involve, for example, a reduction in inflammation, a reduction in cartilage degradation, reprogramming or remodeling of the inflammatory or metabolic response of macrophages, a reduction of one or more inflammatory interferon transcription factors, a reduction of one or more monokines, a reduction in the expression of one or more glycolytic factors, an increase in the expression of one or more oxidative intermediates, or a reduction or prevention of pain.

In some aspects, the syndecan-1 or syntenin-1 inhibitor can be administered with at least a second therapeutic agent. The methods and compositions, including combination therapies, can enhance the therapeutic or protective effect, and/or increase the therapeutic effect to any of the syntenin-1 or syndecan-1 inhibitors described herein.

The syntenin-1 or syndecan-1 inhibitors can be administered before, during, after, or in various combinations relative to a second therapeutic agent or therapy. The administrations may be in intervals ranging from concurrently to minutes to days to weeks. In aspects where the syntenin-1 or syndecan-1 inhibitors is provided to a patient separately from a second therapeutic agent or therapy, one would generally ensure that a significant period of time did not expire between the time of each delivery, such that the two compounds would still be able to exert an advantageously combined effect on the patient. In such instances, it is contemplated that one may provide a patient with the syntenin-1 inhibitor or the syndecan-1 inhibitor and the second therapeutic agent or therapy within about 12 to 24 or 72 h of each other and, more particularly, within about 6-12 h of each other. In some situations, it may be desirable to extend the time period for treatment significantly where several days (2, 3, 4, 5, 6, or 7) to several weeks (1, 2, 3, 4, 5, 6, 7, or 8) lapse between respective administrations.

In some aspects, a course of treatment can last between 1-90 days or more (this such range includes intervening days). It is contemplated that one agent may be given on any day of day 1 to day 90 (this such range includes intervening days) or any combination thereof, and another agent is given on any day of day 1 to day 90 (this such range includes intervening days) or any combination thereof. Within a single day (24-hour period), the patient may be given one or multiple administrations of the agent(s). Moreover, after a course of treatment, it is contemplated that there can be a period of time at which no anti-cancer treatment is administered. This time period may last 1-7 days, and/or 1-5 weeks, and/or 1-12 months or more (this such range includes intervening days), depending on the condition of the patient, such as their prognosis, strength. health. etc. It is expected that the treatment cycles would be repeated as necessary.

Various combinations may be employed. For the example below a syntenin-1 inhibitor or a syndecan-1 inhibitor is “A” and a second therapeutic agent is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A B/A/B/A
B/A/A/B A/A/A/B B/A/A/A A/B/A/A A/A/B/A.

Administration of any compound or therapy disclosed herein to a patient will follow general protocols for the administration of such compounds. taking into account the toxicity, if any, of the agents. Therefore, in some aspects there can be a step of monitoring toxicity that can be attributable to combination therapy.

In some aspects, the second therapeutic agent can be a nonsteroidal anti-inflammatory drug. In some aspects, the second therapeutic agent can be a disease modifying anti-rheumatic drug. In some aspects, the disease modifying anti-rheumatic drug can be methotrexate (Rheumatrext, Trexall®), hydroxychloroquine (Plaquenil®), sulfasalazine (Azulfidine®), leflunomide (AravaC®), tumor necrosis factor inhibitors (e.g., etanercept (Enbrel®), adalimumab (Humira®), infliximab (Remicade®), certolizumab pegol (Cimzia®), and golimumab (Simponi®)), T-cell costimulatory blocking agents (e.g., abatacept (Orencia®), B cell depleting agents (e.g., rituximab (Rituxan®), interleukin-6 inhibitors (e.g., tocilizumab (ActemraR), interleukin-1 receptor antagonists (e.g., anakinra (Kineret®), intramuscular gold, and other immunomodulatorv and cytoxic agents (e.g., azathioprine (Imuran®) and cvclosporine A (Neoral®, Sandimmune®). In some aspects, the second therapeutic agent can be a therapy. For example, the second therapeutic agent or therapy can be a joint replacement surgery.

The compositions described herein used in the disclosed methods can be formulated to include a therapeutically effective amount of the syndecan-1 inhibitor or the syntenin-1 inhibitor disclosed herein. In some aspects, the syndecan-1 inhibitor or the syntenin-1 inhibitor thereof disclosed herein can be contained within a pharmaceutical formulation. In some aspects, the pharmaceutical formulation can be a unit dosage formulation.

The therapeutically effective amount or dosage of any of the syndecan-1 inhibitors or the syntenin-1 inhibitors used in the methods as disclosed herein applied to mammals (e.g., humans) can be determined by one of ordinary skill in the art with consideration of individual differences in age, weight, sex, the severity of the subject's symptoms, and the particular composition or route of administration selected, other drugs administered and the judgment of the attending clinician. Variations in the needed dosage may be expected. Variations in dosage levels can be adjusted using standard empirical routes for optimization. The particular dosage of a pharmaceutical composition to be administered to the patient will depend on a variety of considerations (e.g., the severity of the symptoms), the age and physical characteristics of the subject and other considerations known to those of ordinary skill in the art. Dosages can be established using clinical approaches known to one of ordinary skill in the art. A therapeutically effective dosage of the syndecan-1 inhibitor or the syntenin-1 inhibitor can result in a decrease in severity of one or more disease symptoms, an increase in frequency and duration of disease symptom-free periods, or a prevention of impairment or disability due to the disease affliction. As disclosed therein, in some aspects a therapeutically effective amount of a syndecan-1 inhibitor or a syntenin-1 inhibitor can reduce syndecan-1 or syntenin-1 in synovial fluid, modify macrophage levels, decrease cartilage degradation, decrease synovial inflammation, reduce one or more inflammatory interferon transcription factors, reduce one or more monokines, reduce expression of one or more glycolytic factors, increase expression of one or more oxidative intermediates or enzymes, or otherwise reduce or ameliorate one or more symptoms in a subject.

The duration of treatment with any composition in the methods disclosed herein can be any length of time from as short as one day to as long as the life span of the host (e.g., many years). For example, the compositions can be administered once a week (for, for example, 4 weeks to many months or years); once a month (for, for example, three to twelve months or for many years); or once a year for a period of 5 years, ten years, or longer. It is also noted that the frequency of treatment can be variable. For example, the present compositions can be administered once (or twice, three times, etc.) daily, weekly, monthly, or yearly.

The total effective amount of the syndecan-1 inhibitor or the syntenin-1 inhibitor as disclosed herein can be administered to a subject as a single dose. either as a bolus or by infusion over a relatively short period of time, or can be administered using a fractionated treatment protocol in which multiple doses are administered over a more prolonged period of time. Alternatively, continuous intravenous infusions sufficient to maintain therapeutically effective concentrations in the blood are also within the scope of the present disclosure.

### Pharmaceutical Compositions

As disclosed herein, are pharmaceutical compositions, comprising one or more of the therapeutic compositions or syndecan-1 inhibitors or syntenin-1 inhibitors disclosed herein. As disclosed herein, are pharmaceutical compositions, comprising a syndecan-1 inhibitor or a syntenin-1 inhibitor and a pharmaceutical acceptable carrier described herein. In some aspects, the syndecan-1 inhibitor or the syntenin-1 inhibitor can be formulated for oral or parental administration. In some aspects, the parental administration can be intravenous, subcutaneous, intramuscular or direct injection. In some aspects, the syndecan-1 inhibitor or the syntenin-1 inhibitor can be administered intramuscularly, intravenously, subcutaneously, orally, topically, transdermally, sublingually, or intra-articularly. The compositions can be formulated for administration by any of a variety of routes of administration, and can include one or more physiologically acceptable excipients, which can vary depending on the route of administration. As used herein, the term “excipient” means any compound or substance, including those that can also be referred to as “carriers” or “diluents.” Preparing pharmaceutical and physiologically acceptable compositions is considered routine in the art, and thus, one of ordinary skill in the art can consult numerous authorities for guidance if needed.

The compositions can be administered directly to a subject. Generally, the compositions can be suspended in a pharmaceutically acceptable carrier (e.g., physiological saline or a buffered saline solution) to facilitate their delivery. Encapsulation of the compositions in a suitable delivery vehicle (e.g., polymeric microparticles or implantable devices) may increase the efficiency of delivery.

The compositions can be formulated in various ways for parenteral or nonparenteral administration. Where suitable, oral formulations can take the form of tablets, pills, capsules, or powders, which may be enterically coated or otherwise protected. Sustained release formulations, suspensions, elixirs, aerosols, and the like can also be used.

Pharmaceutically acceptable carriers and excipients can be incorporated (e.g., water, saline, aqueous dextrose, and glycols, oils (including those of petroleum, animal, vegetable or synthetic origin), starch, cellulose, talc, glucose, lactose, sucrose, gelatin, malt, rice, flour, chalk, silica gel, magnesium stearate, sodium stearate, glycerol monosterate, sodium chloride, dried skim milk, glycerol, propylene glycol, ethanol, and the like). The compositions may be subjected to conventional pharmaceutical expedients such as sterilization and may contain conventional pharmaceutical additives such as preservatives, stabilizing agents, wetting or emulsifying agents, salts for adjusting osmotic pressure, buffers, and the like. Suitable pharmaceutical carriers and their formulations are described in “Remington's Pharmaceutical Sciences” by E. W. Martin, which is herein incorporated by reference. Such compositions will, in any event, contain an effective amount of the compositions together with a suitable amount of carrier so as to prepare the proper dosage form for proper administration to the patient.

The pharmaceutical compositions as disclosed herein can be prepared for oral or parenteral administration. Pharmaceutical compositions prepared for parenteral administration include those prepared for intravenous (or intra-arterial), intramuscular, subcutaneous, intraperitoneal, transmucosal (e.g., intranasal, intravaginal, or rectal), or transdermal (e.g., topical) administration. Aerosol inhalation can also be used. Thus, compositions can be prepared for parenteral administration that includes any of the syndecan-1 inhibitors or the syntenin-1 inhibitors dissolved or suspended in an acceptable carrier, including but not limited to an aqueous carrier, such as water, buffered water, saline, buffered saline (e.g., PBS), and the like. One or more of the excipients included can help approximate physiological conditions, such as pH adjusting and buffering agents, tonicity adjusting agents, wetting agents, detergents, and the like. Where the compositions include a solid component (as they may for oral administration), one or more of the excipients can act as a binder or filler (e.g., for the formulation of a tablet, a capsule, and the like).

The pharmaceutical compositions can be sterile and sterilized by conventional sterilization techniques or sterile filtered. Aqueous solutions can be packaged for use as is, or lyophilized, the lyophilized preparation, which is encompassed by the present disclosure, can be combined with a sterile aqueous carrier prior to administration. The pH of the pharmaceutical compositions typically will be between 3 and 11 (e.g., between about 5 and 9) or between 6 and 8 (e.g., between about 7 and 8). The resulting compositions in solid form can be packaged in multiple single dose units, each containing a fixed amount of the above-mentioned agent or agents, such as in a sealed package of tablets or capsules.

### Articles of Manufacture

The composition described herein can be packaged in a suitable container labeled, for example, for use as a therapy to treating or preventing rheumatoid arthritis or any of the methods disclosed herein. Accordingly, packaged products (e.g., sterile containers containing the composition described herein and packaged for storage, shipment, or sale at concentrated or ready-to-use concentrations) and kits, including at least one or more of the syndecan-1 inhibitors or the syntenin-1 inhibitors as described herein and instructions for use, are also within the scope of the disclosure. A product can include a container (e.g., a vial, jar, bottle, bag, or the like) containing the composition described herein. In addition, an article of manufacture further may include, for example, packaging materials, instructions for use, syringes, buffers or other control reagents for treating or monitoring the condition for which prophylaxis or treatment is required. The product may also include a legend (e.g., a printed label or insert or other medium describing the product's use (e.g., an audio- or videotape)).

The legend can be associated with the container (e.g., affixed to the container) and can describe the manner in which the compound therein should be administered (e.g., the frequency and route of administration), indications therefor, and other uses. The compositions can be ready for administration (e.g., present in dose-appropriate units), and may include a pharmaceutically acceptable adjuvant, carrier or other diluent. Alternatively, the compositions can be provided in a concentrated form with a diluent and instructions for dilution.

### Examples

It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1. Syntenin-1-Mediated Arthritogenicity is Advanced by Reprogramming RA Metabolic Macrophages and Th1 Cells

Materials and Methods. Experimental Design. This study aimed to decipher the role of Syntenin-1 and SDC-1 in inflammation and metabolism in RA immune cells and Syntenin-1-induced arthritis mouse model. To ensure a robust and unbiased experimental design, samples were obtained from RA patients or mice of both genders. Mice used within the same experimental group were age- and sex-matched. Rigor and reproducibility were maintained through well-powered studies and multiple distinct approaches to confirm the results. Power was calculated using parameters of α=0.05, and power=90% based on previous studies (Umar S, et al. Cell Mol Immunol 2021; 18:2199-2210; and Van Raemdonck K, et al. Arthritis Rheumatol 2021; 73:2003-2014). No outliers were excluded. Biological replicates are specified in each figure legend.

Human peripheral blood. Peripheral blood samples from RA patients were collected. RA patients were diagnosed according to the 1987 revised criteria of ACR (Arnett FC. et al. Arthritis Rheum 1988; 31:315-324). Patients gave written informed consent before blood was drawn. Patient information was de-identified; therefore, sex, age, treatment regimen, and demographic information are not known. Peripheral blood mononuclear cells (PBMCS) were isolated by density gradient centrifugation using Ficoll-Paque PREMIUM (GE Healthcare) and subsequently used for further analysis. Monocytes or T cells were negatively selected using the EasySep Human Monocyte Isolation Kit or the EasySep Human T cell enrichment Kit (both STEMCELL Technologies) according to the manufacturer's instructions.

Human synovial fluid (Arthrocentesis). Synovial fluids from RA and OA patients were obtained. RA patients were diagnosed according to the 1987 revised criteria of ACR (Arnett F C, et al. Arthritis Rheum 1988; 31:315-324). Patients gave written informed consent before blood was drawn. Patient information was deidentified; therefore, sex, age, treatment regimen, and demographic information are not known. Synovial fluid was collected by penetration of the joint space of the knee and subsequent aspiration of the fluid.

Animal studies. Wild type (WT) C57BL/6 mice (≥8 weeks old; Jackson Laboratory, Bar Harbor, Maine, USA) were bred in-house. SDC-1^{-/-} mice were generated (Alexander C M, et al. Nat Genet 2000; 25:329-332). Animals were housed in sterile static micro isolator cages on autoclaved corncob bedding with water bottles in a specific-pathogen-free (SPF) facility. Animal food is irradiated and water is autoclaved. Both food and water are provided ad libitum. The standard photoperiod for rodent rooms is 14 hours of light and 10 hours of darkness. Animals were provided with autoclaved nesting materials. Cages are changed at least weekly in either a biosafety cabinet or a HEPA-filtered animal transfer station. Eight- to twelve-week-old WT and SDC-1^{-/-} mice were injected intra-articularly with adenovirus (ad)-ctrl or ad-Syntenin-1 (3×10¹⁰ viral particles/ankle, Welgen) on days 0, 7, and 14. Joint circumference was assessed by a caliper and mice were sacrificed on day 15. Ankles were harvested and used for further analysis.

Quantitative Real-Time PCR. RNA was isolated using a TRIzol reagent (Life Technologies) according to the manufacturer's instructions. Transcription to cDNA and subsequent quantitative real-time PCR analysis was performed using the High-Capacity cDNA Reverse Transcription Kit (Applied Biosystems) and TaqMan Gene Expression Master Mix (Applied Biosystems). Predesigned IDT primers or TaqMan gene expression assays were used. Data are presented as fold change (2^{-ΔΔct}) normalized to the housekeeping gene (actin) and compared to the control. Data were acquired with the QuantStudio5 (Applied Biosystems) qRT-PCR device.

Western blot analysis. Samples were lysed in RIPA buffer (Cell Signaling Technology) supplemented with protease and phosphatase inhibitors (Roche laboratories) and protein concentration was assessed with the Pierce BCA Protein Assay Kit (ThermoFisher Scientific) following the manufacturer's instructions. Lysates were run on 10% polyacrylamide gels. Blotting was performed with the Trans-Blot Turbo Transfer System (Bio-Rad Laboratories). Samples were subsequently probed for SYN1 (1:1000, Aviva Systems Biology), SDC-1 (1:1000, Abcam, Cambridge, USA), p-Src, p-AKT, p-STAT1, p-STAT3, p-p38, p-ERK, p-JNK, IκBα, GLUT1, HK2, PFK2, LDHA, (1:1000, Cell Signaling Technology), mTOR (1:1000, Santa Cruz), actin (1:3000, Santa Cruz), and anti-rabbit IgG 25 HRP-linked or anti-mouse IgG HRP-linked (both 1:1000, both Cell Signaling Technology). Detection was performed using the iBright 1500 (Invitrogen by ThermoFisher Scientific).

Th1/Th17 cell differentiation. Human peripheral blood mononuclear cells were differentiated into Th1 or Th17 cells for 3 days in 10% FBS RPMI media in the presence of anti-CD3 and anti-CD28 (both 0.25 μg/ml, BioLegend Inc.). For Th1 and Th17 cell differentiation, cell media was supplemented with rhIL-12 (10 ng/ml, BioLegend) and rhIL-1β, rhIL-6, and rhTGF-β (20 ng/ml and 4 ng/ml, respectively), respectively.

Flow cytometry. Depending on the assay negatively selected human monocytes or human peripheral blood mononuclear cells from Rheumatoid Arthritis patients were stained with anti-CD14, anti-CD86, anti-CD206, anti-CD4 (BioLegend Inc.), or GLUT1 (Novus Biologicals) fluorescently labeled antibodies. Prior to intracellular staining, cells were stimulated with PMA (100 ng/ml) and ionomycin (1.5 μM; both Sigma-Aldrich) in the presence of Brefeldin A (eBioscience, San Diego, USA) for 3-4 h. To exclude dead cells, cells were stained by the ZombieViolet Fixable Viability Kit (BioLegend Inc.). For intracellular staining, cells were fixed and made permeable by the Cyto-Fast Fix/Perm Buffer Set (BioLegend Inc.) and subsequently stained with anti-IFNγ and anti-IL-17 (both eBioscience) fluorescently labeled antibodies. Data were acquired at the Flow Core Facility at the University of Illinois at Chicago using the Gallios 10/3 flow cytometer (Beckman Coulter).

Syntenin-1 stimulation and inhibition. RA monocytes were differentiated into macrophages (MΦ) for 2 days in 10% RPMI. On day 3. MΦs were either untreated (PBS) or treated with Syntenin-1 (1000 ng/ml, NKMAX Co.) for 6 h to 48 h. For Syntenin-1 inhibition, MΦs were starved overnight in the presence of 2-deoxy-D-glucose (2-DG; 5 mM, Sigma-Aldrich, St. Louis, USA), hypoxia-inducible factor 1α inhibitor (HIF1αi; 2 μM, Calbiochem), mTOR inhibitor (mTORi; 1 μM: Everolimus, Sigma-Aldrich), human IL-5R antibody (IL5Ra; 2 μg/ml. R&D Systems), PDZ1 domain inhibitor peptide (PDZ1; 10 μM, Tocris Bioscience), IL-12 antibody (IL-12ab; 10 μg/ml, BioLegend) or SDC-1 antibody (SDCab; 1:100, Diaclone) following Syntenin-1 (1000 ng/ml, NKMAX Co.) stimulation for 6 h or 24 h. Cells were subsequently harvested in TRIzol reagent (Life Technologies) or RIPA buffer (Cell Signaling Technology) for mRNA quantification and western blot analysis; conditioned media was collected for ELISA.

Preosteoclast differentiation. RA monocytes were differentiated into preosteoclasts for 7 days in 10% FBS WMEM media in the presence of RANKL and M-CSF (both 10 ng/ml; suboptimal condition). On day 7 cells were either untreated (PBS) or treated with Syntenin-1 (1000 ng/ml, NKMAX. Co) for 6 h and subsequently harvested in TRIZol reagent (Life Technologies) to assess mRNA transcription of osteoclastic factors.

Seahorse ATP Rate Kit. Glycolytic ATP production (glycolysis) and mitochondrial ATP production (oxidative phosphorylation) were measured using the Seahorse XF ATP Rate Test kit (Agilent Technologies), according to the manufacturer's instructions. RA monocytes (2×10⁵ cells/well) were cultured for 2 days and Syntenin-1 (1000 ng/ml) and PBS were injected during the experiment. Percent glycolysis increase and % oxidative phosphorylation decrease were calculated by the following equation:

% Glycolysis (INDUCED)−% Glycolysis (BASAL)=% glycolysis increase.

Metabolite quantification. The concentration of the glycolytic metabolite lactate was measured in conditioned media using the L-Lactate Assay Kit (Sigma-Aldrich, St. Louis, USA) following the manufacturer's instructions.

Enzyme-Linked-Immunosorbent-Assay (ELISA). Human Syntenin-1, CCL2, IL-6, IL-12, IL-18, and TNF-α protein levels were quantified by ELISA according to the manufacturer's instructions (R&D Systems, Minneapolis, MN).

Immunohistochemistry. Formalin-fixed, paraffin-embedded human tissue samples were sectioned. Normal, OA, and RA ST samples were stained to quantify Syntenin-1 presentation. Staining was scored on a scale of 0-5 in a blinded manner (0=normal appearance, 1=minimal changes, 2=mixed appearance, 3=moderate changes, 4=marked changes, and 5=severe changes) (Umar S, et al. Cell Mol Immunol 2021; 18:2199-2210)), and distinguished within the synovial lining, sub lining, and vasculature. Formalin-fixed mouse ankles were decalcified and paraffin-embedded. Slides were deparaffinized in xylene, and antigen retrieval was achieved. Mouse ankle sections were stained for H&E, F4/80 (1:100, GeneTex, Irvine, CA), inducible nitric oxide synthase (iNOS; 1:200, Santa Cruz Biotechnology. Dallas. TX), arginase 1 (1:200, Santa Cruz Biotechnology), GLUT1 (1:100, Cell Signaling Technology), HIF1α (1:50, Santa Cruz Biotechnology), cMYC (1:50, Novus Bio), mTOR (1:50, Santa Cruz Biotechnology), and CD3 (1:100, GeneTex). Staining was scored for inflammation, synovial lining thickness, and bone erosion on a 0-5 scale at ×100 magnification.

RNASeq transcriptome analysis of the Pathobiology of Early Arthritis Cohort (PEAC). The web interface peac.hpc.qmul.ac.uk/ developed by Lewis et al. (Lewis M J, et al. Cell reports 2019; 28:2455-2470 e2455) was used to correlate the expression of Syntenin-1 and SDC-1 in blood and synovial biopsies from individuals with early rheumatoid arthritis against clinical parameters. Methods for the generation of data used in this web interface are known (Lewis M J, et al. Cell reports 2019: 28:2455-2470 e2455; and Humby F, et al. Ann Rheum Dis 2019; 78:761-772). Gene transcript expression levels are expressed as VST (variance stabilizing transformation) transformed read counts using the Bioconductor package DESeq2. Synovial histology was scored using a semiquantitative grading from 0-4 (Humby F, et al. PLoS Med 2009; 6:e1). A fuller description and reference atlas for histology markers is provided in the Supplementary appendix in Humby et al. 2019 (Humby F, et al. Lancet 2021; 397:305-317). The raw RNA-Seq data has been deposited at ArrayExpress accession E-MTAB-6141.

Statistical Analysis. For comparison among multiple groups, one-way ANOVA followed by Tukey's multiple comparison test was employed, using Graph Pad Prism9 software. The data were also analyzed using a two-tailed Student's t-test or Mann-Whitney test for paired or unpaired comparisons between two groups. When comparing RNA-Seq data against continuous or ordinal variables, the Spearman rank correlation test was used, and Spearman rho and p-values are shown. p<0.05 was considered statistically significant.

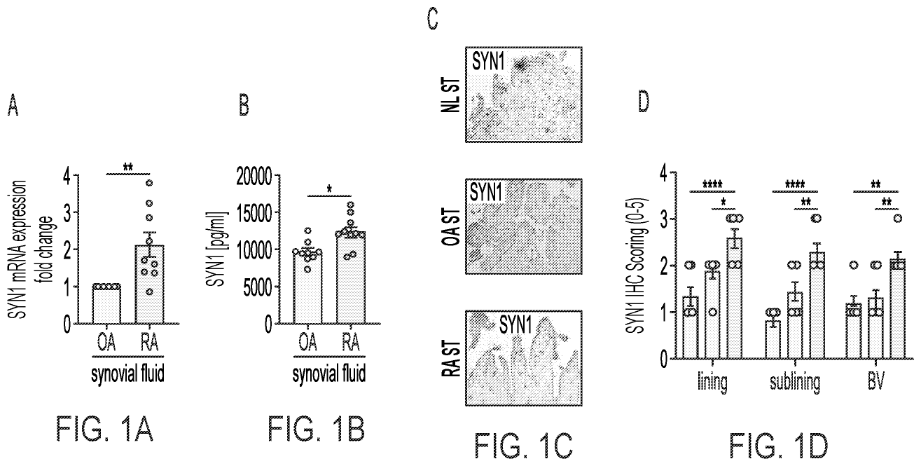

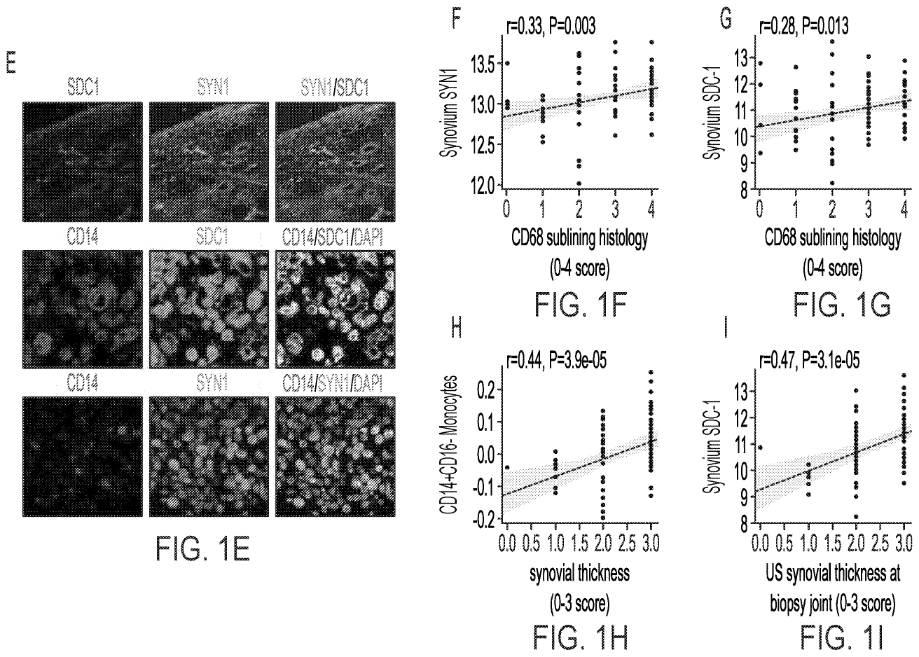

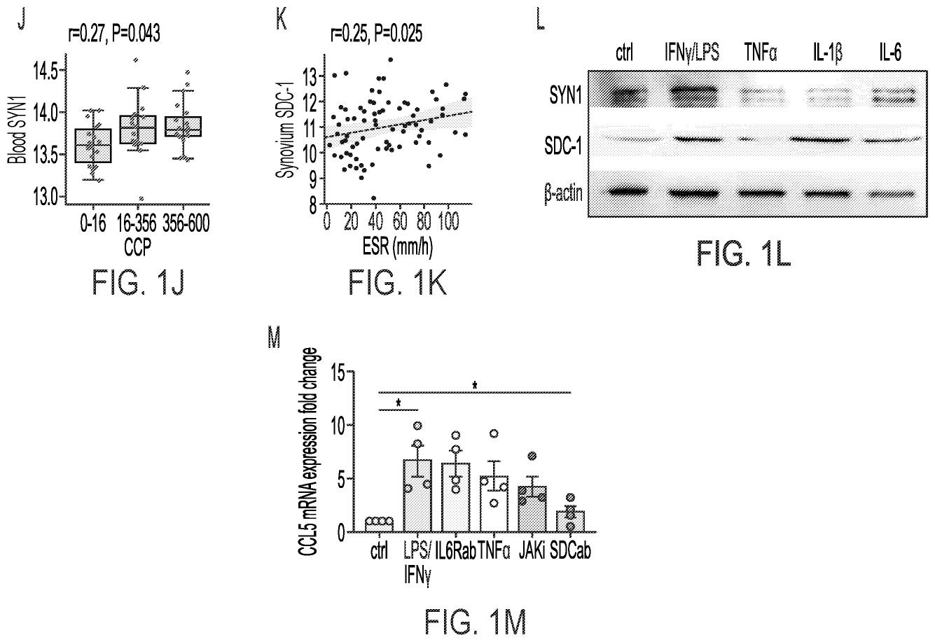

Results. Syntenin-1 protein levels are enriched in RA compared to OA synovial, luid and its expression in RA synovial tissue or circulation is linked to MΦ markers and clinical manifestation. Through RNAseq analysis, it was found that blood Syntenin-1 transcriptome is linked to cyclic citrullinated peptide (CCP) antibodies (Gotte M, et al. Invest Ophthalmol Vis Sci 2002:43:1135-41), hence its expression and immunoregulation were characterized in RA relative to osteoarthritis (OA) and NL counterparts. It was also found that Syntenin-1 mRNA and protein levels are significantly amplified in RA compared to OA synovial fluid (FIGS. 1A and 1B). Morphological studies exhibit that in RA, the lining and sub-lining cells, as well as blood vessels, are the primary sources of Syntenin-1 release relative to NL STs (FIGS. 1C and 1D). Immunofluorescence staining authenticated that Syntenin-1 and SDC-1 are expressed on RA ST CD14⁺MΦs (FIG. 1E). In parallel, Syntenin-1 (p=0.003, r=0.33) and SDC-1 (p=0.013, r=0.28) transcript levels were linked to the number of CD68⁺MΦs quantified by histology in RA synovial tissues (FIGS. 1F and 1G). Interestingly, CD14⁺CD16⁻ myeloid cells (p<0.001) and SDC-1 transcript expression (p<0.001) were associated with RA ultrasound-guided synovial tissue thickness (FIGS. 1H and 1I).

Consistent with the importance of Syntenin-1 and SDC-1 in RA pathophysiology, circulating Syntenin-1 and synovial tissue SDC-1 transcription levels are linked to clinical parameters such as the cyclic citrullinated peptide (CCP) and erythrocyte sedimentation rate (ESR) (FIGS. 1J and 1K). Notably, the classical inflammatory mediators, LPS/IFNγ, mutually upregulate the expression of Synentin-1 and SDC-1 in human myeloid cells (FIG. 1L). However, in myeloid cells, SDC-1 protein levels are also distinctly escalated by IL-I1β and IL-6 exposure (FIG. 1L).

The data show that LPS/IFNγ-induced CCL5 transcription is suppressed by SDC-1 antibody (SDCab) in contrast to IL-6R Ab, TNFi or Jaki (Tofacitinib) therapy (FIG. 1M). Overall, the data show that Syntenin-1 and SDC-1 overexpression in RA macrophages is associated with clinical features, which can modulate RA pathology in comparison to current biotherapies.

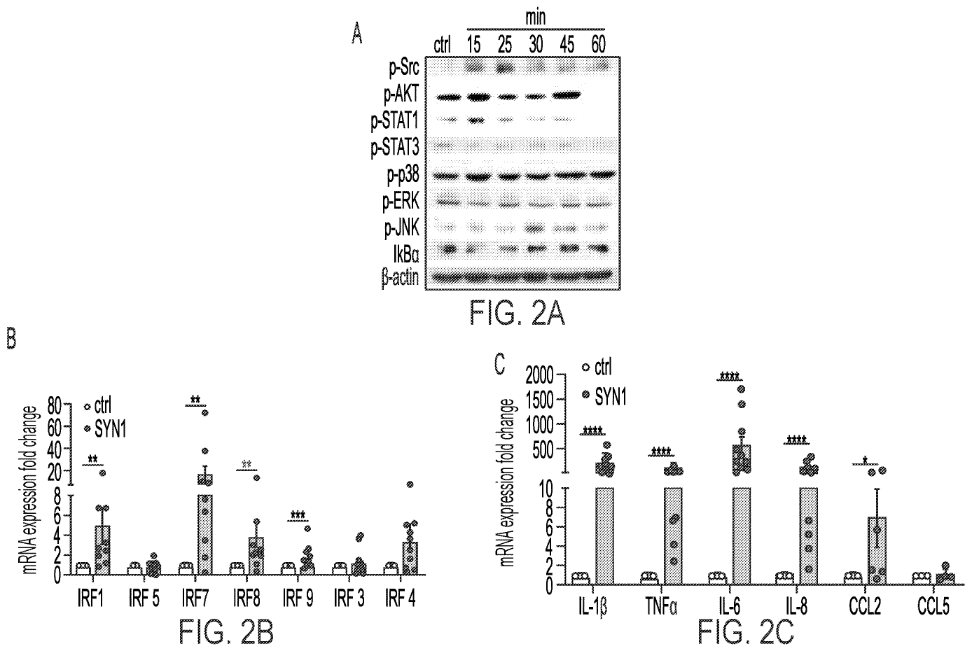

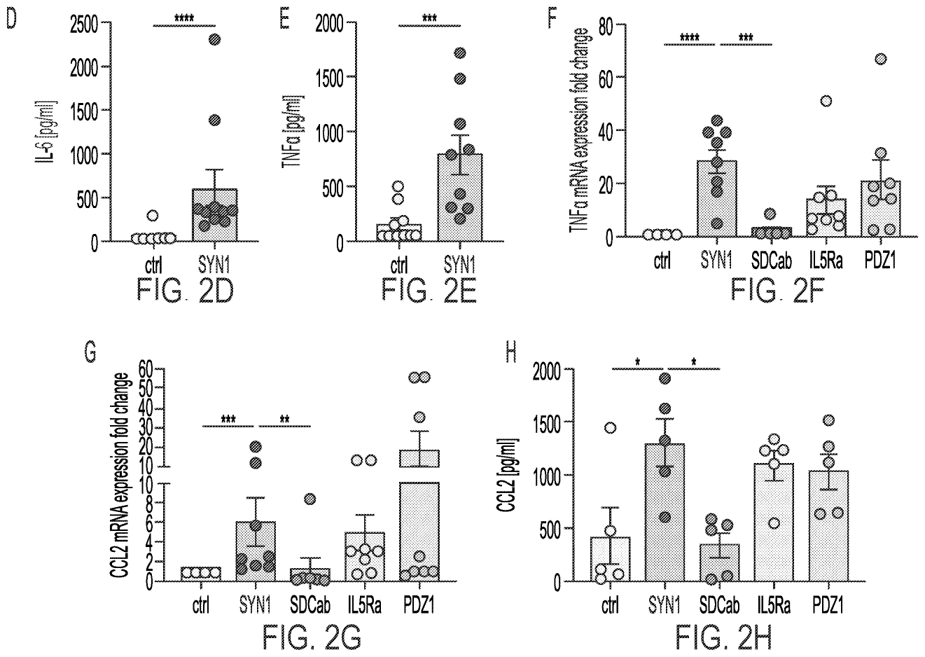

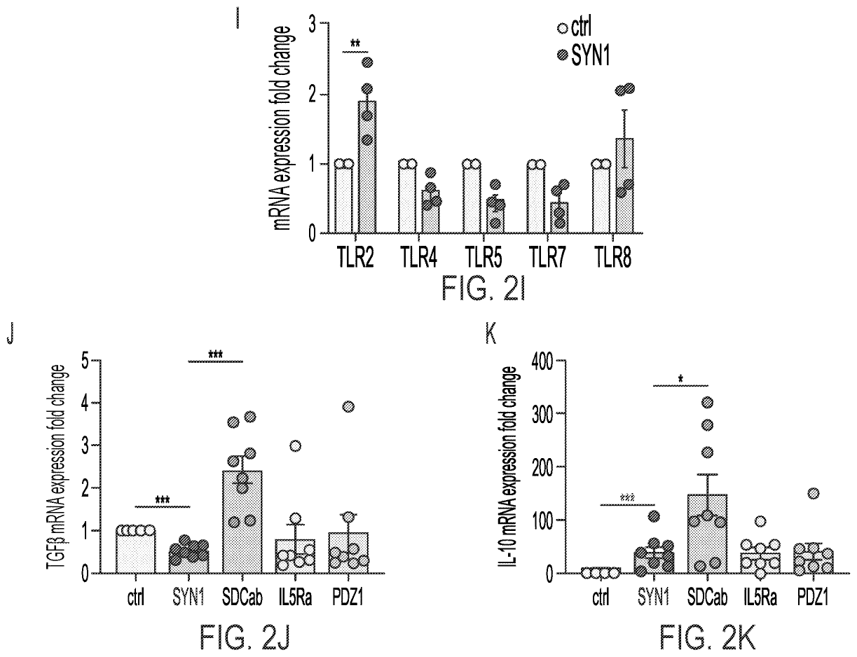

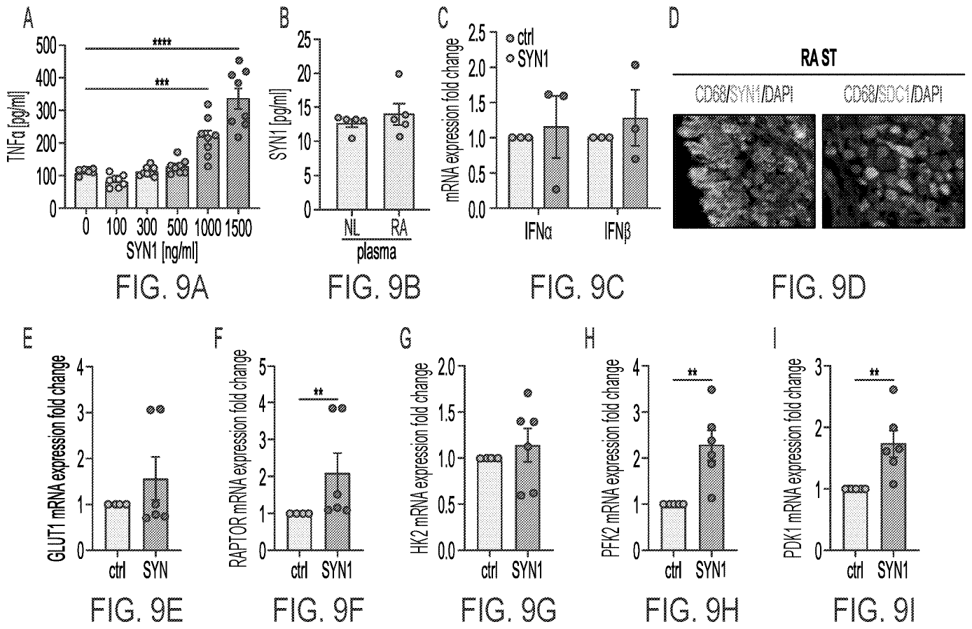

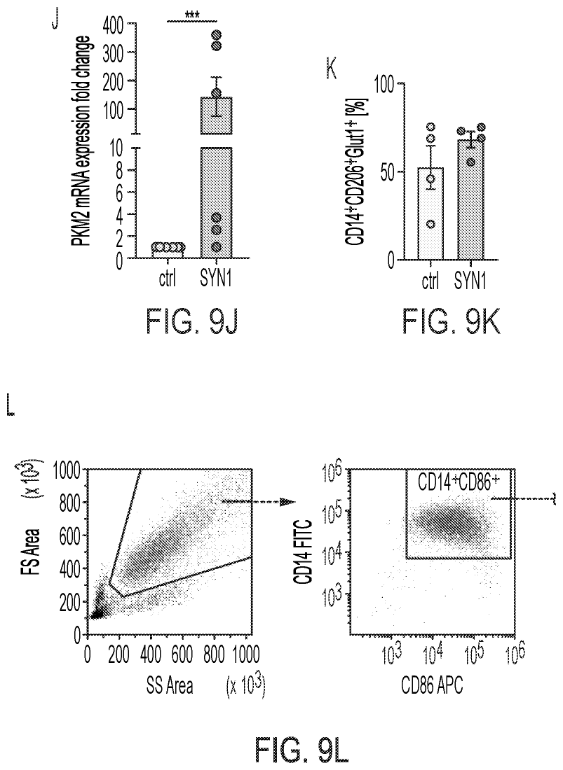

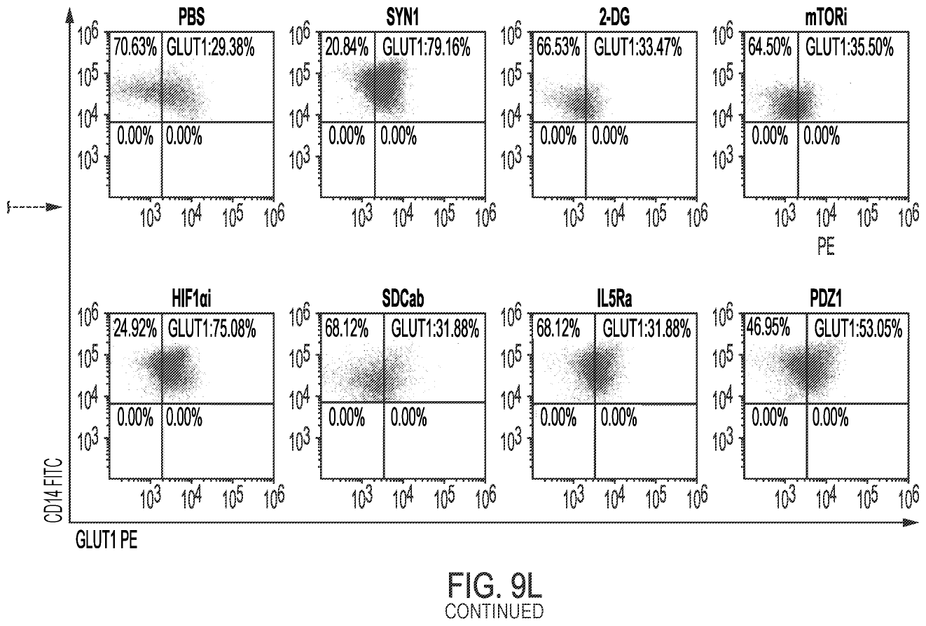

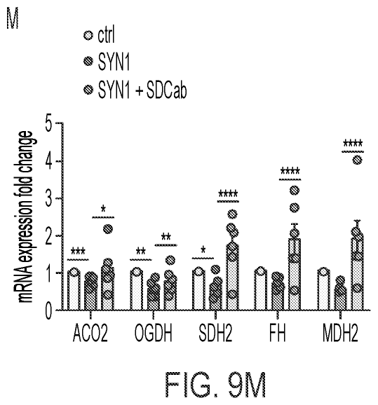

Syntenin-1 ligation to SDC-1 advances RA MO inflammatory imprint independently of IL-5R or PDZ1 function. To elucidate the significance of Syntenin-1/SDC-1 in RA pathogenesis, their signaling pathways and inflammatory profile were characterized in MΦs. Syntenin-1 was carefully titrated in RA MΦs, and the effective dose was based on its TNFα induction (FIG. 9A). Human monocytes exposed to Syntenin-1 showed phosphorylation of SRC, protein kinase B (AKT), Signal Transducer And Activator Of Transcription-1 (STAT1), and c-Jun N-terminal kinase (JNK) pathways as well as degradation of IκB (FIG. 2A). However, STAT3, p38, and ERK signaling were unchanged in myeloid cells stimulated by Syntenin-1 (FIG. 2A). In RA MΦs reconfigured by Syntenin-1, the wide range of activated signaling pathways, was in concert with expansion of inflammatory interferon transcription factors, IRF1, IRF7, IRF8, and IRF9 as well as monokines, IL-1, TNF-α, IL-6, IL-8, and CCL2 (>7× fold) (FIGS. 2B to 2E). On the contrary, the pro-repair transcription factors, IRF3 and IRF4, were uninvolved in Syntenin-1-differentiated RA MΦs (FIG. 2B). Remarkably, while elevated levels of TNF-α, and CCL2 were intercepted by SDC-1 Ab therapy, use of IL-5R Ab or PDZ1 inhibitor (1) was ineffective in this process (FIGS. 2F to 2H). Further, while TLR2 was exclusively amplified in Syntenin-1-polarized RA MΦs, TLR4/5/7/8 transcription remained unchanged (FIG. 2I). In contrary to IL-5R Ab or PDZ1 i therapy in RA MΦs, transcriptional regulation of the pro-repair factors by Syntenin-1 was accentuated via SDC-1 Ab (FIGS. 2J and 2K). Taken together, an extensive array of signaling pathways, transcription factors, and monokines are involved in the remodeling of RA MΦs through Syntenin-1/SDC-1 leading to a misbalanced pro-inflammatory over the pro-repair network.

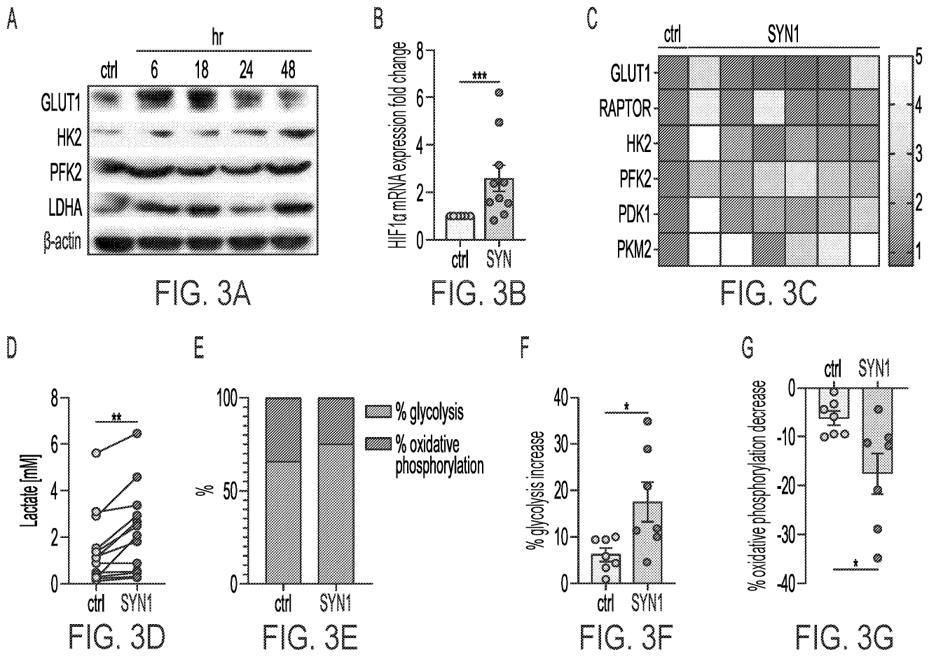

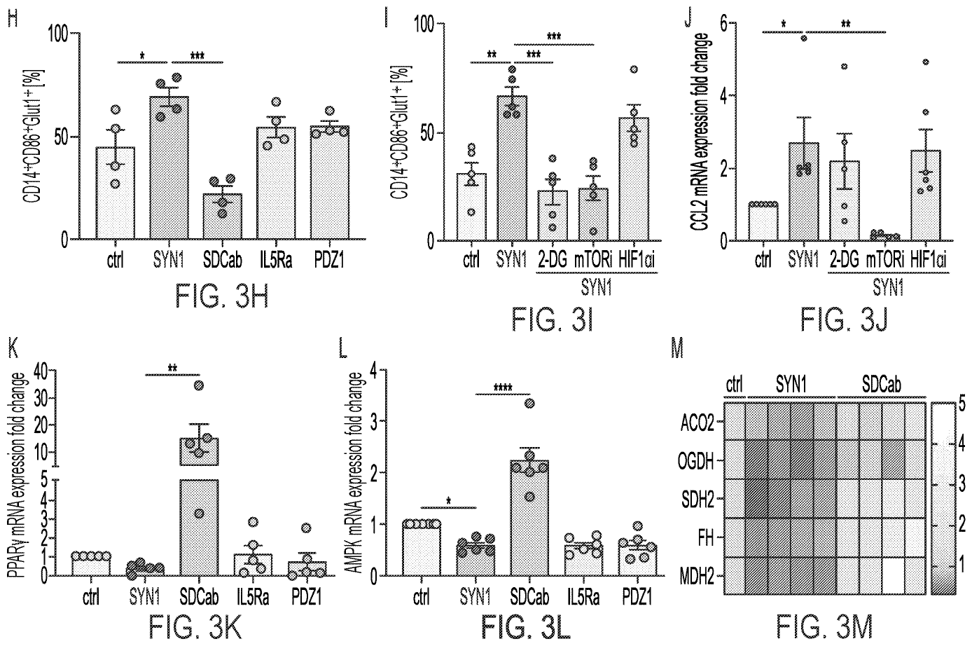

Syntenin-1 and SDC-1 escalate RA MO metabolic reprogramming. It has been shown that in the inflammatory landscape of RA, MΦs can be restrained by glucose uptake inhibition (Jaiswal A K, et al. J. i. 2018; 201:1651-61; Kim W Y, et al. Exp Mol Med. 2014; 46:e90; Lewis M J, et al. Cell Rep 2019; 28:2455-70; and Van Raemdonck K, et al. Immunol Cell Biol. 2022; 100:127-35). Hence, experiments were conducted to determine whether the Syntenin-1-potentiated inflammatory phenotype in RA MΦs is influenced by glycolytic rewiring. In parallel to amplifying inflammatory responses, RA MΦs differentiated by Syntenin-1 display elevated GLUT1, HK2, HIF1α, RAPTOR, and PKM2 expression (FIGS. 3A to 3C, FIGS. 9D to 9I). Additionally, higher protein expression of LDHA and lactate in Syntenin-1-reprogrammed RA MΦs was supported by ATP being mainly generated through glycolysis (Q % glycoATP) over oxidative phosphorylation (←% mitoATP) (FIGS. 3A, 3D to 3G). The data show that Syntenin-1 reconfigures naïve cells into RA CD14⁺CD86⁺GLUT1⁺MΦs through SDC-1 binding, while blockade of IL-5R or PDZ1 was inconsequential on this function (FIG. 3H). Particularly, the differentiation of glycolytic CD14⁺CD86⁺GLUT1⁺MΦs by Syntenin-1 and its ability to promote inflammatory monokines, including CCL2, was dependent mTOR1 activation, however, this mechanism of action was independent of HIF1α signaling (FIGS. 3I-3J). Despite the inefficacy of Syntenin-1 on CD14⁺CD206⁺GLUT1⁺MΦs frequency (FIG. 9K), downregulation of oxidative intermediate (AMPK) and enzymes (ACO2, OGDH, SDH2, FH, MDH) in these RA myeloid cells were reversed by SDC-1 Ab (FIG. 3K to 3M). In short, the inflammatory imprint of Syntenin-1-differentiated RA MΦs is interconnected to its metabolic activity through SDC-1 ligation and RAPTOR/mTOR signaling.

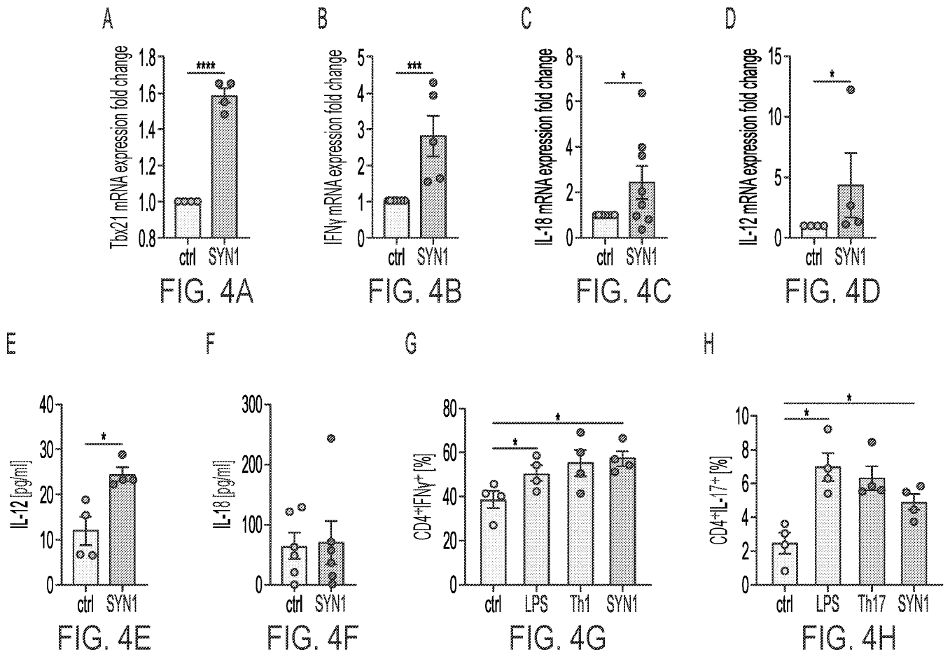

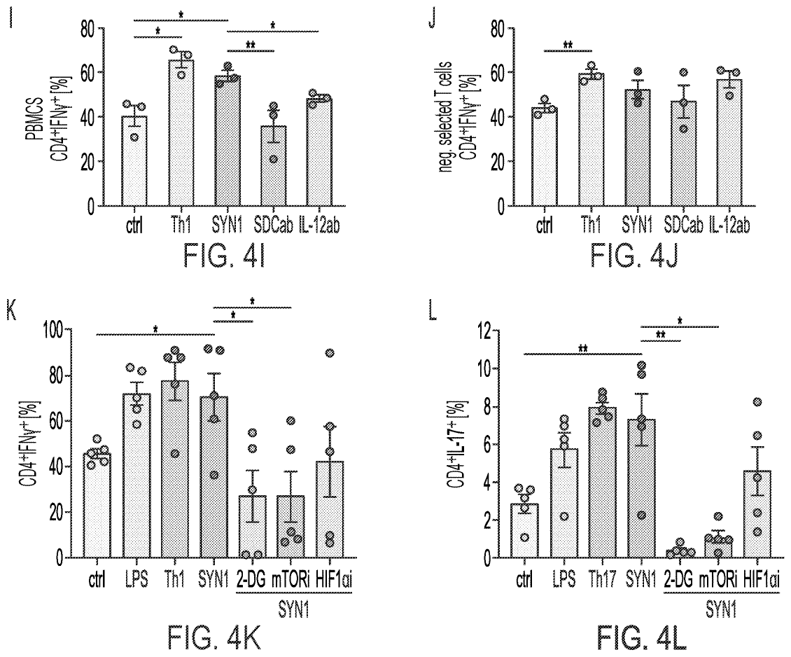

RA MΦs remodeled by Syntenin-1 promote Th1 and Th17 cell differentiation. Next, the impact of Syntenin-1 was characterized on T effector cell differentiation in RA patients. RA peripheral blood mononuclear cells (PBMCs) exposed to Syntenin-1 displayed a strong Th1 profile by transcriptionally upregulating Tbx21/T-bet, IFNγ, IL-18, and IL-12 (FIGS. 4A to 4F). Flow cytometry analysis validated that, similar to LPS and IL-12, Syntenin-1 polarizes RA naive cells into the Th1 subtype (FIGS. 4G and 4H). While IL-18 was undetected in the conditioned media generated from Syntenin-1-activated Th1 cells, detection of IL-12 validated that it is predominately responsible for the polarization of Tbx21⁺IFNγ⁺Th1 cells by Syntenin-1. As shown herein, anti-IL-12 Ab therapy could impair syntenin-1-mediated Th1 cell polarization in RA PBMCs but was ineffective in T cell culture alone (FIGS. 4I, 4J). In parallel, the presence of myeloid cells in RA PBMCs can further expand syntenin-1-induced Th1 differentiation compared with T cell culture (FIGS. 4I, 4J). Like Th1 cells, RA PBMCs exposed to Syntenin-1 were differentiated into Th17 cells, in part through glucose uptake and mTOR signaling and independent of the HIF1α pathway (FIGS. 4G to 4K). Altogether, the results demonstrate that activation of glycolysis via mTOR is responsible for Syntenin-1-instigated metabolic RA MO reconfiguration and its cross-regulation of Th1 and Th17 cell development.

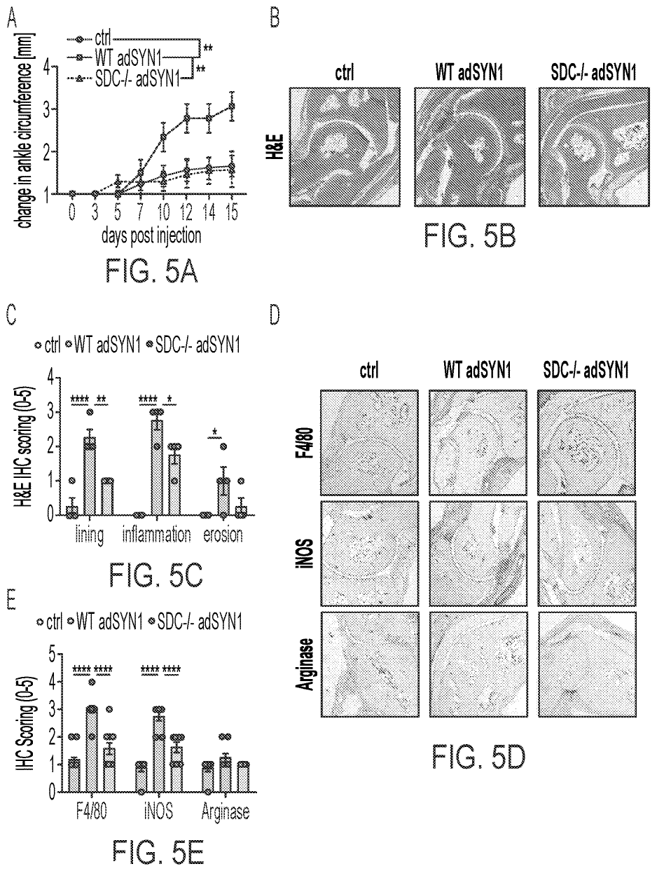

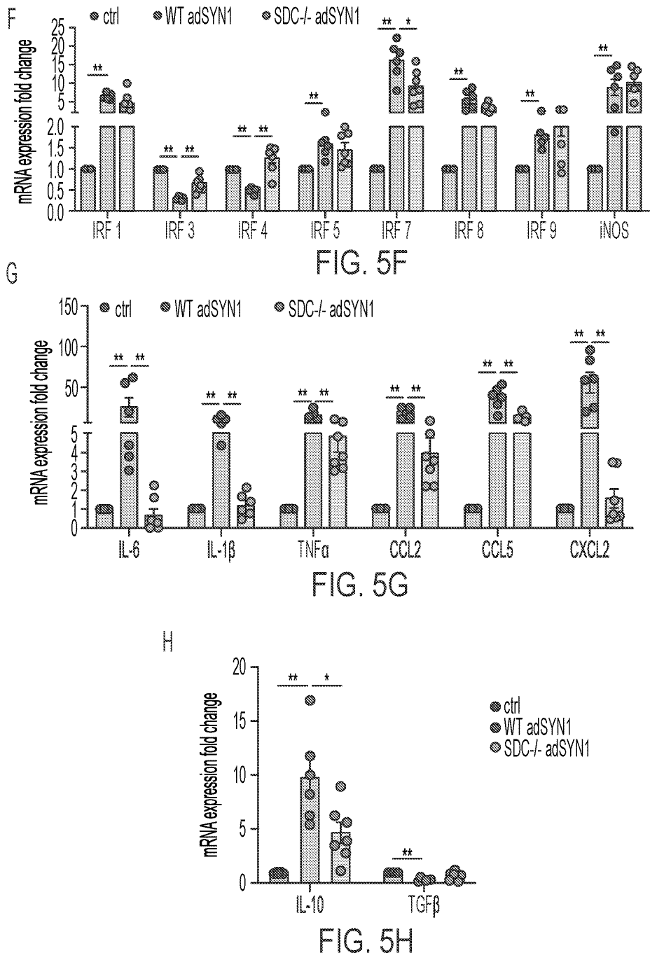

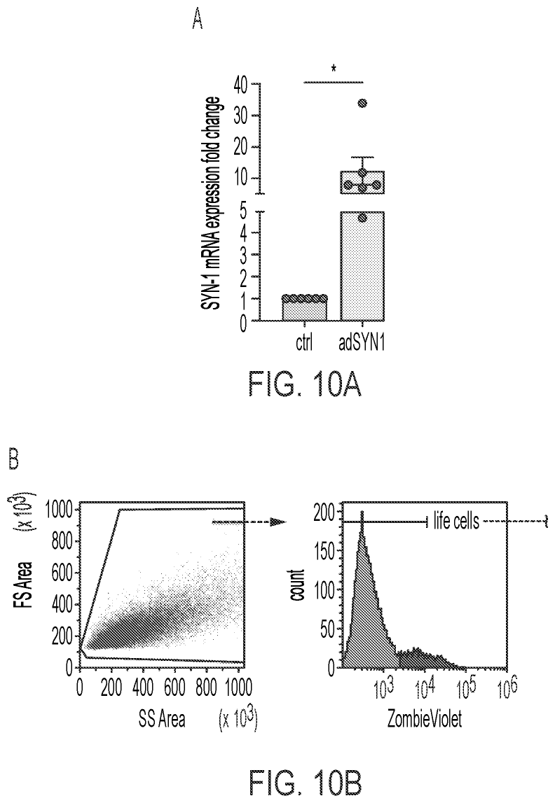

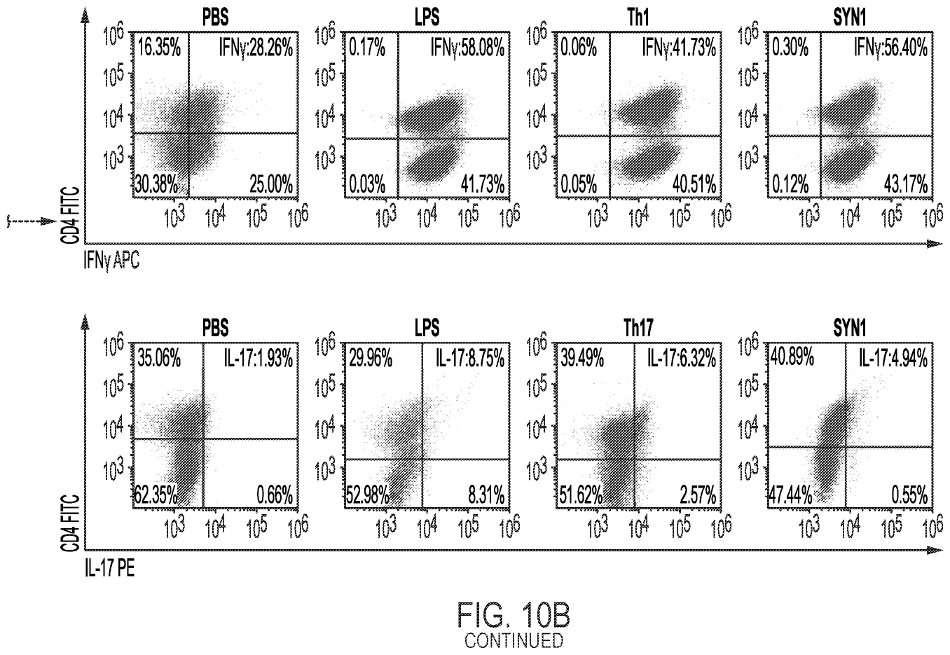

SDC-1 deficient mice are resistant to Syntenin-1-mediated arthritis. To evaluate the arthritogenic potential of Syntenin-1, adenovirus (ad) expressing Syntenin-1 was intra-articularly injected into wild-type (WT) mice compared to Ad-Control (ctrl). Local injection of Syntenin-1 progressively increased ankle circumference up to day 12, subsequently, joint swelling plateaued until day 15 when mice were sacrificed (FIG. 5A; FIG. 10A). In line with these observations in RA cells, Syntenin-1-induced arthritis was attenuated in SDC-1^{-/-} mice (FIGS. 5A and 5B). Consistently, joint lining thickness, inflammation, and bone erosion, advanced in WT by Syntenin-1-induced arthritis, were dysregulated in SDC-1^{-/-} mice (FIGS. 5B and 5C). While F480⁺iNOS⁺MΦs were responsible for the arthritogenicity escalated by Syntenin-1 and the pathology was disrupted in SDC-1^{-/-} relative to WT animals, F4/80⁺arginase⁺MΦs were unaffected in this process (FIGS. 5D and 5E). Local Syntenin-1 expression represented RA MΦ differentiation by displaying a diverse expansion of inflammatory IRF1, IRF5, IRF7. IRF8, and IRF9 (up to a 15-fold increase) as well as monokines, IL-6. IL-1β, TNF-α. CCL2, CCL5, and CXCL2 (up to 50-fold increase), which were diminished in SDC-1^{-/-} mice (FIGS. 5F and 5G). Inversely, the joint pro-repair mediators, IRF3, IRF4, and TGFβ were suppressed by ectopic Syntenin-1 expression (FIGS. 5F and 5H). However, distinct from SDC-1 neutralizing Ab, SDC-1^{-/-} mice were unable to replenish joint IL-1β or TGFβ transcription in Syntenin-1 arthritic animals (FIG. 5H). Collectively, the data reveal that SDC-1 deficiency reverses the arthritic F4/80⁺iNOS⁺MΦreprogramming by Syntenin-1, without influencing the pro-repair F4/80⁺arginase*MD imprint.

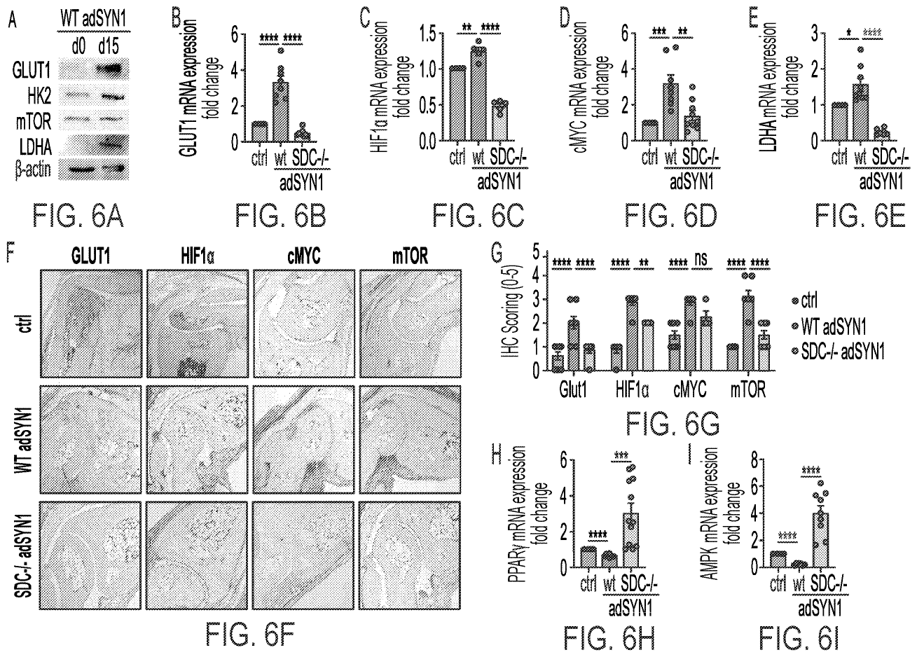

Syntenin-1-induced arthritis is manipulated by the joint hypermetabolic activity. To further characterize arthritis promoted by Syntenin-1, joint immunometabolism was investigated in naïve compared to arthritic mice. The results show that GLUT1, HK2, mTOR/p70, and LDHA protein expression were elevated in Syntenin-1 arthritic joints harvested at day 15 compared to non-arthritic counterparts (FIG. 6A). Further, overexpression of these glycolytic metabolites was authenticated by transcriptome and morphological analysis in WT mice locally expressing Syntenin-1 compared to SDC-1^{-/-} or non-arthritic animals. Particularly, GLUT1, HIF1α, cMYC, LDHA, and mTOR/p70 expression increased in Syntenin-1-induced arthritis, was subsided in SDC-1^{-/-} compared to WT mice (FIGS. 6B to 6G). In contrast, the downregulation of oxidative regulators, PPARγ and AMPK, by Syntenin-1 local expression in WT relative to non-arthritic mice was reversed in SDC-1^{-/-} animals (FIGS. 6H and 61). Overall, SDC-1 dysregulation mitigates Syntenin-1-mediated arthritogenicity by mainly normalizing the inflammatory and glycolytic networks and narrowly restoring the pro-repair or oxidative profile.

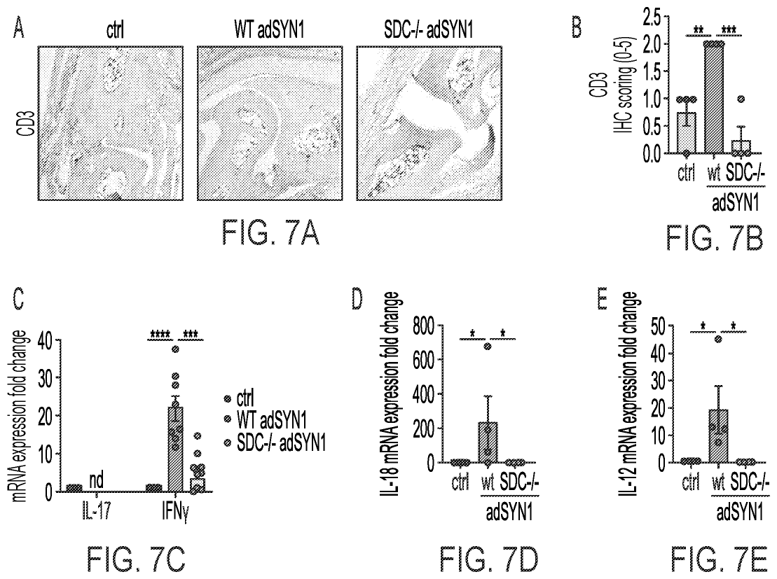

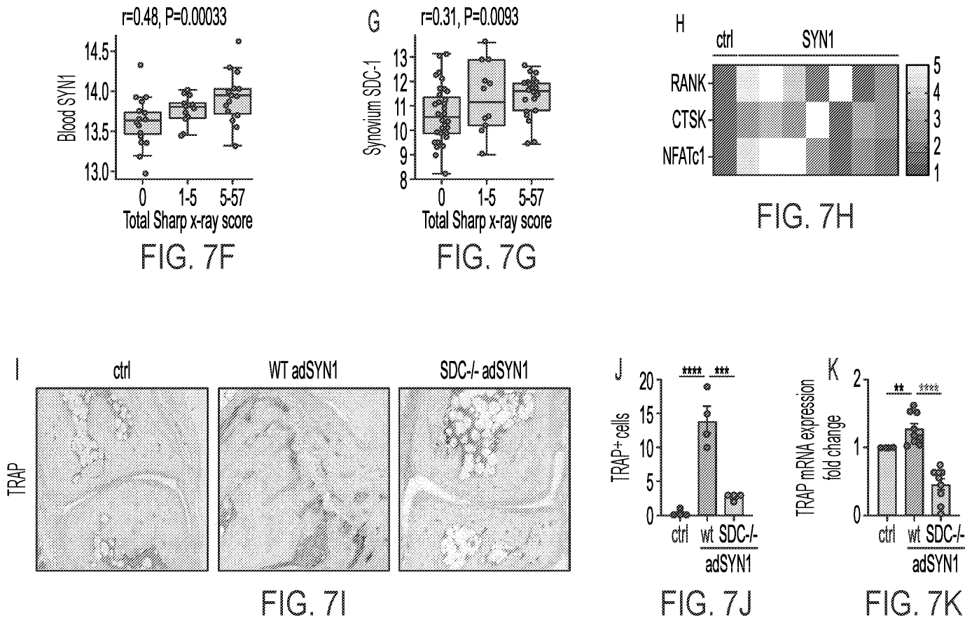

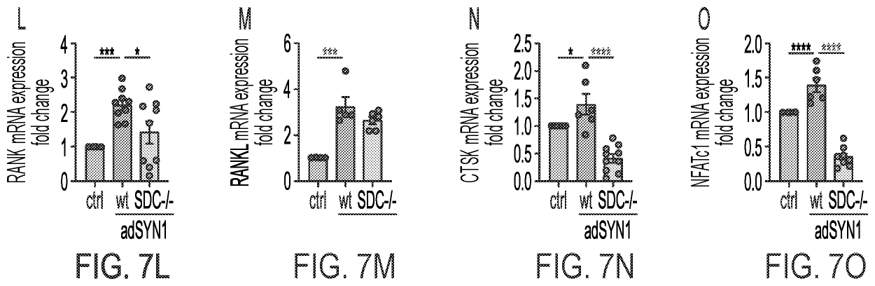

Arthritis potentiated by Syntenin-1 is influenced by CD3 T cell migration and Th1 cellpolarization. Given that RA PBMCs, exposed to Syntenin-1, were polarized into Th1 and Th17 cells, it was tested whether T cells play an important role in Syntenin-1-mediated arthritis. Intriguingly, local expression of Syntenin-1 attracts CD3⁺ T cells into the arthritic WT joints, which are significantly restrained in SDC-1^{-/-} mice (FIGS. 7A and 7B). Despite transcription of Th1 signature genes, IFNγ, IL-18, and IL-12 being highly elevated in WT Syntenin-1-arthritic mice and impaired in SDC-1-animals, Th17 cell polarization was unaffected (FIGS. 7C to 7E). Taken together, the RA and preclinical data emphasize that Syntenin-1 skews T naive cell reprogramming towards Th1 cells.

Syntenin-1 remodels RA preosteoclasts and arthritic joint cells into mature osteoclasts through an overlapping mechanism. Circulating Syntenin-1 (p<0.001, r-0.48) and synovial tissue SDC-1 (p=0.0093, r=0.31) transcript levels are linked to bone erosion evaluated in radiographic images of RA hands and feet by Sharp score (FIGS. 7F and 7G). The data herein shows that Syntenin-1 cultivates RA PBMCs into mature osteoclasts in part by activating the expression of several osteoclastic mediators, RANK, cathepsin K (CTSK), and NFATc1 (FIG. 7H). Correspondingly, the frequency of TRAP⁺ osteoclasts and transcription of RANK, CTSK, and NFATc1 were elevated in WT compared to SDC-1^{-/-} mice induced with Syntenin-1-mediated arthritis (FIGS. 71 to 7O). Altogether, the data underlines the significance of Syntenin-1/SDC-1 in transforming RA and murine precursor cells into mature osteoclasts by similar osteoclastic mediators.

Described herein is an endogenous regulator, Syntenin-1, that is released from classically differentiated inflammatory MΦs and its expression is unaffected by RA biotherapies. Transcriptome and morphological analysis exhibited that Syntenin-1 and its pathogenic receptor, SDC-1 are co-expressed on RA synovial tissue CD14⁺CD68⁺MΦs. Concurrently, Syntenin-1 and/or SDC-1 expression in RA blood or synovial tissue is closely linked to CCP levels, ESR, ultrasound detected synovial tissue thickness and bone erosion. Syntenin-1 advances RA CD14⁺CD86⁺GLUT1⁺MΦ reprogramming that displays dysregulated oxidative intermediates together with an extensive range of inflammatory IRFs, monokines, and glycolytic factors, that are counteracted by blockade of SDC-1, glucose uptake, and/or mTOR signaling. Recapitulating RA mechanism of function, IL-12 and/or IL-18 transcriptional upregulation in Syntenin-1 arthritic joints reconfigures the infiltrated T cells into Th1 cells. While in WT mice, Syntenin-1-triggered inflammatory, glycolytic, and erosive networks are abrogated in SDC-1^{-/-} animals, joint pro-repair monokines are unchanged and the oxidative metabolites are modestly replenished. Collectively, the results disclosed herein highlight that targeting the Syntenin-1/SDC-1 pathway can provide a strategy for deregulating RA metabolic misfunction.

Syntenin-1 is expressed in metastatic tumor cells in melanoma (Boukerche H, et al. Proc Natl Acad Sci USA 2008; 105:15914-9; and Boukerche H, et al. Cancer Res 2005; 65:10901-11), breast and lung cancer (Kim W Y, et al. Exp Mol Med. 2014; 46:e90; and Koo T H, et al. Oncogene 2002; 21:4080-8) as well as in glioma cells (Li Q, et al. PLoS One 2012; 7:e48278) regulating disease expansion in part by cell membrane motility (Shimada T, et al. Int J Mol Sci 2019:20. doi:10.3390/ijms20174171). Nonetheless, the cellular expression, immunomodulation, and pathobiology of Syntenin-1 are undescribed in RA patients and preclinical models. The results described herein show that Syntenin-1 is overexpressed in RA specimens compared to OA synovial fluid and NL synovial tissue, particularly, in MΦs and endothelial cells. Interestingly, a recent study has shown that Syntenin-1 protein levels were amplified in exosomes isolated from RA synovial fluid with higher disease activity compared to less severe counterparts (Foers A D, et al. Clin Transl Immunology 2020; 9:e1185). Others have exhibited that MΦ-derived exosomes contain Syntenin-1 (Garin J, et al. J Cell Biol 2001; 152:165-80), indicating that synovial fluid Syntenin-1 may be released from MD microvesicles. Concurrently. Syntenin-1 together with SDC-1 interacts with proteins responsible for exosome biogenesis to rearrange the extracellular vesicle cargo (Baietti M F, et al. Nat Cell Biol 2012; 14:677-85; and Zimmermann P, et al. Dev Cell 2005; 9:377-88).

Interestingly, in RA MΦs, SDC-1 controls the phosphorylation of the co-receptor, M-CSFR, when exposed to IL-34 (Van Raemdonck K, et al. Arthritis Rheumatol 2021; 73:2003-14). Consequently, SDC-1 is indispensable for IL-34-mediated arthritis by influencing M34 MD and osteoclast differentiation in part via joint hypermetabolic activity instigated by HIF1a and cMYC ((Van Raemdonck K, et al. Arthritis Rheumatol 2021; 73:2003-14; and Umar S, et al. Cell Mol Immunol. 2021:18:2199-210). Distinct from cancer cells, in RA MΦs, the binding partner of Syntenin-1 is restricted to SDC-1 since IL-5R is exclusively expressed in B cells, basophils, and eosinophils (Takatsu K. Proc Jpn Acad Ser B Phys Biol Sci 2011; 87:463-85; Denburg J A, et al. Int Arch Allergy Immunol 2001; 124:246-8; and Kabashima K, et al. Immunol Rev 2018; 282:114-20) and its blockade does not impact the function of Syntenin-1.

While stimulation with LPS/IFNγ mutually upregulates Syntenin-1 and SDC-1 protein levels, SDC-1 is modulated by IL-1β and IL-6 activation in human myeloid cells. Similarly, in SV40-immortalized melanoma cells, Syntenin-1 levels are highly responsive to IFNγ stimulation (Lin J J, et al. Gene 1998; 207:105-10). Inversely, SDC-1 is differentially regulated by TGF-β and bFGF in various cell types (Cizmeci-Smith G and Carey D J. Arterioscler Thromb Vasc Biol 1997; 17:2609-16). Moreover, soluble SDC-1 detected in RA sera as a result of MMP-9-mediated shedding was inconsequential in a longitudinal study performed pre- and post-anti-TNFα therapy for 6 weeks (Deyab G, et al. PLoS One 2021; 16:e0253247). Hence, pathogenicity of Syntenin-1 and SDC-1 was characterized in RA MΦs.

Myeloid cells exposed to Syntenin-1 display activated SRC, AKT, STAT1, NF-κB, and JNK signaling. While AKT, STAT3, and JNK signaling pathways are distinct to myeloid cells stimulated by Syntenin-1, activation of SRC, p38 MAPK, and NF-κB by Syntenin-1 is also required for human melanoma cell motility and invasion (Boukerche H, et al. Oncogene. 2010; 29:3054-66; and Boukerche H, et al. Cancer Res 2005; 65:10901-11). In Syntenin-1-differentiated RA MΦs or arthritic joints, the pronounced inflammatory landscape was developed by overexpression of IRF1/7/8/9 and IL-1p, IL-6, TNF, CCL2 or CCL5, CXCL2 over the pro-repair profile exhibited as IRF3/IRF4 and TGFβ. Moreover, the dominance of the inflammatory network in RA MΦs or arthritic joints fostered by Syntenin-1 was accompanied by robust Th1 cell differentiation and glycolytic hyperactivity. Particularly, RA CD14⁺CD86⁺GLUT1⁺MΦs rewired by Syntenin-1 generate their ATP mainly through glycolysis (% χglycoATP) over mitochondrial oxidative phosphorylation (% ←mitoATP) 15 which results in GLUT1. HK2, HIF1α, RAPTOR. LDHA escalation, and lactate secretion.

Distinct from SDC-1 blockade, IL-5R Ab or PDZ1i therapy was ineffective in the Syntenin-1-escalated inflammatory landscape in RA Mis. This is in part due to the lack of IL-5R expression in RA MΦs, despite the cell-type-specific interaction of Syntenin-1 with IL-5 and IL-5R in eosinophil differentiation (Beekman J M, et al. Blood. 2009; 114:3917-27) and mucosal IgA production in B cells (Moon B-gon, et al. J Immunol. 2004:172:6020-9). The inability of PDZli to nullify Syntenin-1-instigated RA MΦ reprogramming is also inconsistent with the involvement of PDZ1 in advancing IL-1β secretion from myeloid cells in breast cancer (Pradhan A K, et al. Proc Natl Acad Sci USA 2021; 118). Corroborating these findings, SDC-1 interaction with Syntenin-1 is distinctly facilitated through PDZ2 connection (Koroll M. et al. J Biol Chem 2001; 276:10646-54; and Kang B S. et al. Structure. 2003; 11:845-53).

Substantiating the data in RA pathology, SDC-1^{-/-} mice were resistant to arthritis developed by local Syntenin-1 expression through restriction of F4/80⁺iNOS⁺MΦs and CD3′ T cell infiltration. It was noted that the joint MΦs inflammatory (IL-6, IL-IlQ, TNF, CCL2, CCL5, CXCL2, IL-12, and IL-18) and metabolic (GLUT1, HIFα, RAPTOR/mTOR, and LDHA) landscapes were counteracted in SDC-1^{-/-} relative to WT mice induced with Syntenin-1-mediated arthritis. However, the oxidative profile was modestly attenuated by joint AMPK and PPARγ upregulation in SDC-1^{-/-} relative to WT animals ectopically expressing Syntenin-1. Extending these findings, CIA joint inflammation, metabolic activity, bone erosion and vascularization were attenuated in SDC-1^{-/-} compared with WT mice (Meyer A, et al. Cell Mol Immunol 2022; 19:1070-2).

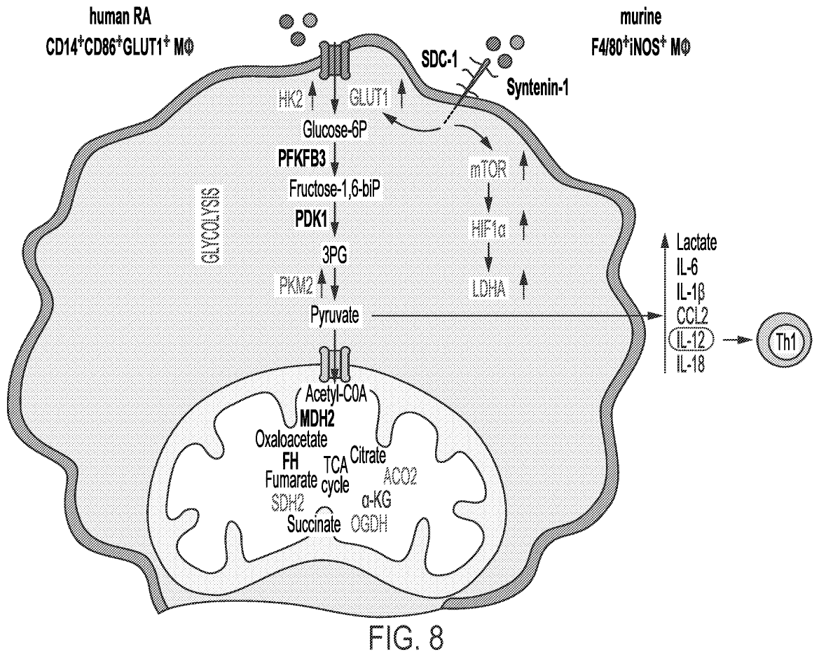

It was shown that T cell recruitment was amplified by Syntenin-1-mediated arthritis and was diminished in melanoma metastasis in Syntenin-1^{-/-} mice (Das S K, et al. Oncotarget 2016; 7:46848-61). Syntenin-1 was capable of differentiating RA PBMCs into Th1 and Th17 cells. In contrast, although Th1 cells were detected in Syntenin-1 arthritic mice via IL-12 and IL-18 induction, joint Th17 cells were unaffected in this process. IL-12 blockade diminished Th1 cell polarization amplified by syntenin-1 in RA PBMCs compared with T cells alone; further highlighting its significance in MD and T cell cross-regulation. Remarkably, RA CD14⁺CD86⁺GLUT1⁺MΦs, Th1, and Th17 cells reprogramming by Syntenin-1 were dysregulated by inhibition of mTOR signaling and glucose uptake but not HIF1α dysregulation. Two mTOR subunits, namely RAPTOR and RICTOR are involved in the rewiring of pro-inflammatory and pro-repair/regulatory MΦs and T cells, respectively (Covarrubias A J, et al. Semin Immunol 2015; 27:286-96; Cheng S-C, et al. Science 2014; 345:1250684; Kelly B and O'Neill L A J. Cell Res 2015; 25:771-84; Corcoran S E, et al. J Clin Invest 2016:126:3699-707; and Huang H, et al. Immunol Rev 2020:295:15-38). Classical MΦ polarization via mTOR has shown to be dependent on AKT, NF-κB, and JNK pathways (Covarrubias A J, et al. Semin Immunol 2015; 27:286-96), hence activation of these cascades in Syntenin-1-stimulated myeloid cells may be linked to mTOR/RAPTOR signaling. Similarly, in classical and Syntenin-1-differentiated MΦs the metabolic activity is reciprocally expanded via GLUT1, LDHA, and lactate which can be impaired by 2-DG therapy (Umar S, et al. Cell Mol Life Sci 2021; 78:7693-707). In CIA, SDC-1 deficiency can markedly suppress joint GLUT1 and mTOR hyperactivation observed in WT animals (Meyer A, et al. Cell Mol Immunol 2022; 19:1070-2). Others have shown that mTOR/RAPTOR activity plays an important role in Th17 transdifferentiation into a Th1-like subset (Karmaus P W F, et al. Nature 2019; 565:101-5). Previous studies also demonstrate that mTOR deficiency compromises Th1 and Th17 cell differentiation by restraining inflammatory monokines, IL-12, IL-6, and IL-1β (Umar S, et al. Cell Mol Life Sci 2021; 78:7693-707; and Covarrubias A J, et al. Semin Immunol 2015; 27:286-96). These results show that mTOR-potentiated glycolysis is accountable for Syntenin-1 cross-regulation of metabolic MΦs and Th1 cells (FIG. 8).

Recapitulating the connection between Syntenin-1/SDC-1 cascade and radiographic bone erosion, osteoclast formation in Syntenin-1 exposed RA cells and arthritic joints are cultivated through RANK. CTSK, and NFATc1 induction which is contingent on the SDC-1 function. In short, dysregulation of the Syntenin-1/SDC-1 signaling can provide a therapeutic strategy for RA patients who have a robust innate and adaptive activation and do not respond to current biologics.

### Example 2. Metabolic Reprogramming by Syntenin-1 Directs R A FLS and Endothelial Cell-Mediated Inflammation and Angiogenesis

A RA synovial fluid protein, Syntenin-1, and its receptor, Sydecan-1 (SDC-1), are colocalized on RA synovial tissue endothelial cells and fibroblast-like synoviocytes (FLS). Syntenin-1 exacerbates the inflammatory landscape of endothelial cells and RA FLS by upregulating transcription of IRF1/5/7/9, IL-1β, IL-6, and CCL2 through SDC-1 ligation and HIF1α or mTOR activation. Mechanistically, Syntenin-1 orchestrates RA FLS and endothelial cell invasion via SDC-1 and/or mTOR signaling. In Syntenin-1 reprogrammed endothelial cells, the dynamic expression of metabolic intermediates coincides with escalated glycolysis along with unchanged oxidative factors, AMPK, PGC-1a, citrate, and inactive oxidative phosphorylation. Conversely, RA FLS rewired by Syntenin-1 displayed a modest glycolytic-ATP accompanied by a robust mitochondrial-ATP capacity. The enriched mitochondrial-ATP detected in Syntenin-1 reprogrammed RA FLS was coupled with mitochondrial fusion and fission recapitulated by escalated Mitofusin-2 and DRP1 expression. The results described herein show that VEGFR1/2 and Notch1 networks are responsible for the crosstalk between Syntenin-1 rewired endothelial cells and RA FLS, which are also represented in RA explants. Similar to RA explants, morphological and transcriptome studies authenticated the importance of VEGFR1/2, Notch1, RAPTOR, and HIF1α pathways in Syntenin-1 arthritic mice and their obstruction in SDC-1 deficient animals. Consistently, dysregulation of SDC-1, mTOR, and HIF1α negated Syntenin-1 inflammatory phenotype in RA explants, while inhibition of HIF1α impaired synovial angiogenic imprint amplified by Syntenin-1. In conclusion, since the current therapies are ineffective on Syntenin-1 and SDC-1 expression in RA synovial tissue and blood, targeting this pathway and its interconnected metabolic intermediates can provide an alternative therapeutic strategy.

Melanoma differentiation-associated gene-9 (MDA) or Syntenin-1 is a cytosolic adaptor protein that can bind to the intracellular domain of Syndecan (SDC-1, surface heparan sulfate proteoglycan) through its PDZ2 domain activating the phosphorylation of FAK, Src, p38 MAPK, and AKT in melanoma and breast cancer cells (Boukerche H, et al. Proc Natl Acad Sci USA. 2008; 105:15914-15919; and Boukerche H, et al. Oncogene. 2010; 29:3054-3066). In parallel, Syntenin-1 has other binding partners, including CD63, Merlin, and IL-5R, that bind to its PDZ1 domain (Kang B S, et al. Structure. 2003; 11:459-468; and Latysheva N, et al. Mol Cell Biol. 2006; 26:7707-7718). The PDZ1 domain exhibits weak binding to its target proteins, conversely the Syntenin-1-interacting protein, SDC-1 has a stronger binding capacity to the PDZ2 domain (Kang B S, et al. Structure. 2003: 11:459-468; and Grembecka J, et al. Biochemistry. 2006; 45:3674-3683).

Overexpression of Syntenin-1 in lung cancer tissue and sera was linked to poor prognosis (Luo P, et al. BMC Cancer. 2020: 20:159), and Syntenin-1 KO mice displayed delayed tumor initiation and mitigated lung metastasis (Das S K, et al. Oncotarget. 2016; 7:46848-46861). Consistently, elevated SDC-1 sera in lung (Holli K, et al. J Clin Oncol. 2009; 27:927-932) or liver (Nault J C, et al. Cancer Epidemiol Biomarkers Prev. 2013; 22:1343-1352) cancer patients and its potentiated protein expression in the stroma and tumor cells in gastric and pancreatic cancer (Wiksten J P, et al. Int J Cancer. 2001; 95:1-6) correlated with a high risk of recurrence and metastatic potential. In contrast, others report that SDC-1-deficient mice exhibit advanced tumor growth in colitis-induced colon carcinoma because of escalated IL-6 production and STAT3 signaling (Binder Gallimidi A, et al. PLoS One. 2017: 12:e0174343). Similarly, SDC-1 KO mice subjected to imiquimod-induced psoriasis illustrated accentuated skin inflammation compared to the wild-type (WT) mice in part due to the expansion of Ty617 cells (Jaiswal A K, et al. J Immunol. 2018; 201:1651-1661). Moreover, SDC-1 KO mice displayed altered metabolism due to glucose intolerance and insulin resistance (Jaiswal A K, et al. World J Diabetes. 2020; 11:126-136).

Syntenin-1 is enriched in rheumatoid arthritis (RA) relative to osteoarthritis (OA) synovial fluid (SF) (Meyer A, et al. Ann Rheum Dis. 2023). The expression of Syntenin-1 and SDC-1 is amplified on RA synovial tissue (ST) lining, sublining, and blood vessels compared to normal counterparts, where the ligand and the receptor colocalize (Meyer A, et al. Ann Rheum Dis. 2023; and Van Raemdonck K, et al. Arthritis Rheumatol. 2021). RNA-seq analysis revealed that Syntenin-1 and SDC-1 transcriptomes were linked to the number of CD68⁺macrophages (MΦs) in RA STs (Meyer A, et al. Ann Rheum Dis. 2023; and Lewis M J, et al. Cell reports. 2019; 28:2455-2470 e2455). Interestingly, Syntenin-1 and SDC-1 expression are mutually elevated by LPS/IFNγ stimulation in RA monocyte-differentiated MΦs. The Syntenin-1 transcriptome in RA blood is connected to CCP and bone erosion (Meyer A, et al. Ann Rheum Dis. 2023; and Lewis M J, et al. Cell reports. 2019; 28:2455-2470 e2455). Accordingly, SDC-1 expression in RA synovial tissue is implicated in ultrasound (US) ST thickness and radiographic bone erosion (Meyer A. et al. Ann Rheum Dis. 2023; and Lewis M J, et al. Cell reports. 2019; 28:2455-2470 e2455). Distinct from these findings, others have shown that SDC-1 transcription levels were downregulated at the erosive site relative to intact osteoclast cartilage (Barre P E, et al. Osteoarthritis Cartilage. 2000; 8:34-43). Recapitulating the association of blood Syntenin-1 and synovial SDC-1 with total Sharp x-ray score, RA precursor cells exposed to Syntenin-1 were reconfigured into mature osteoclasts via transcriptional upregulation of RANK, CTSK, and NFATc1 (Meyer A, et al. Ann Rheum Dis. 2023; and Lewis M J, et al. Cell reports. 2019; 28:2455-2470 e2455).

Earlier studies have unmasked that the pathogenic effect of Syntenin-1 is advanced by reprogramming naive cells into metabolic RA CD14⁺CD86⁺GLUT1⁺MΦs that can cross-regulate Th1 cells in part through IL-12 and IL-18 induction (Meyer A, et al. Ann Rheum Dis. 2023). Moreover, collagen-induced arthritis (CIA) was mitigated in SDC1 KO mice due to constrained joint F480⁺iNOS⁺MΦs frequency and diminished IL-6 and IL-1β transcription compared to wild-type mice (Meyer A, et al. Cell Mol Immunol. 2022; 19:1070-1072). Nevertheless, the molecular mechanism and malfunctioning metabolic machinery of Syntenin-1 and SDC-1 are undefined in endothelial cells, RA fibroblast-like synoviocytes (FLS), and RA explants.

Described herein is the finding that Syntenin-1 and SDC-1 are colocalized on RA endothelial cells and FLS and cross-link the arthritogenicity of these cells by influencing their inflammatory, angiogenic, and metabolic landscapes. Endothelial cells exposed to Syntenin-1 exhibit an inflammatory and proangiogenic reconfiguration along with escalated glycolysis through SDC-1, RAPTOR, and HIF1α signaling. RA FLS reprogrammed by Syntenin-1 display an inflammatory and oxidative stress phenotype, related to SDC-1 and HIF1α activation that coincides with mitochondrial dysregulation via Mitofusin-2 and DRP1 induction. Nonetheless, the glycolytic profile of RA FLS reprogrammed by Syntenin-1 is restricted to RAPTOR which can also modulate its migration. The Syntenin-1-induced arthritis model exemplifies Syntenin-1-activated RA explants by highlighting the significance of inflammatory and proangiogenic networks and their connection to SDC-1, RAPTOR, and HIF1α pathways. Importantly, the results show that the VEGFR1/2 and Notch1 axes play an important role in Syntenin-1-induced interplay between endothelial cells and RA FLS which is represented in RA explants. Notably, in RA explants, inhibition of SDC-1, mTOR, and HIF1α, dysregulated the Syntenin-1-enhanced inflammatory remodeling, while HIF1αi was also responsible for disrupting the angiogenic profile.

Materials and methods. Cells. FLS from fresh RA ST were isolated by mincing and digestion in a solution of dispase, collagenase, and DNase. Cells were used between passages 3 and 9 (Pickens S R, et al. Characterization of CCL19 and CCL21 in rheumatoid arthritis. Arthritis Rheum. 2011; 63:914-922; Pickens S R, et al. Arthritis Rheum. 2011; 63:2884-2893; Chamberlain N D, et al. J Immunol. 2012; 189:475-483; and Elshabrawy H A. et al. Angiogenesis. 2018; 21:215-228). Human umbilical vein endothelial cells (HUVECs) were purchased from Lonza and used between passages 3 and 9 (Elshabrawy H A, et al. Angiogenesis. 2018; 21:215-228; Chen Z, et al. Ann Rheum Dis. 2015; 74:1898-1906; and Kim S J, et al. Arthritis Rheum. 2013; 65:2024-2036). HUVECs were used as surrogates for RA endothelial cells as an adequate number of cells could not be isolated from RA STs.

Syntenin-1 stimulation and inhibition. HUVECs or RA FLS were either untreated (PBS) or treated with Syntenin-1 (1000 ng/ml, NKMAX Co.) for 6 h to 48 h. For blocking specific mechanism of action, cells were starved overnight in the presence of 2-deoxy-D-glucose (2-DG; 5 mM. Sigma-Aldrich, St. Louis, USA), hypoxia-inducible factor 1a inhibitor (HIF1αi; 2 μM, Calbiochem), mTOR inhibitor (mTORi; 1 μM; Everolimus, Sigma-Aldrich), cMYCi (50 μM, Sigma-Aldrich), human IL-5R antibody (IL5Ra; 2 μg/ml, R&D Systems), PDZ1 domain inhibitor peptide (PDZ1; 10 μM, Tocris Bioscience), or SDC-1 antibody (SDCab; 1:100, Diaclone) following Syntenin-1 (1000 ng/ml. NKMAX Co.) stimulation for 6 h or 24 h. Cells were subsequently harvested in TRIzol reagent (Life Technologies) or RIPA buffer (Cell Signaling Technology) for mRNA quantification and western blot analysis; conditioned media was collected for ELISA.

RA FLS and HUVEC scratch assay. A scratch was created in the middle of the wells that contained confluent HUVECs or RA FLS. Thereafter, cells were either untreated (PBS) or treated with Syntenin-1 (1000 ng/ml), or 10% FBS and bFGF (100 ng/ml) as a positive control for 24 h. In parallel, cells were treated with SDC1-Ab (1:100), IL-5R Ab (2 μg/ml), PDZli (10 μM), mTORi (IVM), or HIF1αi (2 μM) for 24 h. In the scratch assay experiments, cells were fixed with 10% formalin for 1 h at 37° C. and were subsequently stained with 0.05% crystal violet for 1 h before imaging. The number of cells in the scratch area was counted and compared to the untreated control.

Animal studies. Wild-type C57BL/6 mice (>8 weeks old; Jackson Laboratory, Bar Harbor, Maine, USA) were bred in-house. SDC-1^{-/-} mice (Alexander C M, et al. Nat Genet. 2000; 25:329-332). Animals were housed in sterile static micro isolator cages on autoclaved corncob bedding with water bottles in a specific-pathogen-free (SPF) facility. Animal food is irradiated, and water is autoclaved. Both food and water are provided ad libitum. The standard photoperiod for rodent rooms is 14 hours of light and 10 hours of darkness. Animals were provided with autoclaved nesting materials. Cages are changed at least weekly in either a biosafety cabinet or a HEPA-filtered animal transfer station. Eight- to twelve-week-old WT and SDC-1^{-/-} mice were injected intra-articularly with adenovirus (ad)-ctrl or ad-Syntenin-1 (3×10¹⁰ viral particles/ankle, Welgen) on days 0, 7, and 14. Joint circumference was assessed by a caliper and mice were sacrificed on day 15. Ankles were harvested and used for further analysis.

RheumatoidArthritis Explants. RA ST (30 mg) was cut into small pieces to allow proper access to stimuli and were starved o/n in 0% FBS RPMI with or without SDC1-Ab (1:100), mTOR1i (1 μM), and HIF1αi (2 M). RA STs were stimulated with 5000 ng/ml Syntenin-1 (1000 ng/ml) for 6-8 h. Tissues were harvested for transcriptome analysis and supernatants were used for protein quantification by ELISA.

RNASeq transcriptome and Single-cell RNAseq Transcriptome Analysis. The web interface peac.hpc.qmul.ac.uk/ developed by Lewis et al. (Lewis M J, et al. Cell reports. 2019; 28:2455-2470 e2455) was used to correlate the expression of Syntenin-1 and SDC-1 in blood and synovial biopsies from individuals with early rheumatoid arthritis in the Pathobiology of Early Arthritis Cohort (PEAC) study. The DAS-based European League Against Rheumatism (EULAR) response criteria were generated to quantify individual responses in clinical trials. The EULAR response criteria classify individual patients as non-(ΔDAS28≤0.6), moderate (ΔDAS28≤1.2 & >0.6), or good responders (ΔDAS28>1.2). Gene transcript expression levels are expressed as VST (variance stabilizing transformation) transformed read counts using the Bioconductor package DESeq2. Synovial histology was scored using a semiquantitative grading from 0-4 (Humby F, et al. PLoS Med. 2009: 6:e1). The raw RNA-Seq data have been deposited at ArrayExpress accession E-MTAB-6141.

The single-cell RNA sequencing data from Wei et al. (Wei K. et al. Nature. 2020; 582:259-264) was accessed from the Broad Institute Single Cell portal at the following URL: singlecell.broadinstitute.org/single_cell/study/SCP469/synovial-fibroblast-positional-identity-controlled-bv-inductive-notch-signaling-underlies-pathologic-damage-in-inflammatorv-arthritis. A cohort of RA patients that fulfilled the ACR 2010 Rheumatoid Arthritis classification criteria were included. Synovial tissue samples were acquired when the patients underwent either joint replacement or synovectomy procedures.

The RNAseq dataset GSE198520 was accessed (ncbi.nlm.nih.gov/geo/geo2r/?acc=GSE198520/) deposited by Wang et al. (Wang J, et al. Arthritis Rheumatol. 2022; 74:1916-1927) to evaluate the expression of SDCBP and SDC-1 in RA synovium biopsied from 27 RA patients 12 weeks after treatment with anti-TNF (Certolizumab). Patients in this cohort fulfilled the 2010 ACR/EULAR RA Classification Criteria and were enrolled at the Centre for Experimental Medicine and Rheumatology, Barts and The London School of Medicine, Queen Mary University of London, UK. RA patients exhibited clinically defined synovitis and fit the criteria for UK NICE guidelines (failure of at least 2 csDMARDs and DAS28>=5.1) to start anti-TNF therapy. Following enrollment, patients underwent minimally invasive U.S.-guided synovial biopsy of the most inflamed joint (ultrasound synovial thickening score>=2). The patient data were grouped and displayed based on whether the patients were considered non-responders, moderate responders, or good responders to anti-TNF therapy. Response to therapy was evaluated using ACR/EULAR DAS28 response criteria defined as good response (ΔDAS [DAS28 at baseline—DAS28 at 12 weeks after treatment]>1.2 with DAS28 at 12 weeks≤3.2), moderate response (DAS28 change>1.2 with DAS28 at 12 weeks>3.2, or DAS28 change 0.6-1.2 with DAS28 at 12 weeks≤5.1), or nonresponse (DAS28≤0.6, or DAS28 change 0.6-1.2 with DAS28 at 12 weeks>5.1) (van Riel P L and Renskers L. Clin Exp Rheumatol. 2016; 34:S40-S44). Data were further separated based on whether the patients were non-responders or responders to anti-TNF (Certolizumab) therapy.

The web interface (r4ra.hpc.qmul.ac.uk/) developed by Rivellese et al. (Rivellese F, et al. Nat Med. 2022; 28:1256-1268) was used to evaluate the expression of SDCBP and SDC-1 in synovial tissue from RA patients that were treated with rituximab or tocilizumab. A cohort of 164 patients aged 18 years or over who fulfilled the 2010 American College of Rheumatology/European Alliance of Associations for Rheumatology (EULAR) classification for RA and were eligible for treatment with rituximab therapy according to UK NICE guidelines (patients who failed or were intolerant to csDMARD therapy and at least one biologic therapy) were included in the trial. Initially, a synovial biopsy was taken of a clinically active joint at the beginning of the trial. Patients were then randomized to rituximab or tocilizumab treatment administered as either two 1,000-mg intravenous rituximab infusions 2 weeks apart or intravenous tocilizumab at a dose of 8 mg/kg at 4-week intervals. The patient data were grouped based on response to therapy using the ACR/EULAR DAS28 C reactive protein (CRP) response criteria as described herein.

Quantitative Real-Time PCR. According to the manufacturer's instructions. RNA was isolated using a TRIzol reagent (Life Technologies). Transcription to cDNA and subsequent quantitative real-time PCR analysis was performed using the High-Capacity cDNA Reverse Transcription Kit (Applied Biosystems) and TaqMan Gene Expression Master Mix (Applied Biosvstems). Predesigned IDT primers or TaqMan gene expression assays were used (Tables 1 and 2). Data are presented as fold change (2^{-ΔΔct}) normalized to the housekeeping gene (actin) and compared to the control. Data were acquired with the QuantStudio5 (Applied Biosystems) qRT-PCR device.

TABLE 1
Predesigned IDT primers and TaqMan gene expression assays
used to quantify mRNA expression of human samples.

| Gene | | | | |
|---|---|---|---|---|
| Name | Assay ID | Company | Ref Seq # | Assay Configuration |
| ACTB | Hs.PT.56a.3404650.g | IDT | NM_001101 | Std, FAM/ZEN/IBFQ, |
| | | | | P:P 2 |
| AMPK | Hs.PT.58.700639 | IDT | NM_006251 | Std, FAM/ZEN/IBFQ, |
| | | | | P:P 2 |
| ATG5 | Hs.PT.58.2898629 | IDT | NM_004849 | Std, FAM/ZEN/IBFQ, |
| | | | | P:P 2 |
| ATG7 | Hs.PT.58.4019800 | IDT | NM_004849 | Std, FAM/ZEN/IBFQ, |
| | | | | P:P 2 |
| BECLIN1 | Hs.PT.58.504143 | IDT | NM_003766 | Std, FAM/ZEN/IBFQ, |
| | | | | P:P 2 |
| bFGF | Hs.PT.58.24613308 | IDT | NM_002006 | Std, FAM/ZEN/IBFQ, |
| | | | | P:P 2 |
| CCL2 | Hs.PT.58.45467977 | IDT | NM_002982 | Std, FAM/ZEN/IBFQ, |
| | | | | P:P 2 |
| CCL5 | Hs.PT.58.1724551 | IDT | NM_002985 | Std, FAM/ZEN/IBFQ, |
| | | | | P:P 2 |
| cMYC | Hs.PT.58.26770695 | IDT | NM_002467 | Std, FAM/ZEN/IBFQ, |
| | | | | P:P 2 |
| CXCL1 | Hs.PT.58.39039397 | IDT | NM_046035 | Std, FAM/ZEN/IBFQ, |
| | | | | P:P 2 |
| CXCL5 | Hs.PT.58.41058007.g | IDT | NM_002994 | Std, FAM/ZEN/IBFQ, |
| | | | | P:P 2 |
| CXCR5 | Hs.PT.56a.1692541 | IDT | NM_001716 | Std, FAM/ZEN/IBFQ, |
| | | | | P:P 2 |
| DLL1 | Hs.PT.58.41063402 | IDT | NM_005618 | Std, FAM/ZEN/IBFQ, |
| | | | | P:P 2 |
| DLL4 | Hs.PT.58.3416363 | IDT | NM_019074 | Std, FAM/ZEN/IBFQ, |
| | | | | P:P 2 |
| GLUT1 | Hs.PT.58.25872862 | IDT | NM_006516 | Std, FAM/ZEN/IBFQ, |
| | | | | P:P 2 |
| GPR81 | Hs.PT.58.27202899.g | IDT | NM_032554 | Std, FAM/ZEN/IBFQ, |
| | | | | P:P 2 |
| HIF1A | Hs.PT.58.534274 | IDT | NM_001243084 | Std, FAM/ZEN/IBFQ, |
| | | | | P:P 2 |
| HK2 | Hs.PT.58.4863675 | IDT | NM_000189 | Std, FAM/ZEN/IBFQ, |
| | | | | P:P 2 |
| IFNA | Hs.PT.58.46311748.g | IDT | NM_000605 | Std, FAM/ZEN/IBFQ, |
| | | | | P:P 2 |
| IFNB | Hs.PT.58.39481063.g | IDT | NM_002176 | Std, FAM/ZEN/IBFQ, |
| | | | | P:P 2 |
| IFNG | Hs.PT.58.3781960 | IDT | NM_000619 | Std, FAM/ZEN/IBFQ, |
| | | | | P:P 2 |
| IL1 | Hs.PT.58.40913627 | IDT | NM_000575 | Std, FAM/ZEN/IBFQ, |
| | | | | P:P 2 |
| IL10 | Hs.PT.58.2807216 | IDT | NM_000572 | Std, FAM/ZEN/IBFQ, |
| | | | | P:P 2 |
| IL18 | Hs.PT.58.25675872 | IDT | NM_001243211 | Std, FAM/ZEN/IBFQ, |
| | | | | P:P 2 |
| IL18R | Hs.PT.58.2971462 | IDT | NM_003855 | Std, FAM/ZEN/IBFQ, |
| | | | | P:P 2 |
| IL6 | Hs.PT.58.40226675 | IDT | NM_000600 | Std, FAM/ZEN/IBFQ, |
| | | | | P:P 2 |
| IL8 | Hs.PT.58.39926886.g | IDT | NM_000584 | Std, FAM/ZEN/IBFQ, |
| | | | | P:P 2 |
| IRF1 | Hs.PT.58.26847423 | IDT | NM_002198 | Std, FAM/ZEN/IBFQ, |
| | | | | P:P 2 |
| IRF3 | Hs.PT.58.19791480 | IDT | NM_001197124 | Std, FAM/ZEN/IBFQ, |
| | | | | P:P 2 |
| IRF4 | Hs.PT.58.2073651 | IDT | NM_001195286 | Std, FAM/ZEN/IBFQ, |
| | | | | P:P 2 |
| IRF5 | Hs.PT.58.20692129 | IDT | NM_001242452 | Std, FAM/ZEN/IBFQ, |
| | | | | P:P 2 |
| IRF7 | Hs.PT.58.24613215.g | IDT | NM_001572 | Std, FAM/ZEN/IBFQ, |
| | | | | P:P 2 |
| IRF8 | Hs.PT.58.455867 | IDT | NM_002163 | Std, FAM/ZEN/IBFQ, |
| | | | | P:P 2 |
| IRF9 | Hs.PT.58.3264634 | IDT | NM_006084 | Std, FAM/ZEN/IBFQ, |
| | | | | P:P 2 |
| JAG1 | Hs.PT.56a.4972610 | IDT | NM_000214 | Std, FAM/ZEN/IBFQ, |
| | | | | P:P 2 |
| JAG2 | Hs.PT.58.3493191 | IDT | NM_145159 | Std, FAM/ZEN/IBFQ, |
| | | | | P:P 2 |
| LAMP1 | Hs.PT.58.27192505 | IDT | NM_005561 | Std, FAM/ZEN/IBFQ, |
| | | | | P:P 2 |
| LDHA | Hs.PT.58.22929122 | IDT | NM_001165416 | Std, FAM/ZEN/IBFQ, |
| | | | | P:P 2 |
| LDHB | Hs.PT.58.1489513 | IDT | NM_002300 | Std, FAM/ZEN/IBFQ, |
| | | | | P:P 2 |
| MCT1 | Hs.PT.58.39162089 | IDT | NM_001166496 | Std, FAM/ZEN/IBFQ, |
| | | | | P:P 2 |
| MCT4 | Hs.PT.58.24353244 | IDT | NM_001201546 | Std, FAM/ZEN/IBFQ, |
| | | | | P:P 2 |
| NOTCH1 | Hs.PT.58.23074795 | IDT | NM_017617 | Std, FAM/ZEN/IBFQ, |
| | | | | P:P 2 |
| P62 | Hs.PT.58.2641907 | IDT | NM_001007225 | Std, FAM/ZEN/IBFQ, |
| | | | | P:P 2 |
| PFK2 | Hs.PT.58.25935895 | IDT | NM_001145443 | Std, FAM/ZEN/IBFQ, |
| | | | | P:P 2 |
| PGC1α | Hs.PT.58.14965839 | IDT | NM_013261 | Std, FAM/ZEN/IBFQ, |
| | | | | P:P 2 |
| PKM2 | Hs.PT.58.14798218 | IDT | NM_001206796 | Std, FAM/ZEN/IBFQ, |
| | | | | P:P 2 |
| RAPTOR | Hs.PT.58.26749458 | IDT | NM_020761 | Std, FAM/ZEN/IBFQ, |
| | | | | P:P 2 |
| SIRT1 | Hs.PT.58.38627809 | IDT | NM_001142498 | Std, FAM/ZEN/IBFQ, |
| | | | | P:P 2 |
| SIRT3 | Hs.PT.58.40981592.g | IDT | NM_012239 | Std, FAM/ZEN/IBFQ, |
| | | | | P:P 2 |
| SIRT5 | Hs.PT.58.26285449.g | IDT | NM_012241 | Std, FAM/ZEN/IBFQ, |
| | | | | P:P 2 |
| TGFB | Hs.PT.58.39813975 | IDT | NM_000660 | Std, FAM/ZEN/IBFQ, |
| | | | | P:P 2 |
| TLR2 | HS01872448_s1 | TaqMan, | NM_003264 | Std, FAM/ZEN/IBFQ, |
| | | Applied | | P:P 2 |
| | | Biosystems | | |
| TLR4 | HS00152939_m1 | TaqMan, | NM_003266 | Std, FAM/ZEN/IBFQ, |
| | | Applied | | P:P 2 |
| | | Biosystems | | |
| TLR5 | HS00152825_m1 | TaqMan, | NM_003268 | Std, FAM/ZEN/IBFQ, |
| | | Applied | | P:P 2 |
| | | Biosystems | | |
| TLR7 | Hs.PT.58.38778009 | IDT | NM_016562 | Std, FAM/ZEN/IBFQ, |
| | | | | P:P 2 |
| TLR8 | HS00152981_m1 | TaqMan, | NM_138636 | Std, FAM/ZEN/IBFQ, |
| | | Applied | | P:P 2 |
| | | Biosystems | | |
| TNFA | HS01113624_g1 | TaqMan, | NM_000594 | Std, FAM/ZEN/IBFQ, |
| | | Applied | | P:P 2 |
| | | Biosystems | | |
| VEGF | Hs.PT.58.1149801 | IDT | NM_003376 | Std, FAM/ZEN/IBFQ, |
| | | | | P:P 2 |
| VEGFR1 | Hs.PT.58.40906831 | IDT | NM_001160030 | Std, FAM/ZEN/IBFQ, |
| | | | | P:P 2 |
| VEGFR2 | Hs.PT.58.3285240 | IDT | NM_002253 | Std, FAM/ZEN/IBFQ, |
| | | | | P:P 2 |

TABLE 2
Predesigned IDT primers and TaqMan gene expression assays
used to quantify mRNA expression of murine samples.

| Gene | | | | |
|---|---|---|---|---|
| name | Assay ID | Company | Ref Seq # | Assay Configuration |
| HIF1A | Mm.PT.58.11211292 | IDT | NM_010431 | Std, FAM/ZEN/IBFQ, |
| | | | | P:P 2 |
| NOTCH1 | Mm.PT.58.28794468 | IDT | NM_008714 | Std, FAM/ZEN/IBFQ, |
| | | | | P:P 2 |
| RAPTOR | Mm.PT.58.30504526 | IDT | NM_028898 | Std, FAM/ZEN/IBFQ, |
| | | | | P:P 2 |
| VEGFR1 | Mm.PT.58.43852013 | IDT | NM_010228 | Std, FAM/ZEN/IBFQ, |
| | | | | P:P 2 |

Technology) supplemented with protease and phosphatase inhibitors (Roche laboratories) and protein concentration was assessed with the Pierce BCA Protein Assay Kit (ThermoFisher Scientific) following the manufacturer's instructions. Lysates were run on 10% polyacrylamide gels. Blotting was performed with the Trans-Blot Turbo Transfer System (Bio-Rad Laboratories). Samples were subsequently probed for pSrc, pAKT, pSTATI, pSTAT3, p-p38, pERK, pJNK, IxBa, GLUT1, HK2, PFK2, cMYC, HIF1α, LDHA, AMPK, Mitofusin-2 and DRP1 (1:1000, Cell Signaling Technology), RAPTOR, Notch1 and mTOR (1:1000, Santa Cruz), β-actin (1:3000, Santa Cruz), and anti-rabbit IgG HRP-linked or anti-mouse IgG HRP-linked (1:2500, both Cell Signaling Technology) (Table 3). Detection was performed using the iBright 1500 (Invitrogen by ThermoFisher Scientific).

TABLE 3
Complete list of antibodies used for protein
detection by western blot analysis.

| | | | | Species |
|---|---|---|---|---|
| Antibody | Company | Catalog # | Host | reactivity |
| AMPK | Cell Signaling | #2532 | rabbit | human, |
| | Technology | | | mouse |
| Anti-mTOR | Santa Cruz | sc-517464 | mouse | human, |
| Antibody | | | | mouse |
| Anti-β-actin | Santa Cruz | sc-47778 | mouse | human, |
| Antibody | | | | mouse |
| cMYC | Cell Signaling | #5605 | rabbit | human, |
| | Technology | | | mouse |
| DRP1 | Cell Signaling | #5391 | rabbit | human, |
| | Technology | | | mouse |
| Glut1 (D3J3A) | Cell Signaling | #12939 | rabbit | human, |
| Antibody | Technology | | | mouse |
| Hexokinase II | Cell Signaling | #2867 | rabbit | human, |
| (C64G5) Antibody | Technology | | | mouse |
| HIF1a | Santa Cruz | sc-53546 | mouse | human, |
| | | | | mouse |
| IκBα (L35A5) | Cell Signaling | #4814 | mouse | human, |
| Antibody | Technology | | | mouse |
| LDHA/LDHC | Cell Signaling | #3558 | rabbit | human, |
| (C28H7) Antibody | Technology | | | mouse |
| Mitofusin2 | Cell Signaling | #9482 | rabbit | human, |
| | Technology | | | mouse |
| PFKFB3/PFK2 | Cell Signaling | #13123 | rabbit | human, |
| (D7H4Q) Antibody | Technology | | | mouse |
| Phospho-Akt | Cell Signaling | #4060 | rabbit | human, |
| (Ser473) (D9E) XP | Technology | | | mouse |
| Antibody | | | | |
| Phospho-p38 | Cell Signaling | #9211 | rabbit | human, |
| MAPK | Technology | | | mouse |
| (Thr180/Tyr182) | | | | |
| Antibody | | | | |
| Phospho-p44/42 | Cell Signaling | #9101 | rabbit | human, |
| MAPK (Erk1/2) | Technology | | | mouse |
| (Thr202/Tyr204) | | | | |
| Antibody | | | | |
| Phospho- | Cell Signaling | #9251 | rabbit | human, |
| SAPK/JNK | Technology | | | mouse |
| (Thr183/Tyr185) | | | | |
| Antibody | | | | |
| Phospho-Src | Cell Signaling | #2105 | rabbit | human, |
| (Tyr527) Antibody | Technology | | | mouse |
| Phospho-Stat1 | Cell Signaling | #7649 | rabbit | human, |
| (Tyr701) (D4A7) | Technology | | | mouse |
| Antibody | | | | |
| Phospho-Stat3 | Cell Signaling | #9131 | rabbit | human, |
| (Tyr705) Antibody | Technology | | | mouse |
| RAPTOR | Santa Cruz | sc-81537 | mouse | human, |
| | | | | mouse |
| MFN2 | Cell Signaling | #9482 | rabbit | human, |
| | Technology | | | mouse |

Seahorse ATP Rate and Glycolysis Stress Test Kits. Extracellular acidification rate (ECAR) and oxygen consumption rate (OCR) were measured using the Seahorse XF Glycolysis Stress Test kit (Agilent Technologies) according to the manufacturer's instructions. HUVECs (5×10⁴ cells/well) were cultured for 2 days before the assay. Syntenin-1 (1 μg/ml) and PBS were injected during the experiment. Glycolysis and glycolytic capacity were calculated by the following equations, respectively: glycolysis=(Maximum rate measurement before Oligomycin injection)−(Last rate measurement before Glucose injection) and glycolytic capacity=(Maximum rate measurement after Oligomycin injection)−(Last rate measurement before Glucose injection). ATP production was calculated by the following equation: ATP production=(Last rate measurement before Oligomycin injection)−(Minimum rate measurement after Oligomycin injection).

Glycolytic ATP production (glycolysis) and mitochondrial ATP production (oxidative phosphorylation) were measured using the Seahorse XF ATP Rate Test kit (Agilent Technologies), according to the manufacturer's instructions. RA FLS (2×10⁵ cells/well) was cultured for 1 day and Syntenin-1 (1000 ng/ml) and PBS were injected during the experiment. Percent glycolysis increase and % oxidative phosphorylation decrease were calculated by the following equation:

$$\% Glycolysis ( INDUCED ) - \% Glycolysis ( BASAL ) = \% glycolysis increase .$$

Metabolite quantification. The concentration of the metabolites including pyruvate, lactate, citrate, and succinate was measured in conditioned media using the colorimetric assay kit (Sigma-Aldrich, St. Louis, USA) following the manufacturer's instructions.

Enzyme-Linked-Immunosorbent-Assay (ELISA). Human TNF-α, IL-1β, CCL5, IL-10, TGFβ, IL-8, and IL-12 protein levels were quantified by ELISA according to the manufacturer's instructions (R&D Systems, Minneapolis, MN).

Immunohistochemistry. RA ST formalin-fixed, paraffin-embedded samples were sectioned and stained for colocalization of Syntenin-1 (1:125), SDC-1 (1:500), VEGFR2 (1:25), Notch1 (1:50), RAPTOR (1:50), HIF1α (1:50), Mitofusin-2 (1:200), and DRP1 (1:200) on VWF⁺ endothelial cells (1:1000) and Vimentin⁺RA FLS (1:1000). Moreover, fluorescence secondary anti-rabbit (1:200) and anti-mouse (1:200) were utilized to visualize staining. Formalin-fixed mouse ankles were decalcified and paraffin-embedded. Slides were deparaffinized in xylene, and antigen retrieval was achieved (Van Raemdonck K, et al. Cell Mol Life Sci. 2020; 77:1387-1399). Mouse ankle sections were stained for Vimentin (1:1000). VWF (1:1000), VEGF2 (1:25), Notch1 (1:50), MFN2 (1:200), DRP1 (1:200) (Tables 4 and 5) staining and were scored on a scale of 0-5 in a blinded manner (0=normal appearance, 1=minimal changes, 2=mixed appearance, 3=moderate changes, 4=marked changes, and 5=severe changes) (Umar S, et al. Cell Mol Immunol. 2021; 18:2199-2210) at ×100 magnification. HUVECs were cultured on glass coverslips. Cells were treated with 1000 ng/mL Syntenin-1 for 18 h. Cells were fixed with 3.7% paraformaldehyde for 10 min, washed, then permeabilized with 0.1% saponin. Cells were stained with VEGFR2 (1:25) in PBS with 10% NDS and 0.01% sodium azide for 1 h at RT. Cells were washed then incubated with DAPI (1:1000) and FITC-fluorescently labeled secondary anti-mouse (1:300) Ab for 20 min. Cells were then washed and mounted on slides for imaging. Mean fluorescence intensity per cell was quantified using NIS-Elements Basic Research software.

TABLE 4
Primary antibodies used for protein
detection by Immunohistochemistry.

| | | | | Species |
|---|---|---|---|---|
| | | | | reac- |
| Antibody | Company | Catalog # | Host | tivity |
| Anti-SDCBP | aviva systems | ARP44535_T100 | rabbit | human, |
| Antibody | biology | | | mouse |
| Anti-Syndecan-1 | abcam | ab128936 | rabbit | human, |
| Antibody | | | | mouse |
| NOTCH1 | Santa Cruz | sc-376403 | mouse | human, |
| | | | | mouse |
| VECTASHIELD | Vector | #H-1800 | N/A | N/A |
| Vibrance | Laboratories | | | |
| Antifade | | | | |
| Mounting | | | | |
| Medium with | | | | |
| DAPI | | | | |
| VEGFR2 | R&D Systems | MAB3571-SP | mouse | human, |
| | | | | mouse |
| Vimentin | Lab Vision | RM-9120-R1 | rabbit | human, |
| | | (SP20) | | mouse |
| VWF | Dako | GA527 | rabbit | human, |
| | | GA52761-2 | | mouse |
| MFN2 | Cell Signaling | #9482 | rabbit | human, |
| | Technology | | | mouse |
| DRP1 | Cell Signaling | #5391 | rabbit | human, |
| | Technology | | | mouse |

TABLE 5
Fluorescent secondary antibodies used for protein
detection by Immunofluorescence staining.

| | | | | Species |
|---|---|---|---|---|
| | | | | reac- |
| Antibody | Company | Catalog # | Host | tivity |
| Alexa Fluor 488 | Jackson | #711-546-152 | donkey | rabbit |
| AffiniPure | ImmunoResearch | | | |
| F(ab′)₂ | | | | |
| Fragment | | | | |
| (green) | | | | |
| Alexa Fluor 488 | Jackson | #715-546-150 | donkey | mouse |
| AffiniPure | ImmunoResearch | | | |
| F(ab′)₂ | | | | |
| Fragment | | | | |
| (green) | | | | |
| Alexa Fluor ® | Jackson | #711-586-152 | donkey | rabbit |
| 594 AffiniPure | ImmunoResearch | | | |
| F(ab′)₂ | | | | |
| Fragment (red) | | | | |

Statistical Analysis. For comparison among multiple groups, one-way ANOVA followed by Tukey's multiple comparison tests was employed, using Graph Pad Prism9 software. The data were also analyzed using a two-tailed Student's t-test or Mann-Whitney test for paired or unpaired comparisons between two groups. When comparing RNA-Seq data against continuous or ordinal variables, the Spearman rank correlation test was used, and Spearman rho and p-values are shown. p<0.05 was considered statistically significant.

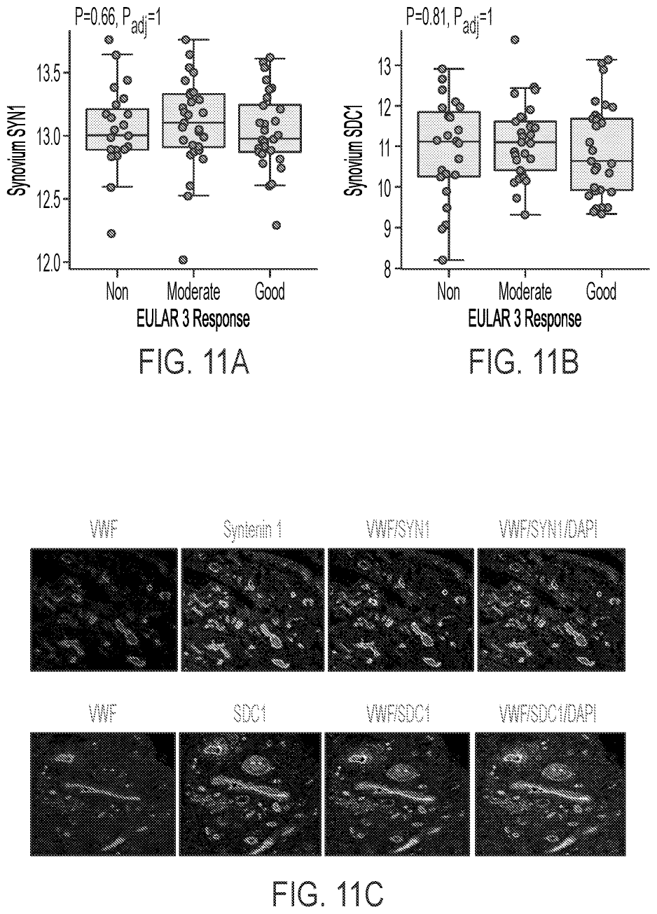

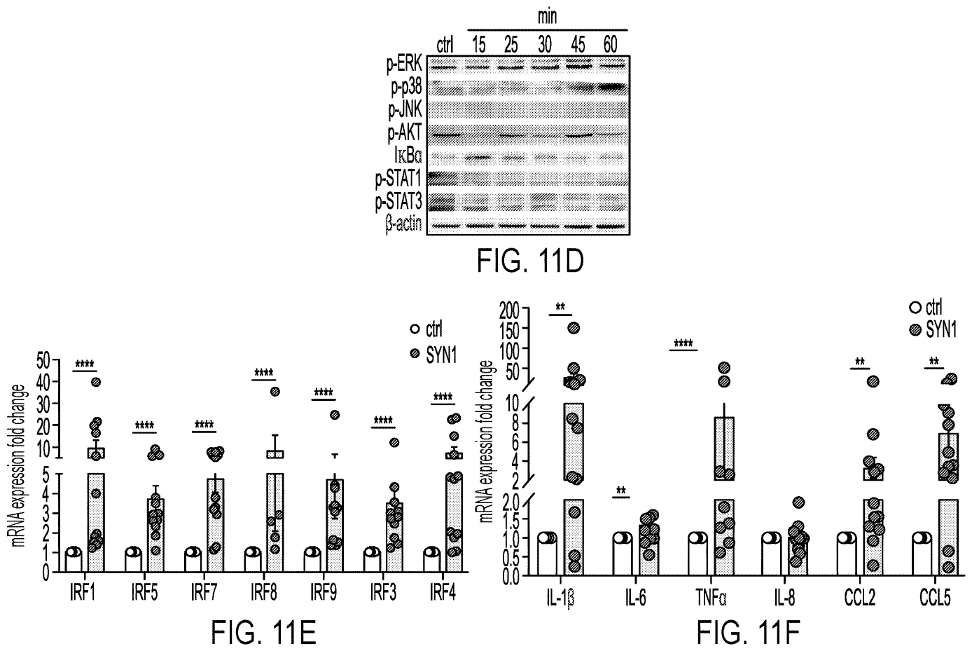

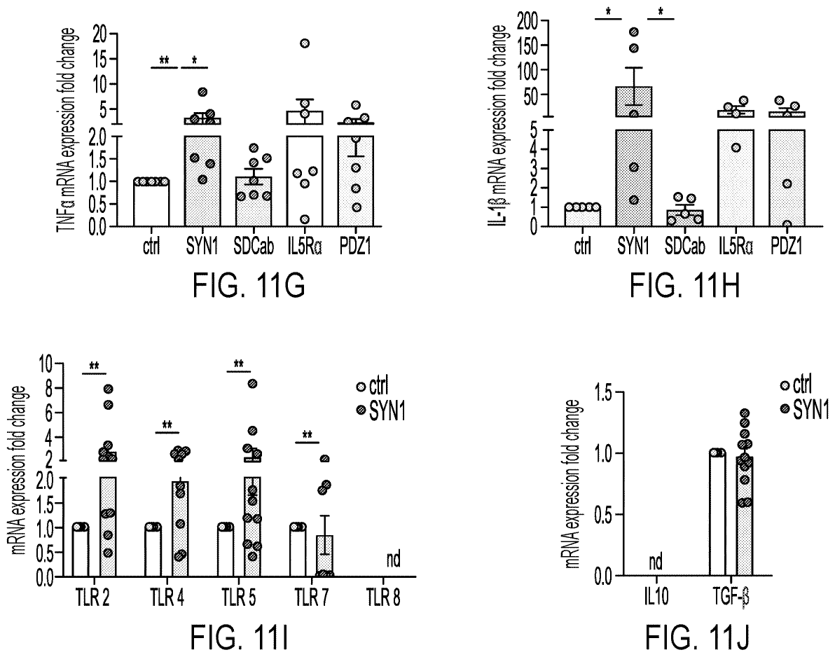

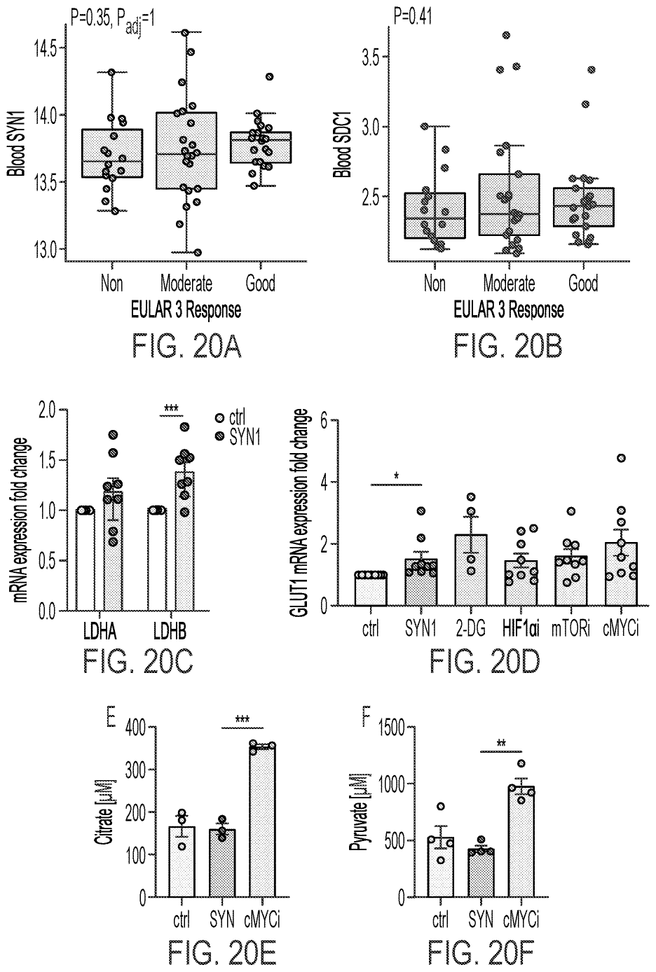

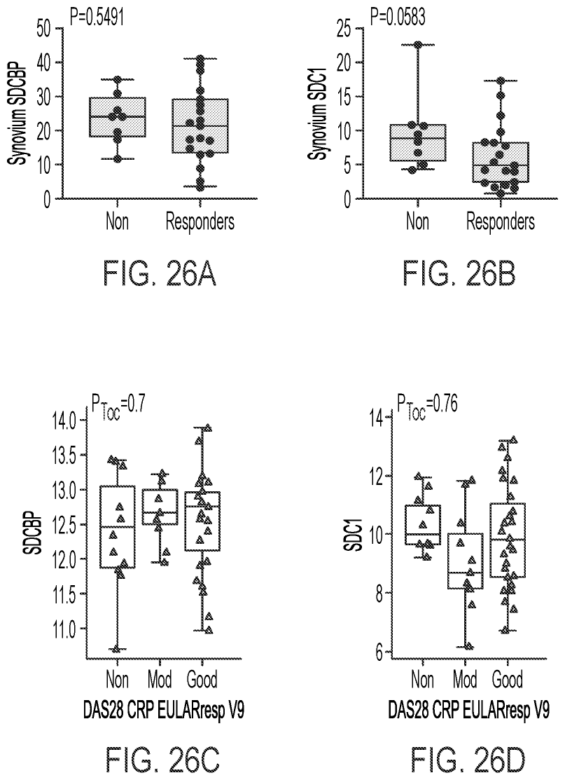

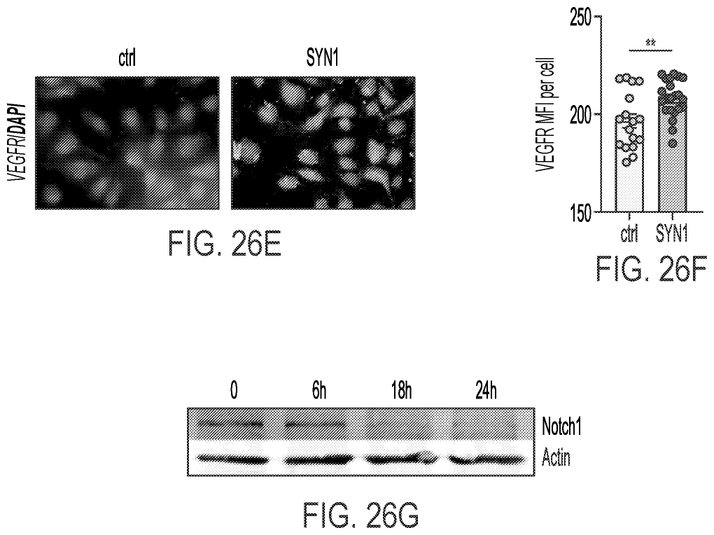

Results. Syntenin-1 and SDC-1 are co-expressed on RA ST endothelial cells and Syntenin-1 amplifies inflammatory reconfiguration in endothelial cells. RNA-seq data revealed that the expression of Syntenin-1 and SDC-1 in RA ST and blood were comparable in RA patients that were nonresponsive compared to those with moderate (DAS28 change≤1.2 and >0.6) and good response (DAS28 change>1.2) (FIGS. 11A-B and FIGS. 20A-B). Corroborating these findings, RA synovial Syntenin-1 and SDC-1 transcriptomes were unchanged in RA patients that displayed good response to anti-TNF (Certolizumab, FIG. 26A-B), and anti-IL-6R Ab (Tocilizumab. FIG. 26C-D) relative to non-responders. Intriguingly, Syntenin-1 and SDC-1 were co-localized on RA ST endothelial cells, demonstrating that cells producing Syntenin-1 were also responsive to its stimulation (FIG. 11C). Human umbilical vein endothelial cells (HUVECs) activated by Syntenin-1 exhibited ERK and p-38 MAPK signaling together with transient IκB degradation, while JNK, AKT, STAT1/3 cascades were unaffected (FIG. 11D).

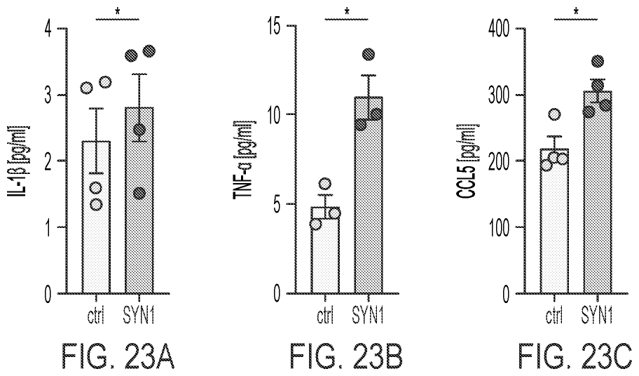

Moreover, expression of a wide range of transcription factors (IRF1/3/4/5/7/8/9) along with inflammatory mediators including IL-1p, IL-6, TNF, IL-8, CCL2, and CCL5 were upregulated at the transcriptional and translational levels in HUVECs reprogrammed by Syntenin-1 (FIGS. 11E-F, FIGS. 23A-C). Notably, while SDC-1 Ab constrained Syntenin-1-induced IL-1β and TNF transcription, blockade of IL-5R or PDZ1 did not influence this process (FIGS. 11G-H). Syntenin-1 activation also augmented HUVECs responsiveness to TLR ligands by advancing both the cell surface (TLR2/4/5) and the endosomal TLRs (TLR7) (FIG. 11I). In contrast, the pro-repair phenotype, IL-10, and TGFβ, were uninvolved in HUVECs remodeled by Syntenin-1 (FIG. 11J). In short, endothelial cells exposed to Syntenin-1 display a strong inflammatory profile that is primarily dependent on SDC-1 ligation.

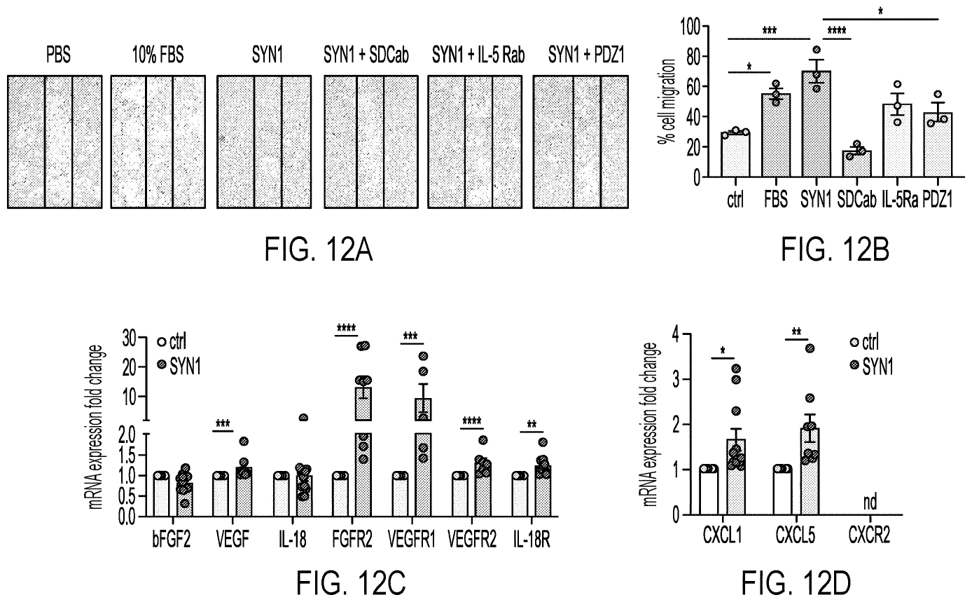

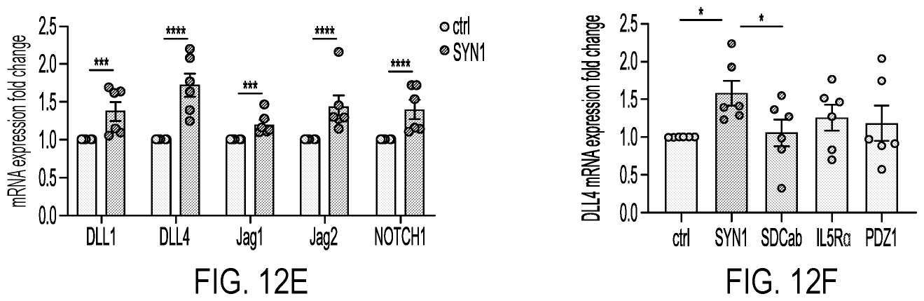

Syntenin-1 is responsible for endothelial cell migration and expression of pro-angiogenic factors. Given that Syntenin-1 and SDC-1 are colocalized on RA synovial vasculature, other manifestations of this pathway were examined on HUVECs as RA endothelial cell substitutes. The results show that endothelial cells migrate in response to Syntenin-1 via SDC-1 or PDZ1, which was unaffected by anti-IL-5R antibody (Ab) (FIGS. 12A-B). Further, transcription of numerous proangiogenic factors was markedly expanded in HUVECs reconfigured by Syntenin-1 which included VEGF, CXCL1, CXCL5, DLL1, DLL4, JAG1, and JAG2 (FIGS. 12C-E). Consistently, levels of FGFR2, VEGFR1/2, IL-18R, and Notch1 were also amplified in HUVECs through Syntenin-1 exposure (FIGS. 12C-E).

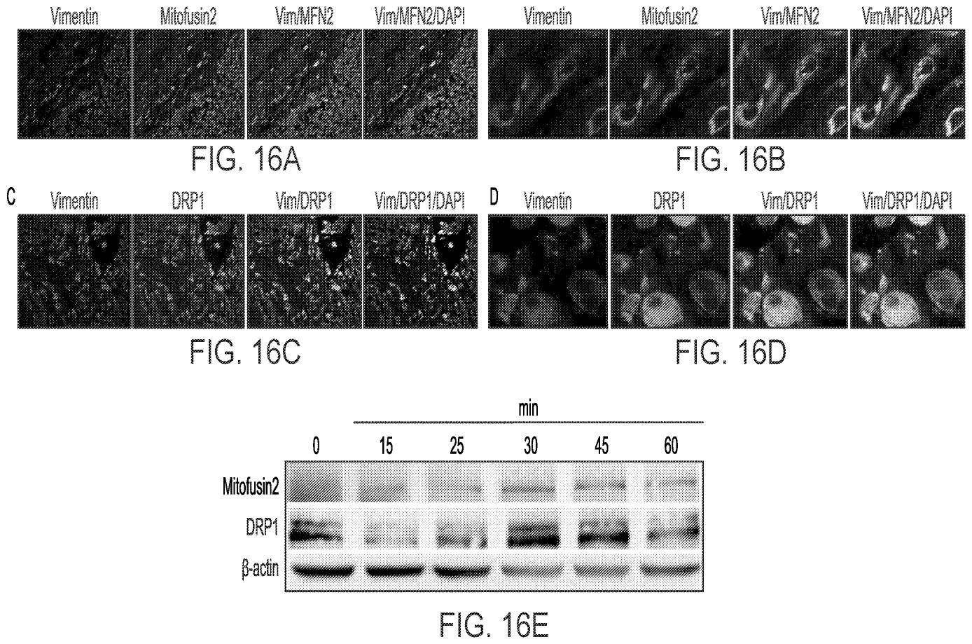

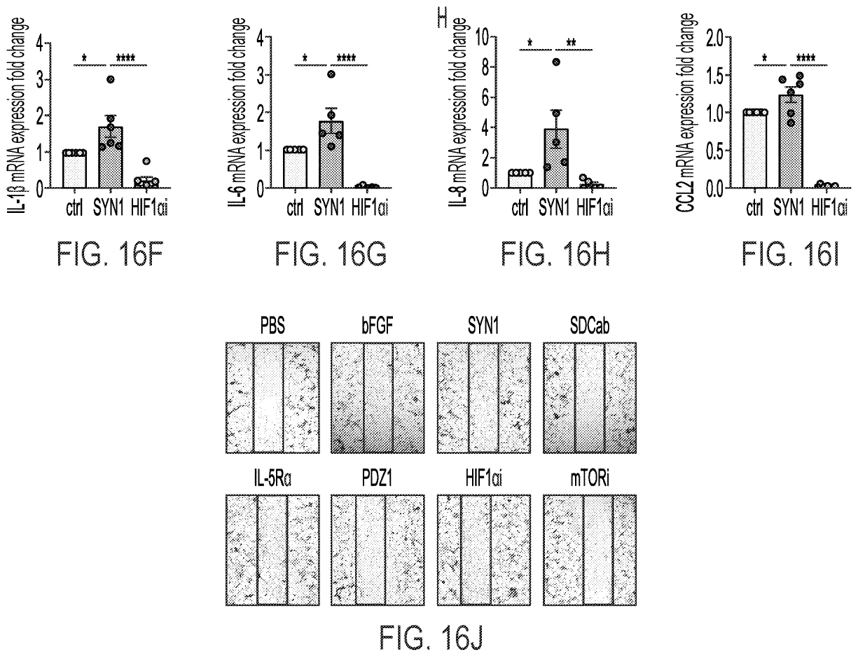

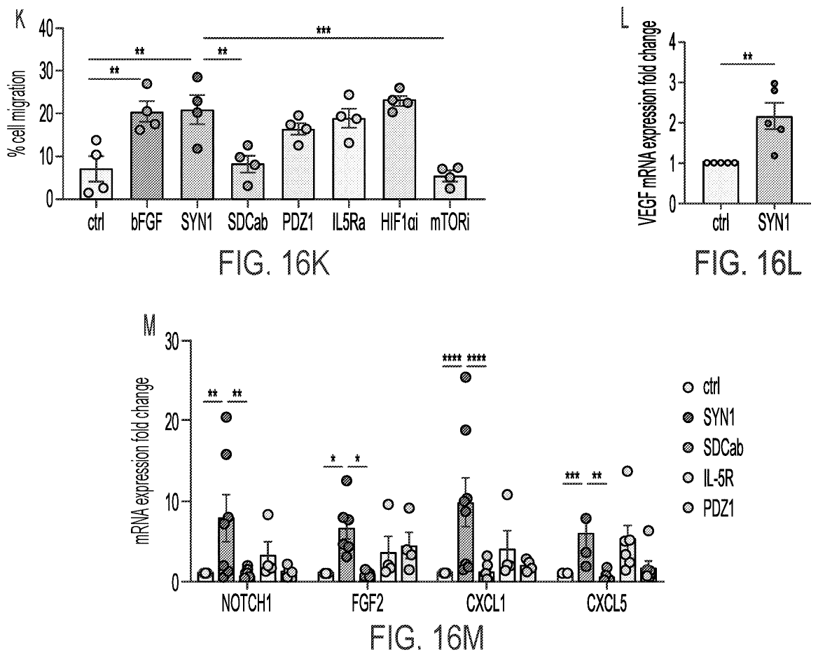

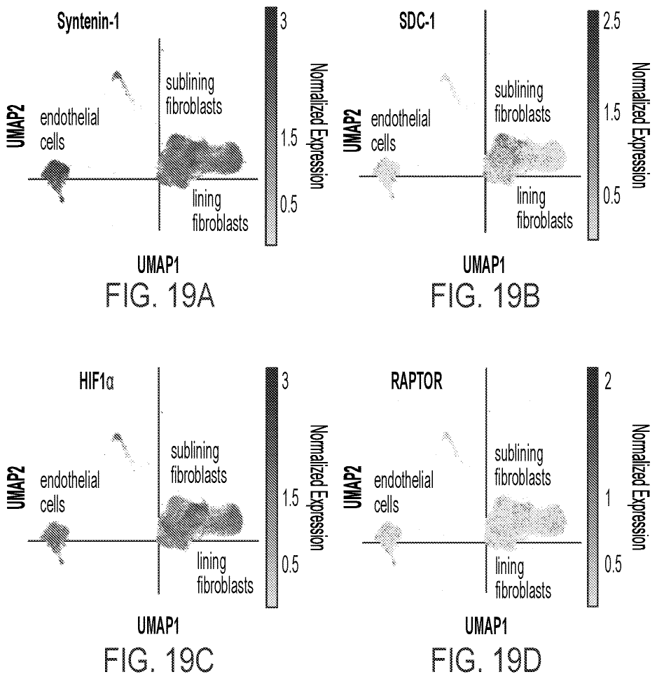

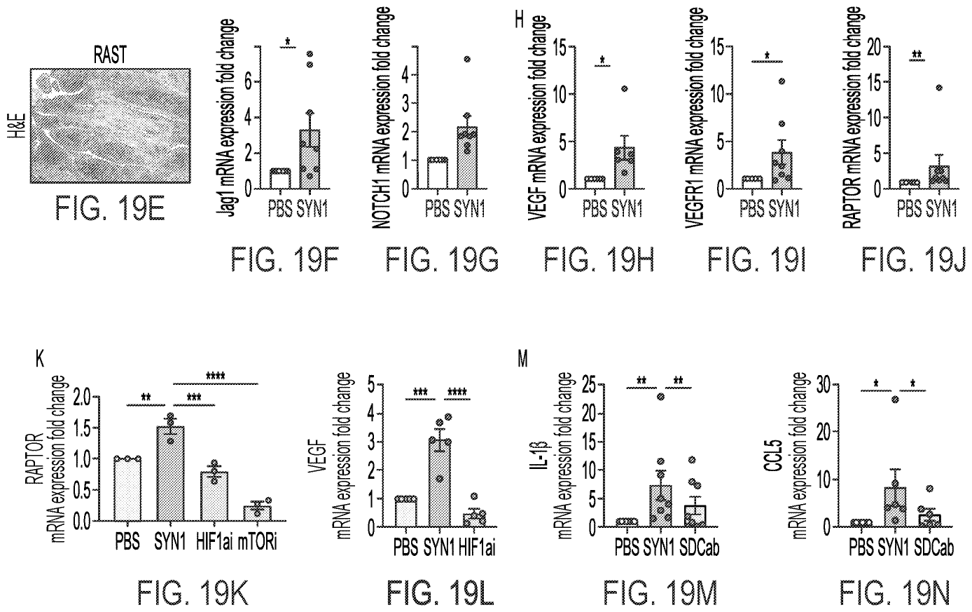

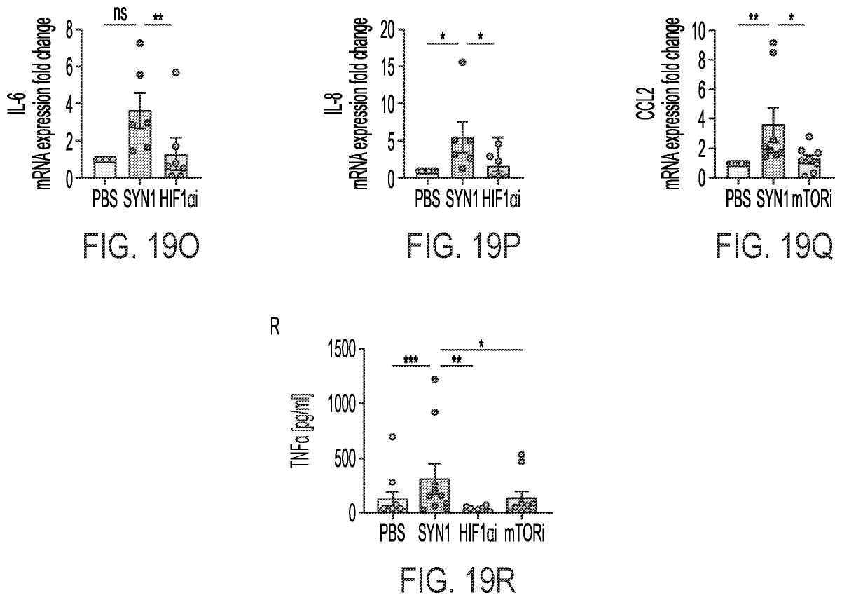

Data generated in RA ST explants and/or FLS highlighted the significance of VEGF & VEGFR1/2 and JAG1/Notch1 in Syntenin-1-induced pathology (FIG. 19G, 19I, and FIG. 16L-M). In agreement, VEGFR1/2 and Notch1, as well as their complementary ligands, were highly expressed in endothelial cells in response to Syntenin-1 (FIG. 12C, 12E, FIG. 26E-G). However, in some instances, either the ligand (bFGF2, IL-18) or the receptor (CXCR2) remained undetected (FIG. 12C-D). Similar to endothelial cell-enhanced inflammation and infiltration. SDC-1 was responsible for Synteinin-1-mediated DLL4 transcription (FIG. 12F). Taken together, angiogenesis is advanced both directly and indirectly by ligation of Syntenin-1 to SDC-1⁺ endothelial cells in part through VEGFR and Notch1 networks.

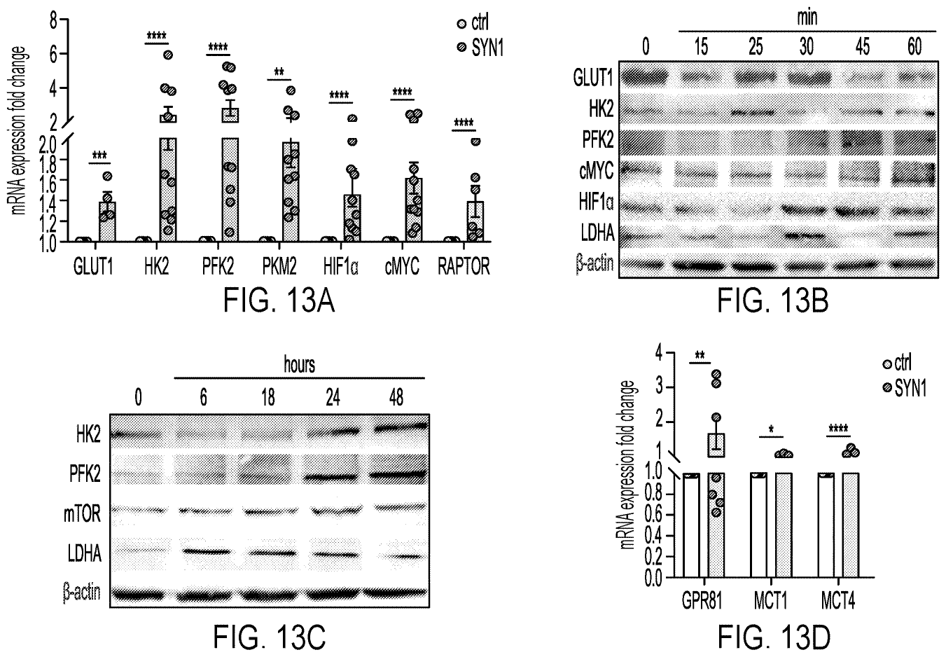

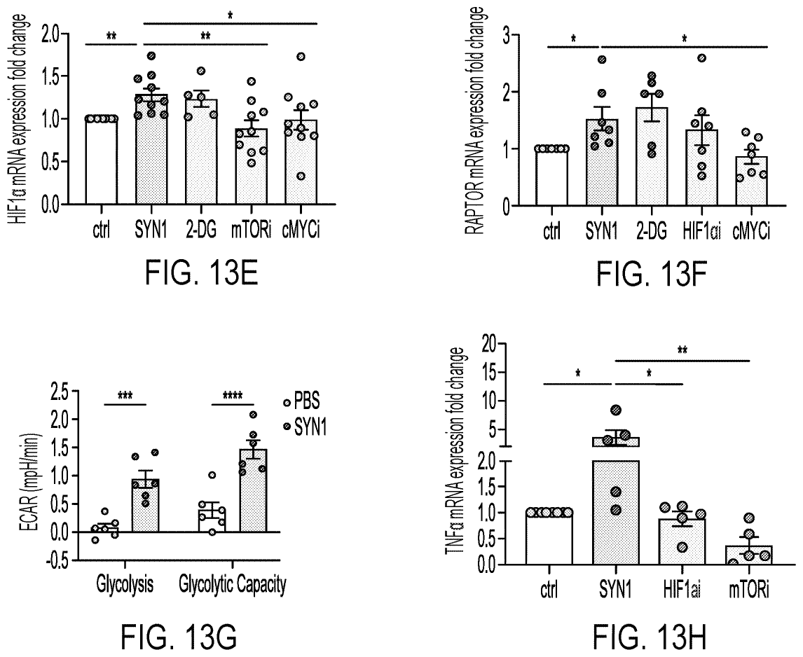

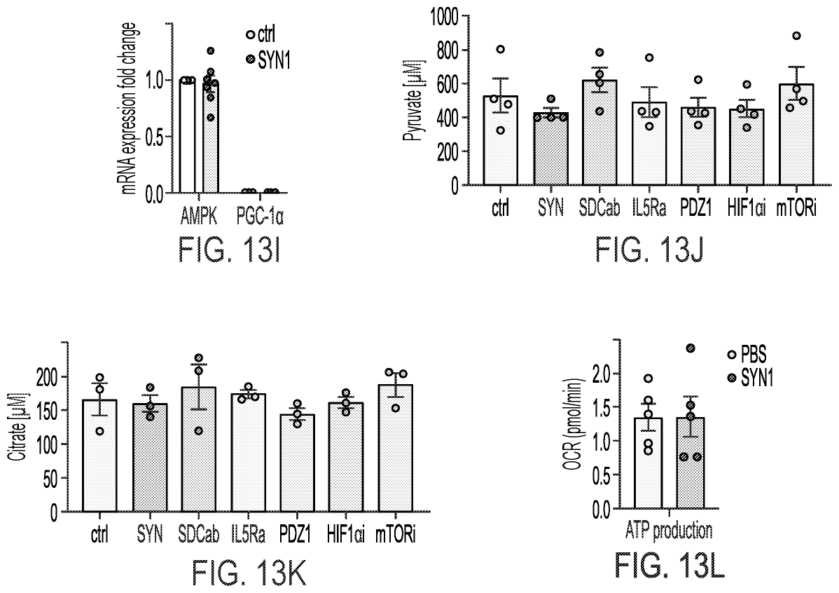

HIF1α and RAPTOR activation promotes Syntenin-1 metabolic reprogramming in endothelial cells. Next, experiments were conducted to determine whether the endothelial inflammatory landscape is influenced by metabolic rewiring by Syntenin-1. Syntenin-1 reprogramming of endothelial cells resulted in transcriptional upregulation of a wide range of glycolytic intermediates, GLUT1, HK2, PFK2, PKM2, HIF1α, cMYC, and RAPTOR (FIG. 13A). Contrary to transcriptional upregulation of GLUT1, its translation levels were unaffected by Syntenin-1 in HUVECs (FIG. 13B). While HK2, PFK2, and LDHA protein levels were transiently enhanced at short-term Syntenin-1 activation, and their levels were more stably elevated in HUVECs following 24 h and 48 h of stimulation (FIGS. 13B-C). Moreover, HIF1α, cMYC, and mTOR/RAPTOR protein levels were enriched in Syntenin-1 reprogrammed HUVECs (FIGS. 13B-C).

Interestingly, lactate specific receptor on endothelial cells, GPR81, as well as its transporters MCT1 (importer) and MCT4 (exporter) were potentiated by Syntenin-1 (FIG. 13D). The data show that following Syntenin-1 stimulation, there is dynamic glycolysis that occurs by lactate being sensed through heightened endothelial GPR81 frequency as well as the activity of the transporters directing its import or export.

Additionally, Syntenin-1-elevated HIF1α and RAPTOR expression levels are suppressed by cMYCi, whereas HIF1α can also be dysregulated by mTORi (FIGS. 13E-F). In endothelial cells reprogrammed by Syntenin-1, HIF1α, and RAPTOR signaling are linked to the amplification of glycolysis and glycolytic capacity as well as the inflammatory phenotype (FIGS. 13G-H). Corroborating with this notion, TNF expression was diminished by HIF1αi and mTORi in Syntenin-1-reconfigured endothelial cells, yet cMYCi did not replicate this function (FIG. 13H).

The oxidative metabolites, AMPK, PGC-1a, and citrate were unaltered in endothelial cells reprogrammed by Syntenin-1 and consequentially unchanged by HIF1αi and mTORi therapy (FIGS. 13I-K), cMYCi treatment was capable of advancing pyruvate and citrate levels in Syntenin-1 reprogrammed endothelial cells (FIGS. 20E-F). Distinct from the robust induction of glycolysis and its intermediates delineated in endothelial cells reconfigured by Syntenin-1, OCR, and oxidative metabolites were uninvolved in these cells (FIGS. 13I-L). Altogether endothelial cells are metabolically reprogrammed by Syntenin-1 in part through HIF1α and mTOR activation.

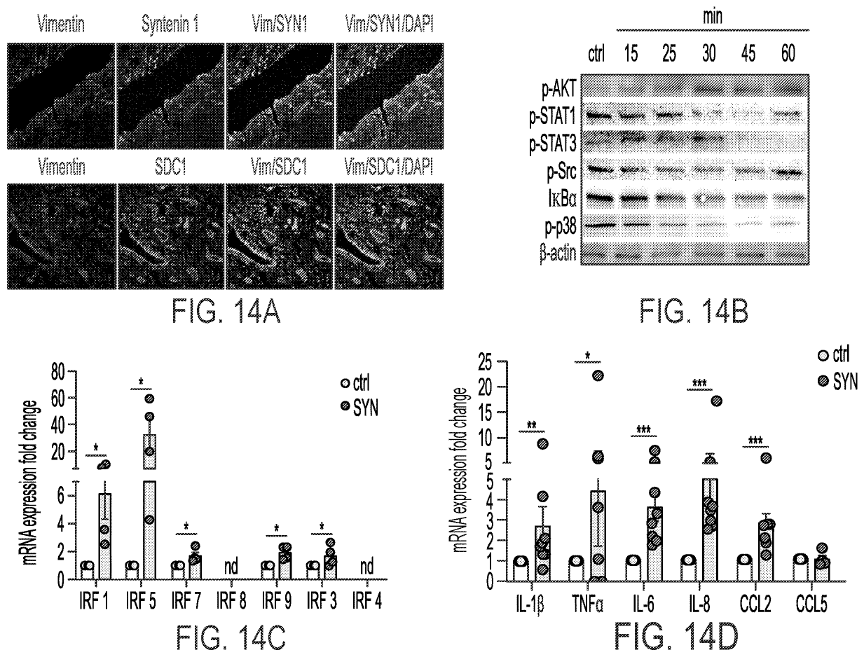

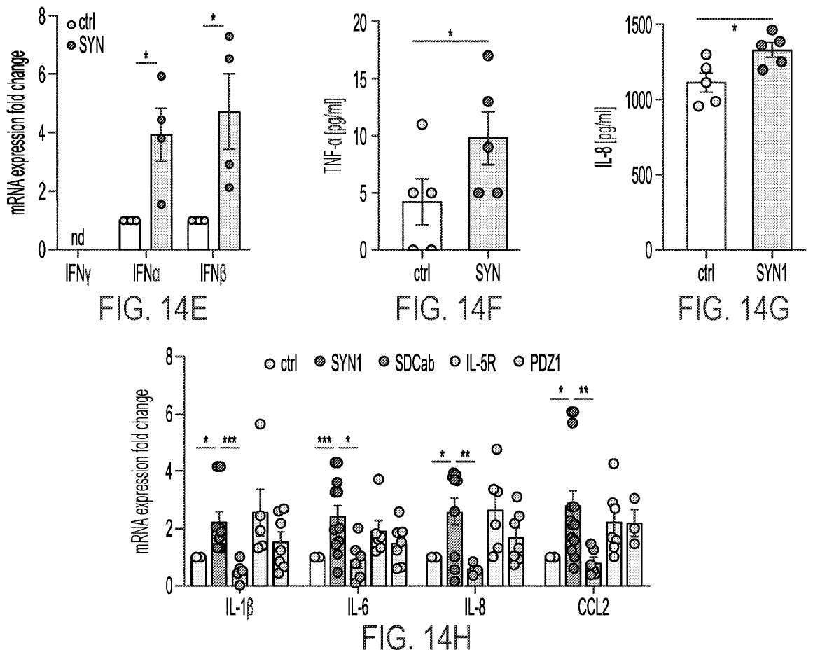

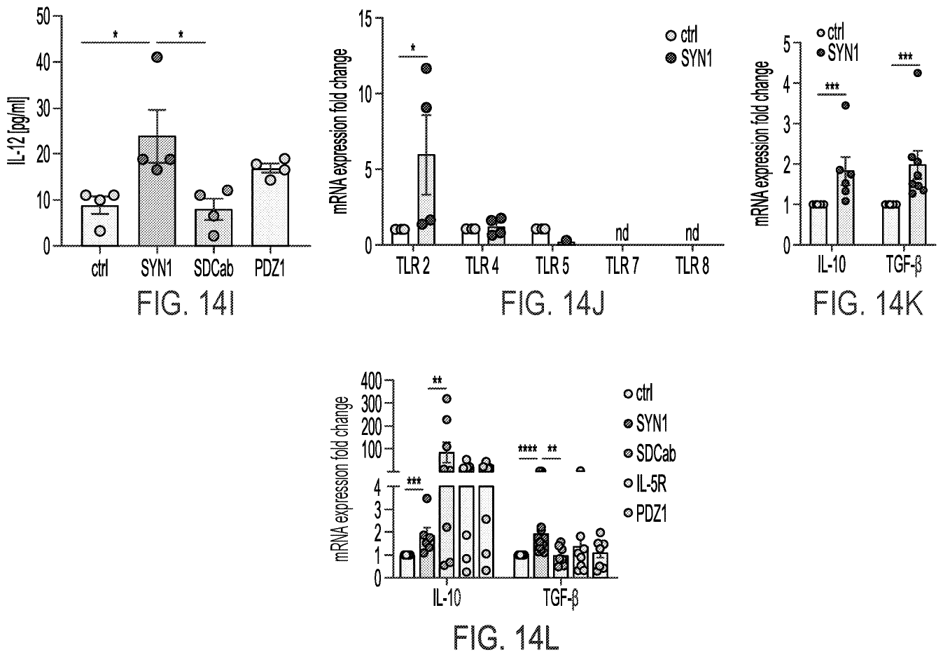

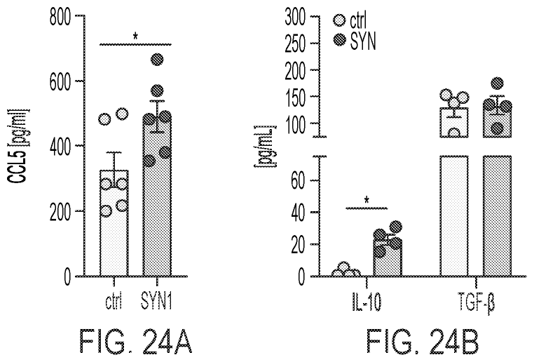

RA FLS remodeled by Syntenin-1 display inflammatory imprint. Morphological studies elucidated that both Syntenin-1 and SDC-1 are co-localized on Vimentin⁺RA FLS (FIG. 14A). Syntenin-1 stimulated RA FLS signal through AKT and NF-κB with no effect on STAT1/3, Src, or p38 activation (FIG. 14B). Reprogramming of RA FLS by Syntenin-1 coincides with expanded IRF1/5/7/9/3 along with a robust inflammatory phenotype that reveals IL-1p, IL-6, TNF, IL-8, CCL2, CCL5, IFNα, and IFNβ induction of transcriptome and/or protein levels (FIGS. 14C-G, FIG. 24A). The inflammatory remodeling of RA FLS by Syntenin-1 and enhancement of IL-1p, IL-6. IL-8. CCL2, and IL-12 was impaired by SDC-1 Ab but not PDZli (FIG. 14H-I).

However, unlike endothelial cells, TLRs amplification in RA FLS exposed to Syntenin-1 was restricted to TLR2 (FIG. 14J). Also distinct from HUVECs, RA FLS remodeled by Syntenin-1 show higher IL-1β expression, unlike TGFβ which was unaffected in both cell types (FIG. 14K-L; FIG. 24B). Collectively, the results show that RA FLS reprogramming by Syntenin-1 is accompanied by a predominant inflammatory phenotype that exceeds the pro-repair profile.

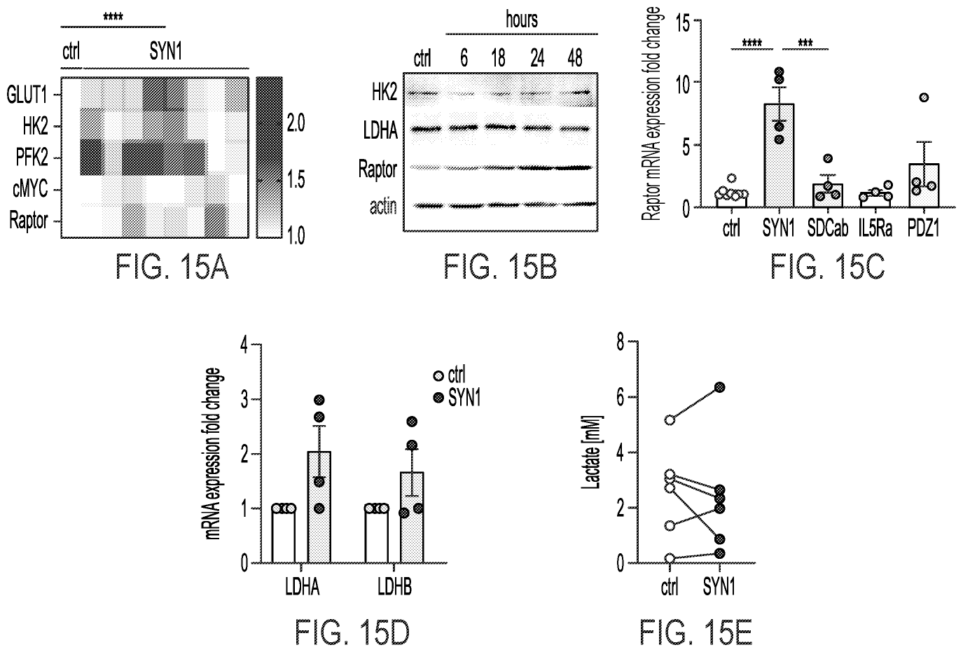

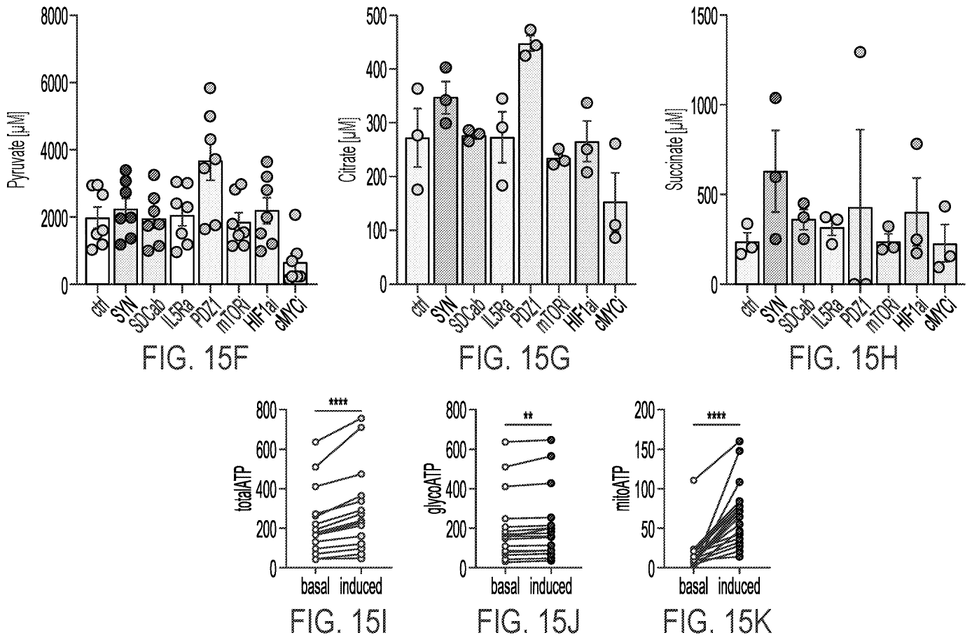

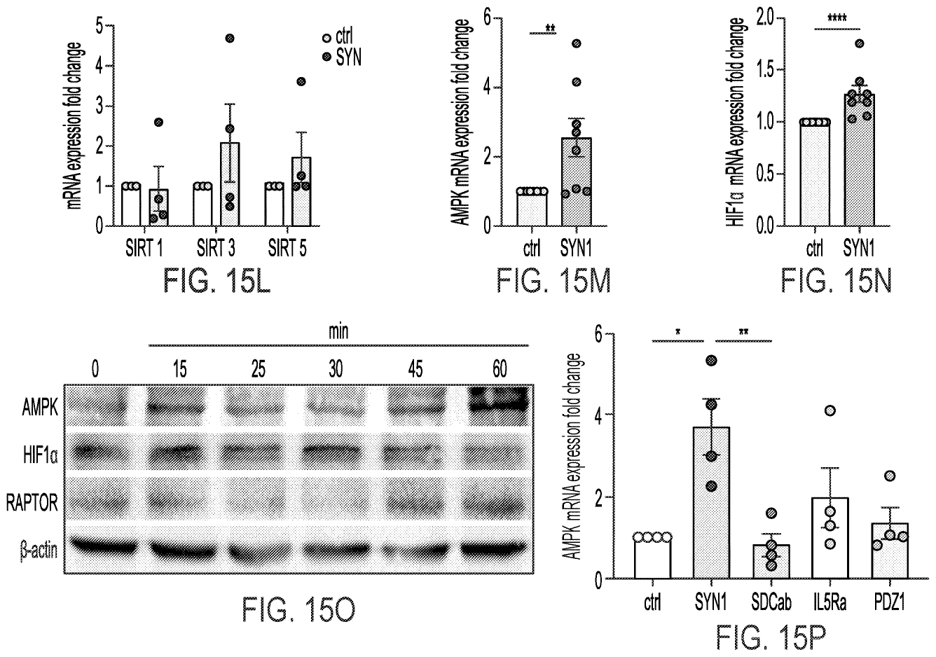

RA FLS remodeled by Syntenin-1 have an uncommon metabolic profile. Next, the metabolic functionality of RA FLS remodeling by Syntenin-1 was analyzed to characterize its participation in different implications. In RA FLS, Syntenin-1 stimulation was capable of promoting a modest transcriptional induction of GLUT1, HK2, PFK2, cMYC, and RAPTOR, but not PKM2 (FIG. 15A, FIG. 2IA-F). Furthermore, elevated RAPTOR protein expression was captured in RA FLS reprogrammed by Syntenin-1 following short (45-60 min) and long exposure (18 h-48 h) (FIGS. 15B, 15O). RAPTOR expression levels were counteracted by SDC-1 Ab but not IL-5R Ab or PDZ1i in RA FLS rewired by Syntenin-1 (FIG. 15C). In parallel, neither lactate catalyzing enzymes (LDHA or LDHB) nor accumulation of pyruvate or lactate were impacted in Syntenin-1-remodeled RA FLS (FIGS. 15D-F).

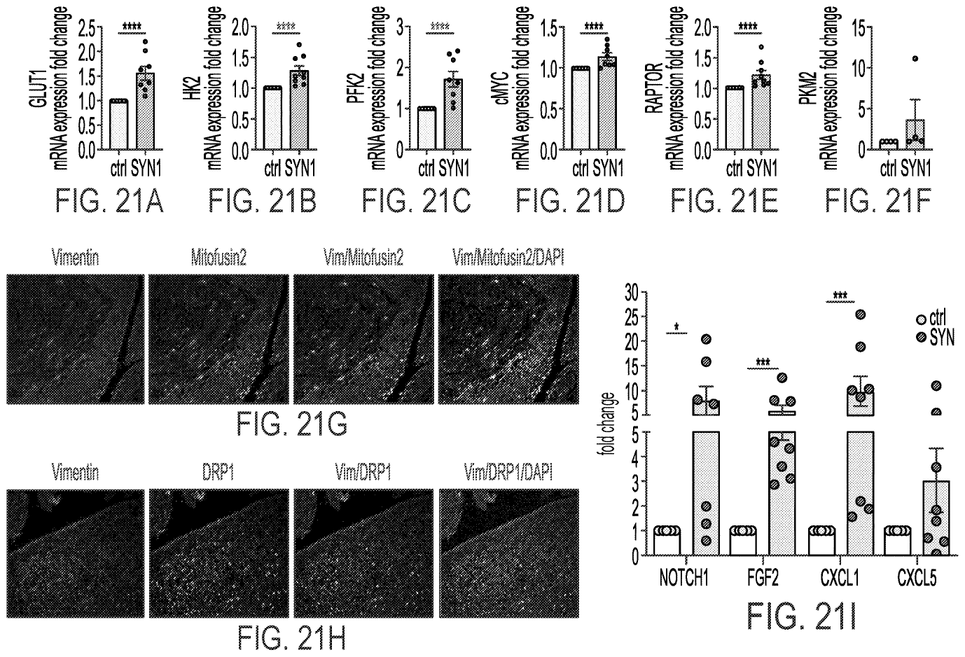

Concurrently, secretion of oxidative metabolites including citrate and succinate was unaffected in Syntenin-1-rewired RA FLS (FIGS. 15G-H). Nonetheless, RA FLS exposed to Syntenin-1 showed a marked increase in total ATP levels which was accompanied by a modest glycoATP and a more intense mitoATP activity (FIG. 15I-K). In evaluating other oxidative intermediates in Syntenin-1-reprogrammed RA FLS, while SIRT1/3/5 were unaffected (FIG. 15L), transcription and translation levels of AMPK and HIF1α were significantly potentiated (FIGS. 15M-O) and further manipulated by SDC-1 ligation (FIG. 15P). Despite the lack of succinate accumulation in Syntenin-1-remodeled RA FLS (FIG. 15H), mitochondrial oxidative phosphorylation is shown to be related to AMPK and HIF1α activation (Salminen A, et al. Biogerontology. 2016; 17:655-680: Li H. et al. Am J Physiol Renal Physiol. 2015; 309:F414-428; and Hwang A B, et al. Proc Natl Acad Sci USA. 2014; 111:E4458-4467). The results also show that the stark mitoATP activity observed in Syntenin-1-reprogrammed RA FLS (FIG. 15K) was coupled with mitochondrial fusion and fission signified by escalated Mitofusin-2 and DRP1 (FIGS. 16A-E). The results also showed that Mitofusin-2 and DRP1 are colocalized on Vimentin⁺RA FLS and Syntenin-1 exposure amplifies their protein expression (FIGS. 16A-E, FIGS. 21G-H). Remarkably, HIF1αi strongly impaired the inflammatory reconfiguration of RA FLS by Syntenin-1 and led to the downregulation of IL-10, IL-6, IL-8, and CCL2 transcription (FIGS. 16F-I). Meanwhile, Syntenin-1-driven RA FLS migration was intercepted by SDC-1 Ab and mTORi but not HIF1αi (FIGS. 16J-K). Extending the findings with HUVECs, RA FLS exposed to Syntenin-1 revealed transcriptional enrichment of pro-angiogenic mediators including VEGF and/or Notch1, FGF2. CXCL1, and CXCL5 which were singularly constrained by SDC-1 Ab (FIGS. 16L-M, FIG. 2I1). Taken together, while metabolic dysregulation via HIF1α manipulates Syntenin1 expanded inflammatory network in RA FLS, mTOR signaling is involved in RA FLS migration in response to Syntenin-1.

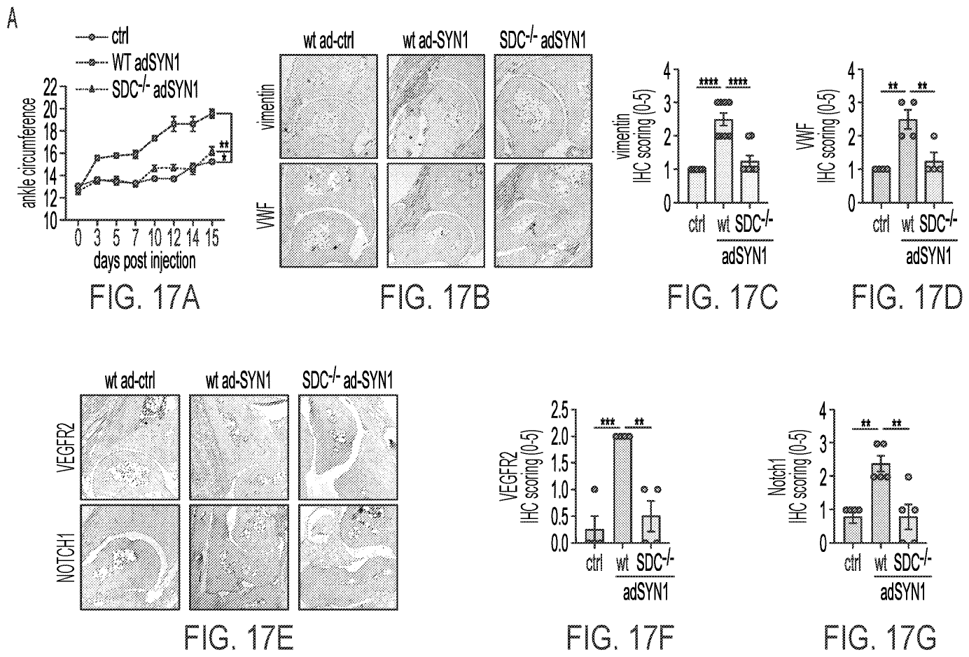

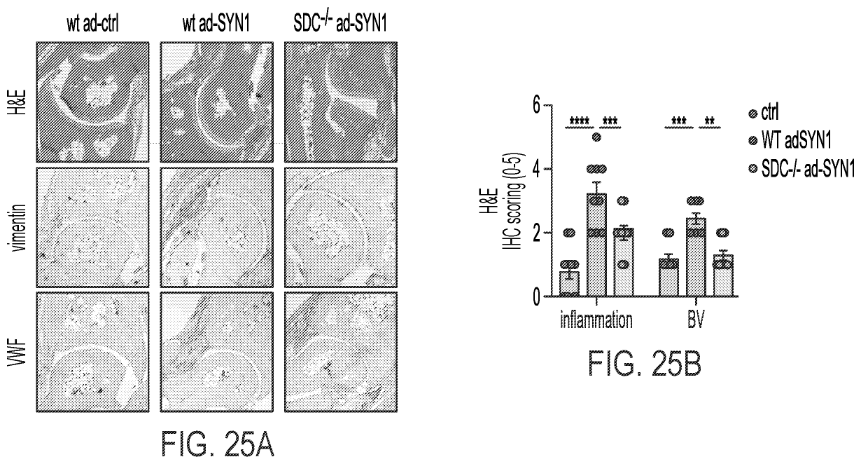

Syntenin1-induced pathology is mitigated by SDC-1 disruption and RAPTOR or HIF1α deactivation. Local injection of adenovirus (ad)-Syntenin-1 resulted in progressive arthritic joint inflammation in wild-type mice compared to SDC-1 KO mice that received ad-Syntenin-1 or ad-Ctrl administration (FIG. 17A). Arthritic joint swelling manifested by ectopic expression of Syntenin-1 in WT mice was manifested by escalated joint inflammation and blood vessel formation (BV) was accompanied by the expansion of Vimentin*fibroblasts and VWF⁺ endothelial cells which were obstructed in SDC-1^{-/-} animals (FIGS. 17B-D, FIGS. 25A-B). Morphological and transcriptome studies recapitulate the importance of VEGFR1/2, Notch1, RAPTOR, and HIF1α pathways in Syntenin1-induced arthritis and their dysregulation in SDC-1 deficient mice compared to the control animals (FIGS. 17E-I). In line with these findings, expression of GLUT1 and HK2 was upregulated in joint Vimentin+fibroblasts and VWF⁺ endothelial cells in the wild-type Ad-Syntenin-1 arthritic mice compared to ctrl or SDC1−/− ad Syntenin-1 groups. For example, GLUT1 was found co-expressed on Vimentin⁺fibroblasts and VWF⁺ endothelial cells in Syntenin-1 arthritic joints. WT and SDC^{-/-} mice were injected intra articularly with ad-ctrl (ctrl) or adSYN1 (3×10¹⁰ viral particles/ankle) on days 0, 7, and 14 and joint circumference was monitored over 15 days (n=10 mice/group). Ankles from non-arthritic WT ctrl and WT or SDC^{-/-} mice injected with ad-SYN1 were co-stained with Vimentin or VWF in combination with GLUT1 by immunofluorescence staining, n=3. Also, HK2 was found co-expressed on Vimentin+fibroblasts and VWF⁺ endothelial cells in Syntenin-1 arthritic joints. WT and SDC^{-/-} mice were injected intra articularly with ad-ctrl (ctrl) or adSYN1 (3×10¹⁰ viral particles/ankle) on days 0, 7, and 14 and joint circumference was monitored over 15 days (n=10 mice/group). Ankles from non-arthritic WT ctrl and WT or SDC^{-/-} mice injected with adSYN1 were co-stained with Vimentin or VWF in combination with HK2 by immunofluorescence staining, n=3.

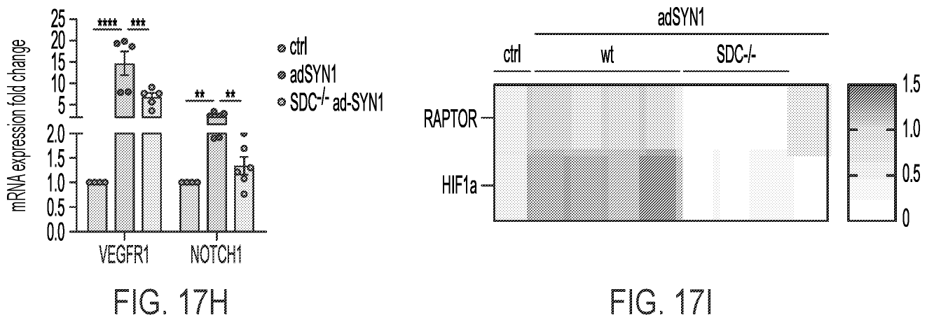

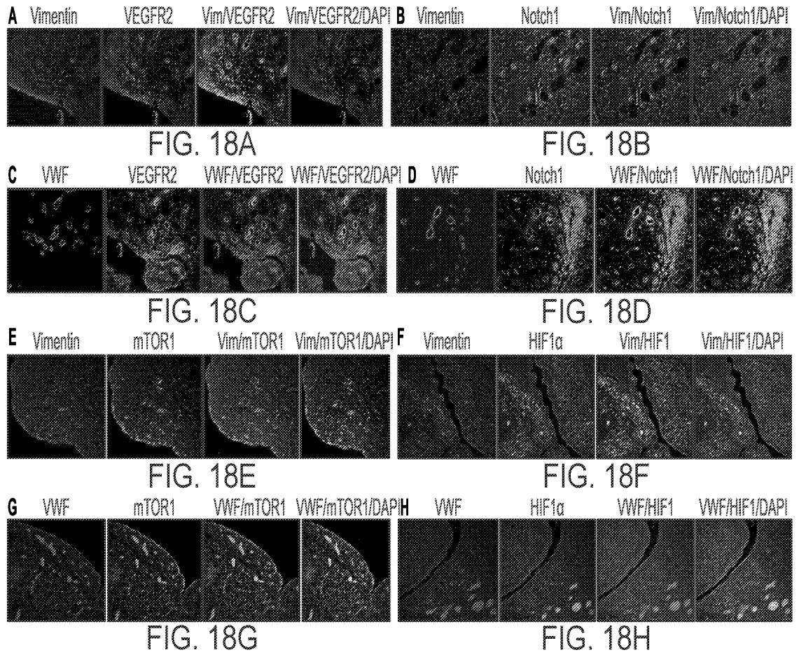

Corroborating these findings in Syntenin1-induced arthritis, VEGFR2 (FIGS. 18A, 18C), Notch1 (FIGS. 18B, 18D), RAPTOR (FIGS. 18E, 18G), and HIF1α (FIGS. 18F, 18H) are shown to be co-expressed on Vimentin⁺RA FLS and VWF⁺RA endothelial cells. At the onset of these studies, the interplay between endothelial cells and RA FLS was assessed in response to Syntenin-1 in coculture. However, because HUVECs require a high growth factor milieu for optimal proliferation, the coculture is taken over by RA FLS leading to endothelial cell death. Hence these experiments were performed in explants where RA FLS are in direct contact with the endothelium (FIG. 19A).

Intriguingly, in Syntenin-1-induced arthritis, the results show that VEGFR2 (FIG. 18A, 18C), Notch1 (FIG. 18B, 18D), mTOR (FIG. 18E, 18G), and HIF1α (FIG. 18F, 18H) are co-expressed on Vimentin⁺RA FLS and VWF⁺RA endothelial cells by IF staining. Authenticating the morphological findings, single-cell RNAseq displays that Syntenin-1 and HIF1α are widely expressed on RA FLS and ST endothelial cells, while SDC-1 and RAPTOR are modestly presented on these cell types (FIG. 19A-D). At the onset of these studies, the interplay between endothelial cells and RA FLS in response to Syntenin-1 in coculture was assessed. However, because HUVECs require a high growth factor milieu for optimal proliferation, the coculture is taken over by RA FLS leading to endothelial cell death. Hence, these experiments were performed in explants where RA FLS are in direct contact with the endothelium (FIG. 19E).

To evaluate the functional significance and cross-regulation of VEGFR, Notch1, and inflammatory phenotype in connection with SDC-1 ligation or RAPTOR and HIF1α activation, RA explants were exposed to Syntenin-1 in the presence or absence of SDC-1 Ab, mTORi or HIF1αi. JAG1, Notch1. VEGF. VEGFR1, and RAPTOR transcription levels were amplified in RA ST explants stimulated by Syntenin-1 (FIGS. 19F-J). It was also observed that Syntenin-1-enriched RAPTOR expression was suppressed both by mTOR and HIF1α inhibitors (FIG. 19K). In light of these findings, HIF1αi was capable of negating the expression of VEGF (FIG. 19L) and numerous inflammatory mediators such as IL-6. IL-8, and TNF in RA explants in response to Syntenin-1 (FIGS. 19O-R). While SDC-1 Ab was responsible for restricting IL-1β and CCL5 transcription (FIGS. 19M-N), mTORi intercepted CCL2 transcription and TNF secretion in RA explants exposed to Syntenin-1 (FIGS. 19Q-R). Collectively, the data show that Syntenin-1-escalated inflammatory and/or proangiogenic landscapes in endothelial cells, RA FLS, and RA explants are primarily modulated by SDC-1 and HIF1α. Whereas mTOR activity has a more restricted influence on Syntenin-1-expanded inflammatory profile in endothelial cells and RA explants as well as migration of RA FLS.

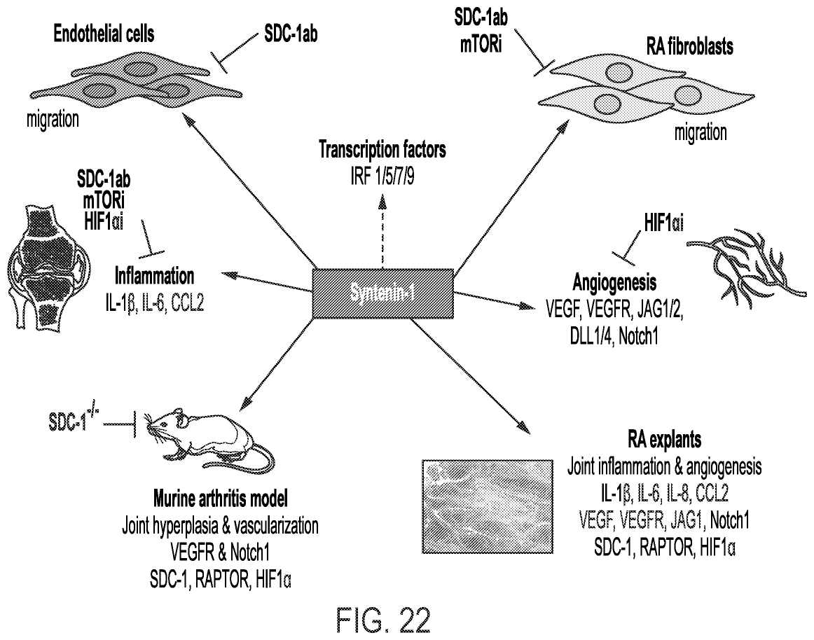

The results demonstrate the pathology of the RA synovial fluid protein, Syntenin-1, that can reprogram endothelial cells and RA FLS by molding their inflammatory and angiogenic landscapes with metabolic activity. The findings show that Syntenin-1 remodels the inflammatory imprint of endothelial cells and RA FLS by activating IRF1/5/7/9 alongside expanding the transcription of IL-1β, IL-6, and CCL2 via SDC-1 ligation, HIF1α, and/or mTOR activation. Nevertheless, the Syntenin-1-driven metabolic reconfiguration is quite distinct in endothelial cells relative to RA FLS. Syntenin-1 rewired endothelial cells display elevated glycolytic capacity with robust activation of RAPTOR and HIF1α, while the mitochondrial oxidative phosphorylation is unaffected as corroborated by unchanged OCR and AMPK levels. Also, RA FLS reprogrammed by Syntenin-1 showed a modest glycoATP together with a more prominent mitoATP activity. This RA FLS phenotype is signified by elevated oxidative stress and altered mitochondrial dynamics facilitated through amplified 15 AMPK, HIF1α, and Mitofusin-2, or DRP1. These findings in endothelial cells and RA FLS are recapitulated in murine arthritic joints, and RA explants, where Syntenin-1 plays an important role in guiding the inflammatory and angiogenic networks through VEGFR and Notch1 via HIF1α and RAPTOR involvement. In short, Syntenin-1 can induce RA pannus through its ability to link the inflammatory, angiogenic, and metabolic networks of endothelial cells with RA FLS (FIG. 22).

Syntenin-1 and SDC-1 were discovered by RNA-seq studies, where their expression in RA STs was linked to CD68⁺sublining cells, ESR, and/or ultrasound ST thickness (Meyer A, et al. Ann Rheum Dis. 2023; and Lewis M J, et al. Cell reports. 2019; 28:2455-2470 e2455). The Syntenin-1/SDC-1 pathway became a more attractive therapeutic target when its expression was found to be unaffected in responders who were treated with DMARDs or biologics. These findings led to testing whether Syntenin-1 and SDC-1 expression expanded beyond RA myeloid cells. Intriguingly, Syntenin-1 and SDC-1 are colocalized on RA ST VWF⁺ endothelial cells and Vimentin⁺RA FLS as revealed by single-cell RNAseq analysis.

The results also show that endothelial cells reprogrammed by Syntenin-1 displayed a robust inflammatory phenotype that was exhibited by activation of ERK, p38, NF-κB as well as transcriptional upregulation of IRFs, IL-1, IL-6, IL-8, TNF, CCL2, CCL5 and numerous TLRs. In contrast, the pro-repair phenotype, through IL-10 and TGFβ, was uninvolved in endothelial cells exposed to Syntenin-1. Syntenin-1 ligation to SDC-1, directly advanced endothelial cell migration. Concurrently, upregulation of the proangiogenic factors (VEGF, DLL1/4, JAG1/2) and their complementary receptors, VEGFR and Notch1, supported the indirect role of Syntenin-1 on angiogenesis. Earlier studies have reported that Syntenin-1 interaction with VEGFR and ephrin-B2 in endothelial cells expands VEGF-mediated angiogenesis (Tae N, et al. Oncotarget. 2017; 8:38886-38901). Others have shown that the production of Insulin Growth Factor Binding Protein-2 (IGFBP-2) from melanoma cells activated by Syntenin-1 is responsible for VEGF secreted from HUVECs (Das S K. et al. Cancer Res. 2013; 73:844-854). Previous reports also demonstrate that overexpression of SDC-1 in mesothelioma cells dysregulates endothelial cell migration and tube formation (Javadi J, et al. Cancers (Basel). 2021; 13). Distinctly, the findings disclosed herein demonstrate the importance of SDC-1 ligation for endothelial cell migration and angiogenic factor expression in response to Syntenin1.

Extensive similarities were noted between RA MΦs and endothelial cells reprogrammed by Syntenin-1, as both cell types demonstrated escalated ECAR activity that was accompanied by elevated GLUT1, HK2, PFK2, HIF1α, and RAPTOR (Meyer A, et al. Ann Rheum Dis. 2023). While OCR and AMPK levels were unaffected in endothelial reprogrammed by Syntenin-1, mitoATP, and AMPK transcription were reduced in Syntenin-1-differentiated Mis (Meyer A, et al. Ann Rheum Dis. 2023). In Syntenin-1 reconfigured endothelial cells the inflammatory imprint was reversed by HIF1αi and mTORi treatment. Whereas, the inflammatory and metabolic (CD14⁺CD86⁺GLUT1⁺) networks expanded in RA MΦs rewired by Syntenin-1 were exclusively impaired by mTORi primarily due to glucose uptake (Meyer A. et al. Ann Rheum Dis. 2023). It was found that endothelial cells reprogrammed by TNF were distinct from those rewired by Syntenin-1, as exposure to TNF resulted in a distinct profile that was exhibited by upregulated GLUT4, PFK2, and downregulated PDK4 along with enhanced ECAR and OCR (Xiao W, et al. Circ Res. 2021; 129:9-29). These authors delineated that the inflammatory and metabolic activity observed in TNF reconfigured endothelial cells was disrupted by blocking NF-κB and PFK2 function (Xiao W, et al. Circ Res. 2021; 129:9-29). Furthermore, mitochondrial pyruvate carrier inhibition enhanced PDK4 transcription and the inflammatory phenotype along with restraining OCR advanced by TNF-remodeled endothelial cells (Xiao W. et al. Circ Res. 2021: 129:9-29). Contrasting this observation, OCR was unchanged and disconnected from the inflammatory landscape detected in Syntenin-1 reprogrammed endothelial cells. Others have shown glycolysis activation via PFK2 is responsible for VEGF-induced angiogenesis (Wong B W, et al. EMBO J. 2017; 36:2187-2203), yet the findings disclosed herein show that HIF1α-induced signaling is responsible for VEGF expression and function in RA STs. RA FLS remodeled by Syntenin-1 and those differentiated by LPS/IFNγ were capable of activating AKT and NF-κB signaling as well as upregulating IRF1/5/7 along with IL-6, IL-8, and CCL2, which was reversed by SDC-1 Ab or 2-DG and IACS (Complexli), respectively (Umar S, et al. Cell Mol Life Sci. 2021). Syntenin-1 reprogramming did not impact IL-10 and TGFβ transcription in endothelial cells, while IL-1β protein levels were elevated in RA FLS albeit to a lower extent than inflammatory mediators. Interestingly, RA FLS remodeled by Syntenin-1 or LPS/IFNγ mutually enhanced GLUT1, HK2, PFK2, and HIF a transcription (Umar S, et al. Cell Mol Life Sci. 2021). GLUT1 and HK2 transcriptional upregulation in LPS/IFNγ reprogrammed RA FLS was suppressed by 2-DG, 10 although HIF1α levels were also negated by IACS, demonstrating its involvement in oxidative stress (Umar S, et al. Cell Mol Life Sci. 2021). Syntenin-1's ability to potentiate mitoATP alongside AMPK in RA FLS contrasted with RA fibroblasts differentiated by LPS/IFNγ or R837, where AMPK levels were unchanged (Umar S, et al. Cell Mol Life Sci. 2021; and Umar S, et al. Life Sci. 2021; 120114). However, distinct from Syntenin-1 or R837 remodeled RA FLS, those reconfigured by LPS/IFNγ displayed citrate accumulation that was resolved by 2-DG and IACS therapy Umar S, et al. Cell Mol Life Sci. 2021; and Umar S, et al. Life Sci. 2021; 120114). The inflammatory profile uncovered in Syntenin-1, R837, and TNF-remodeled RA FLS were commonly abrogated by HIF1αi, while cMYCi disrupted R837 differentiated RA FLS (Umar S, et al. Life Sci. 2021; 120114; and Koedderitzsch K, et al. Sci Rep. 2021; 11:19385). Whereas TLR reprogramming in RA FLS is orchestrated by glycolytic activity, Syntenin-1-driven metabolic profile is dominated by oxidative stress leading to mitochondrial dynamic change through Mitofusin-2 and DRP1.

Interestingly, Syntenin-1, LPS/IFNγ, and IL-6/IFNγ promote RA FLS migration which can be impaired by SDC-1 Ab and mTOR1i (Syntenin-1 activated), glucose uptake blockade (LPS/IFNγ stimulated) or Tofacitinab therapy (IL-6/IFNγ signaling), respectively (Umar S, et al. Cell Mol Life Sci. 2021; and Palasiewicz K, et al. Eur J Immunol. 2021). Contrasting IL-6/IFNγ remodeled RA FLS, those reprogrammed by Syntenin-1 did not display STAT1/3 activation, however, both showed modest glycolytic activity facilitated through HK2 transcription (Palasiewicz K, et al. Eur J Immunol. 2021).

Endothelial cells, RA FLS, or RA explants exposed to Syntenin-1, exhibited an expansion in VEGF/VEGFR and JAG1/Notch1 gene signature. In RA FLS and endothelial cell cocultures, IL-6 was shown to be responsible for VEGF production (Kayakabe K, et al. Rheumatology (Oxford). 2012; 51:1571-1579; and ElshabraNvy H A, et al. Angiogenesis. 2015; 18:433-448). Moreover. IL-6R Ab impaired the synergistic effect of IL-6, IL-1p, and TNF on VEGF production from RA FLS, while the blockade of IL-1β or TNF was ineffective on this manifestation (Elshabrawy H A, et al. Angiogenesis. 2015; 18:433-448; and Nakahara H, et al. Arthritis Rheum. 2003; 48:1521-1529). These findings indicate that escalated IL-6 levels together with TNF and IL-1β detected in Syntenin-1 reprogrammed endothelial cells and RA FLS contribute to the identified proangiogenic gene signature.

In RA explants, HIF1α signaling can widely influence Syntenin-1 mediated inflammatory and pro-angiogenic mediators as well as RAPTOR activity. The inflammatory landscape of Syntenin-1 in endothelial cells and RA ST explants are similarly intercepted by mTORi or HIF1αi. While RA MΦ and Th1 cell reconfiguration by Syntenin-1 are mainly influenced by mTOR activation (Meyer A, et al. Ann Rheum Dis. 2023), RA FLS-mediated inflammation is exclusively modulated by HIF1α signaling in part due to its enriched frequency. Ultimately, the Syntenin-1 arthritic mice portray the involvement of F480⁺Inos^{hi}Arginase^{lo} MΦs (Meyer A, et al. Ann Rheum Dis. 2023), Vimentin⁺ Fibroblasts, and VWF⁺ endothelial cells in advancing joint inflammation, angiogenesis, and hypermetabolic activity that can be counteracted by SDC-1 deficiency. In line with these findings, CIA joint inflammation, vascularization, and immunometabolism were mitigated in SDC-1 KO mice via intercepting, the transcription of IL-6, DLL1/DLL4/JAG2/Notch1, and GLUT1 or mTOR, respectively (Meyer A, et al. Cell Mol Immunol. 2022; 19:1070-1072). In conclusion, the Syntenin-1/SDC1 pathway is integral for RA progression due to its influence on various cell types that manipulate joint inflammation and metabolic malfunction.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A method of treating or preventing rheumatoid arthritis in a subject, the method comprising administering to the subject a therapeutically effective amount of a syndecan-1 inhibitor.

2. A method of reducing syntenin-1 in synovial fluid or blood in a subject, the method comprising administering to the subject a therapeutically effective amount of a syndecan-1 inhibitor.

3. A method of reducing chemokine (C—C motif) ligand 2 (CCL2) levels in a subject, the method comprising administering to the subject a therapeutically effective amount of a syndecan-1 inhibitor.

4. A method of reducing cartilage degradation in a subject, the method comprising administering to the subject a therapeutically effective amount of a syndecan-1 inhibitor.

5. A method of reducing synovial inflammation in a subject, the method comprising administering to the subject, the method comprising administering to the subject a therapeutically effective amount of a syndecan-1 inhibitor.

6. A method of reducing or ameliorating one or more symptoms of rheumatoid arthritis in a subject, the method comprising administering to the subject a therapeutically effective amount of a syndecan-1 inhibitor.

7. The method claim 6, wherein the one or more symptoms of rheumatoid arthritis is pain, joint tenderness, joint swelling, grip strength, or morning stiffness.

8. A method of reducing one or more inflammatory interferon transcription factors in a subject, the method comprising administering to the subject a therapeutically effective amount of a syndecan-1 inhibitor.

9. The method of claim 8, wherein the one or more inflammatory interferon transcription factors are IRF1, IRF7, IRF8, IRF9, or a combination thereof.

10. A method of reducing one or more monokines in subject, the method comprising administering to the subject a therapeutically effective amount of a syndecan-1 inhibitor.

11. The method of claim 10, wherein the one or more monokines are IL-1β, TNF-α, IL-6, IL-8, CCL2, or a combination thereof.

12. A method of reducing expression of one or more glycolytic factors in subject, the method comprising administering to the subject a therapeutically effective amount of a syndecan-1 inhibitor.

13. The method of claim 12, wherein the one or more glycolytic factors are GLUT1, HK2, mTOR, LDHA or a combination thereof.

14. A method of increasing expression of one or more oxidative intermediates or enzymes in subject, the method comprising administering to the subject a therapeutically effective amount of a syndecan-1 inhibitor.

15. The method of claim 14, wherein the oxidative intermediate is AMPK.

16. The method of claim 14, wherein the enzyme is aconitase (ACO2), oxoglutarate dehydrogenase (OGDH), succinate dehydrogenase (SDH2), fumarate hydratase (FH), malate dehydrogenase (MDH), or a combination thereof.

17. The method of any of the preceding claims, wherein the syndecan-1 inhibitor is a monoclonal antibody, a mouse anti-SDCT monoclonal antibody (BA38), BT062-DM4 (indatuximab Ravtansine, VIS832, 4B3, OC-46F2, ULBP2-BB4, CART-138, CD138.CAR, CD138-specific CAR-NK, GLVGLIFAV (SEQ ID NO: 1; PVX-410), or synstatin.

18. The method of any of the preceding claims, wherein the syndecan-1 inhibitor is administered orally, intravenously, or subcutaneously.

19. A method of treating or preventing rheumatoid arthritis in a subject, the method comprising administering to the subject a therapeutically effective amount of a syntenin-1 inhibitor.

20. A method of reducing syntenin-1 in synovial fluid or blood in a subject, the method comprising administering to the subject a therapeutically effective amount of a syntenin-1 inhibitor.

21. A method of reducing chemokine (C—C motif) ligand 2 (CCL2) levels in a subject, the method comprising administering to the subject a therapeutically effective amount of a syntenin-1 inhibitor.

22. A method of reducing cartilage degradation in a subject, the method comprising administering to the subject a therapeutically effective amount of a syntenin-1 inhibitor.

23. A method of reducing synovial inflammation in a subject, the method comprising administering to the subject, the method comprising administering to the subject a therapeutically effective amount of a syntenin-1 inhibitor.

24. A method of reducing or ameliorating one or more symptoms of rheumatoid arthritis in a subject, the method comprising administering to the subject a therapeutically effective amount of a syntenin-1 inhibitor.

25. The method claim 24, wherein the one or more symptoms of rheumatoid arthritis is pain, joint tenderness, joint swelling, grip strength, or morning stiffness.

26. A method of reducing one or more inflammatory interferon transcription factors in subject, the method comprising administering to the subject a therapeutically effective amount of a syntenin-1 inhibitor.

27. The method of claim 26, wherein the one or more inflammatory interferon transcription factors are IRF1, IRF7, IRF8, IRF9, or a combination thereof.

28. A method of reducing one or more monokines in subject, the method comprising administering to the subject a therapeutically effective amount of a syntenin-1 inhibitor.

29. The method of claim 28, wherein the one or more monokines are IL-1β, TNF-α, IL-6, IL-8, CCL2, or a combination thereof.

30. A method of reducing expression of one or more of glycolytic factors in subject, the method comprising administering to the subject a therapeutically effective amount of a syntenin-1 inhibitor.

31. The method of claim 30, wherein the one or more glycolytic factors are GLUT1, HK2, mTOR, LDHA or a combination thereof.

32. A method of increasing expression of one or more of oxidative intermediates or enzymes in subject, the method comprising administering to the subject a therapeutically effective amount of a syntenin-1 inhibitor.

33. The method of claim 32, wherein the oxidative intermediate is AMPK.

34. The method of claim 32, wherein the enzyme is aconitase (ACO2), oxoglutarate dehydrogenase (OGDH), succinate dehydrogenase (SDH2), fumarate hydratase (FH), malate dehydrogenase (MDH), or a combination thereof.

35. A method of treating or preventing juvenile idiopathic arthritis in a subject, the method comprising administering to the subject a therapeutically effective amount of a syndecan-1 inhibitor.

36. A method of treating or preventing psoriatic arthritis in a subject, the method comprising administering to the subject a therapeutically effective amount of a syndecan-1 inhibitor.

37. A method of treating or preventing ankylosing spondylitis in a subject, the method comprising administering to the subject a therapeutically effective amount of a syndecan-1 inhibitor.

38. A method of treating or preventing Crohn's disease in a subject, the method comprising administering to the subject a therapeutically effective amount of a syndecan-1 inhibitor.

39. A method treating or preventing ulcerative colitis in a subject, the method comprising administering to the subject a therapeutically effective amount of a syndecan-1 inhibitor.

40. A method treating or preventing plaque psoriasis in a subject, the method comprising administering to the subject a therapeutically effective amount of a syndecan-1 inhibitor.

41. A method treating or preventing hidradenitis suppurativa in a subject, the method comprising administering to the subject a therapeutically effective amount of a syndecan-1 inhibitor.

42. A method treating or preventing uveitis in a subject, the method comprising administering to the subject a therapeutically effective amount of a syndecan-1 inhibitor.

43. A method of reducing or ameliorating one or more symptoms of idiopathic juvenile arthritis in a subject, the method comprising administering to the subject a therapeutically effective amount of a syndecan-1 inhibitor.

44. The method claim 43, wherein the one or more symptoms of rheumatoid arthritis is pain, joint tenderness, joint swelling, grip strength, morning stiffness, eye inflammation, fatigue, decreased appetite, poor weight gain, slow growth, high fever, rash, or swollen lymph nodes.

45. A method of reducing or ameliorating one or more symptoms of psoriatic arthritis in a subject, the method comprising administering to the subject a therapeutically effective amount of a syndecan-1 inhibitor.

46. The method of claim 45, wherein the one or more symptoms psoriatic arthritis is pain, joint tenderness, joint swelling morning stiffness, itching, tendinopathy, skin or rash.

47. A method of reducing or ameliorating one or more symptoms of ankylosing spondylitis in subject, the method comprising administering to the subject a therapeutically effective amount of a syndecan-1 inhibitor.

48. The method of claim 47, wherein the one or more symptoms ankylosing spondylitis is pain, joint tenderness, morning stiffness, stooped posture, appetite loss, weight loss, fatigue, fever, anemia, eye inflammation, blurred vision or sensitivity to light, back joint dysfunction or inflammatory bowel disease.

49. The method of any of the preceding claims, wherein the syndecan-1 inhibitor is a monoclonal antibody, a mouse anti-SDC1 monoclonal antibody (BA38), BT062-DM4 (indatuximab Ravtansine, VIS832, 4B3, OC-46F2, ULBP2-BB4, CART-138, CD138.CAR, CD138-specific CAR-NK, GLVGLIFAV (SEQ ID NO: 1; PVX-410), or synstatin.

50. The method of any of the preceding claims, wherein the syndecan-1 inhibitor is administered orally, intravenously, or subcutaneously.

51. A method of treating or preventing rheumatoid arthritis in a subject, the method comprising administering to the subject a therapeutically effective amount of a syntenin-1 inhibitor.

52. A method of reducing syntenin-1 in synovial fluid or blood in a subject, the method comprising administering to the subject a therapeutically effective amount of a syntenin-1 inhibitor.

53. A method of reducing chemokine (C—C motif) ligand 2 (CCL2) levels in a subject, the method comprising administering to the subject a therapeutically effective amount of a syntenin-1 inhibitor.

54. A method of reducing cartilage degradation in a subject, the method comprising administering to the subject a therapeutically effective amount of a syntenin-1 inhibitor.

55. A method of reducing synovial inflammation in a subject, the method comprising administering to the subject a therapeutically effective amount of a syntenin-1 inhibitor.

56. A method of reducing or ameliorating one or more symptoms of rheumatoid arthritis in a subject, the method comprising administering to the subject a therapeutically effective amount of a syntenin-1 inhibitor.

57. The method claim 56, wherein the one or more symptoms of rheumatoid arthritis is pain, joint tenderness, joint swelling, grip strength, or morning stiffness.

58. A method of reducing one or more inflammatory interferon transcription factors in subject, the method comprising administering to the subject a therapeutically effective amount of a syntenin-1 inhibitor.

59. The method of claim 58, wherein the one or more inflammatory interferon transcription factors are IRF1, IRF7, IRF8, IRF9, or a combination thereof.

60. A method of reducing one or more monokines in subject, the method comprising administering to the subject a therapeutically effective amount of a syntenin-1 inhibitor.

61. The method of claim 60, wherein the one or more monokines are IL-1β, TNF-α, IL-6, IL-8, CCL2, or a combination thereof.

62. A method of reducing expression of one or more of glycolytic factors in subject, the method comprising administering to the subject a therapeutically effective amount of a syntenin-1 inhibitor.

63. The method of claim 62, wherein the one or more glycolytic factors are GLUT1, HK2, mTOR, LDHA or a combination thereof.

64. A method of increasing expression of one or more of oxidative intermediates or enzymes in subject, the method comprising administering to the subject a therapeutically effective amount of a syntenin-1 inhibitor.

65. The method of claim 64, wherein the oxidative intermediate is AMPK.

66. The method of claim 64, wherein the enzyme is aconitase (ACO2), oxoglutarate dehydrogenase (OGDH), succinate dehydrogenase (SDH2), fumarate hydratase (FH), malate dehydrogenase (MDH), or a combination thereof.

67. A method of treating or preventing juvenile idiopathic arthritis in a subject, the method comprising administering to the subject a therapeutically effective amount of a syntenin-1 inhibitor.

68. A method of treating or preventing psoriatic arthritis in a subject, the method comprising administering to the subject a therapeutically effective amount of a syntenin-1 inhibitor.

69. A method of treating or preventing ankylosing spondylitis in a subject, the method comprising administering to the subject a therapeutically effective amount of a syntenin-1 inhibitor.

70. A method of treating or preventing Crohn's disease in a subject, the method comprising administering to the subject a therapeutically effective amount of a syntenin-1 inhibitor.

71. A method treating or preventing ulcerative colitis in a subject, the method comprising administering to the subject a therapeutically effective amount of a syntenin-1 inhibitor.

72. A method treating or preventing plaque psoriasis in a subject, the method comprising administering to the subject a therapeutically effective amount of a syntenin-1 inhibitor.

73. A method treating or preventing hidradenitis suppurativa in a subject, the method comprising administering to the subject a therapeutically effective amount of a syntenin-1 inhibitor.

74. A method treating or preventing uveitis in a subject, the method comprising administering to the subject a therapeutically effective amount of a syntenin-1 inhibitor.

75. A method of reducing or ameliorating one or more symptoms of idiopathic juvenile arthritis in a subject, the method comprising administering to the subject a therapeutically effective amount of a syntenin-1 inhibitor.

76. The method claim 75, wherein the one or more symptoms of idiopathic juvenile arthritis is pain, joint tenderness, joint swelling, grip strength, morning stiffness, eye inflammation, fatigue, decreased appetite, poor weight gain, slow growth, high fever, rash, or swollen lymph nodes.

77. A method of reducing or ameliorating one or more symptoms of psoriatic arthritis in a subject, the method comprising administering to the subject a therapeutically effective amount of a syntenin-1 inhibitor.

78. The method of claim 77, wherein the one or more symptoms psoriatic arthritis is pain, joint tenderness, joint swelling morning stiffness, itching, tendinopathy, skin or rash.

79. A method of reducing or ameliorating one or more symptoms of ankylosing spondylitis in subject, the method comprising administering to the subject a therapeutically effective amount of a syntenin-1 inhibitor.

80. The method of claim 80, wherein the one or more symptoms ankylosing spondylitis is pain, joint tenderness, morning stiffness, stooped posture, appetite loss, weight loss, fatigue, fever, anemia, eye inflammation, blurred vision or sensitivity to light, back joint dysfunction or inflammatory bowel disease.

81. The method of any of the preceding claims, wherein the subject is a human patient.

82. The method of any of the preceding claims, wherein the subject has rheumatoid arthritis or is obese.

83. The method of any of the preceding claims, wherein the syntenin-1 inhibitor or syndecan-1 inhibitor prevents syndecan-1 from binding to the PDZ-2 domain of syntenin-1.

84. The method of any of the preceding claims, wherein the syntenin-1 inhibitor binds to the PDZ-2 domain of syntenin-1.

85. The method of claim 84, wherein the syntenin-1 inhibitor binds to the PDZ-2 domain of syntenin-1 thereby preventing syndecan-1 from binding to the PDZ-2 domain of syntenin-1.

86. The method of any of the preceding claims, further comprising administering at least a second therapeutic agent to the subject.

87. The method of claim 86, wherein the second therapeutic agent is a nonsteroidal anti-inflammatory drug, a disease modifying anti-rheumatic drug or a joint replacement surgery.

88. The method of any of the preceding claims, wherein the syntenin-1 inhibitor is administered orally, intravenously, or subcutaneously.

89. The method of any of the preceding claims, wherein T effector cell differentiation into the Th1 or Th17 subtype is modulated/prevented in the subject.


SEQUENCE LISTING (1 sequence)

SEQ ID NO: 1 - protein, 9 aa, synthetic construct
GLVGLIFAV